SEQUENCE LISTING (18 sequences)

SEQ ID NO: 1 - protein, 1368 aa, synthetic construct [sequence omitted]

SEQ ID NO: 2 - DNA, 220 bp, synthetic construct
  misc_feature 1..220: note = MDH promoter MP2
  source 1..220: mol_type = other DNA
tttgcctcgatcggcggtccttgtgacagggagatattcccgacggatccggggcattcgagcggaaccgcccgccgtgggagtttttccagcgagcattcgagagtttttcaaggcggcttcgaggggttattccgtaacgccgccgacatgatctgtcccagaatctccgccgctgttcgtagagcgccgatgcagggtcggcatcaatcattcttgg

SEQ ID NO: 3 - DNA, 271 bp, synthetic construct
  misc_feature 1..271: note = LacI inducible MDH promoter MP2
  source 1..271: mol_type = other DNA
aattgtgagcggataacaatttttgcctcgatcggcggtccttgtgacagggagatattcccgacggatccggggcattcgagcggaaccgcccgccgtgggagtttttccagcgagcattcgagagtttttcaaggcggcttcgaggggttattccgtaacgccgccgacatgatctgtcccagaatctccgccgctgttcgtagagcgccgatgcagggtcggcatcaatcattcttggtgtggaattgtgagcgctcacaattccaca

SEQ ID NO: 4 - DNA, 107 bp, synthetic construct
  misc_feature 1..107: note = Sodium benzoate inducible promoter
  source 1..107: mol_type = other DNA
gcgacgtgcgcctggcggatagcgatgtgcaggcagcggatattgacgggcagggcgagcacgtacggtgagggcgcctgatacaagaacaacggagggcccgcccc

SEQ ID NO: 5 - DNA, 50 bp, synthetic construct
  misc_feature 1..50: note = Synthetic pBba promoter
  source 1..50: mol_type = other DNA
gaattctaaagatctttgacagctagctcagtcctaggtataatactagt

SEQ ID NO: 6 - DNA, 696 bp, synthetic construct
  misc_feature 1..696: note = moxF promoter MP12
  source 1..696: mol_type = other DNA
gcgccgtatgctttcgaatccgccaagaccgggcatggataaatccatgaccaccacatcgggcaattgctttgaatagagttggcaagctgtctcgccacgatcggcttcatagatctcgccgatacgatccgacagcgatagataggtcttgtagccggtacgaaccacggcgtgatcatctaccaataaaacgctgattttactcgccactggaaaatttcctcctcaggtcgtcaagggataaagatatgggacaagtccagtctgatgccaggcggacttggtgtgcctttttttatgatgacgctttatccgtgcttaaaccatgggagcttttcccgtttccaatttcgatccttggcgagataggaatatttccgtgcatgattgcgtcgatttcacatcgatttcatggattgttccgtaacgttagccagcccggcttctataacatttgcgccagcgtggcctggtggtcggtaacccgtgatgcggttatgatcaacaaagctggttttcaacgactaattctgatcttcaggtcgcgcctcacttatagcgataaaaatcctggaggaaacatgcaacaactcgatttgcgcatagtcgggaaaaccgcggccttgttggctggtggccttctgagcgtggcgcaacccgcatcggcgaacaaggagctggaacagctctcca

SEQ ID NO: 7 - DNA, 65 bp, synthetic construct
  misc_feature 1..65: note = transcriptional terminator
  source 1..65: mol_type = other DNA
tttttttgaagcttgggcccgaacaaaaactcatctcagaagaggatctgaatagcgccgtcgac

SEQ ID NO: 8 - DNA, 43 bp, synthetic construct
  misc_feature 1..43: note = Hammerhead self-cleaving ribozyme
  source 1..43: mol_type = other DNA
cgactactgatgagtccgtgaggacgaaacgagtaagctcgtc

SEQ ID NO: 9 - DNA, 72 bp, synthetic construct
  misc_feature 1..72: note = HDV rself-cleaving ribozyme
  source 1..72: mol_type = other DNA
ttttggccggcatggtcccagcctcctcgctggcgccggctgggcaacatgcttcggcatggcgaatgggac

SEQ ID NO: 10 - protein, 138 aa, synthetic construct
  REGION 1..138: note = Gam recombinase
MDINTETEIKQKHSLTPFPVFLISPAFRGRYFHSYFRSSAMNAYYIQDRLEAQSWARHYQQLAREEKEAELADDMEKGLPQHLFESLCIDHLQRHGASKKSITRAFDDDVEFQERMAEHIRYMVETIAHHQVDIDSEV

SEQ ID NO: 11 - protein, 261 aa, synthetic construct
  REGION 1..261: note = Bet recombinase
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 12 - protein, 226 aa, synthetic construct
  REGION 1..226: note = Exo recombinase
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 13 - DNA, 80 bp, synthetic construct
  misc_feature 1..80: note = E.coli_rrnBT1 terminator
  source 1..80: mol_type = other DNA
ccaggcatcaaataaaacgaaaggctcagtcgaaagactgggcctttcgttttatctgttgtttgtcggtgaacgctctc

SEQ ID NO: 14 - DNA, 129 bp, synthetic construct
  misc_feature 1..129: note = Terminator E. coli_rrnBT1 and terminator T7 combined
  source 1..129: mol_type = other DNA
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 15 - DNA, 48 bp, synthetic construct
  misc_feature 1..48: note = Terminator Bath_pmoABC1_Fd
  source 1..48: mol_type = other DNA
caacgccaaaacccccggccggcaacggtcgggggttttttattctgg

SEQ ID NO: 16 - DNA, 47 bp, synthetic construct
  misc_feature 1..47: note = Terminator Bath_pmoABC2_Fd
  source 1..47: mol_type = other DNA
caacgccaaaacccccggccggcaacggtcgggggtttttttatcgg

SEQ ID NO: 17 - DNA, 54 bp, synthetic construct
  misc_feature 1..54: note = Terminator Bath_rrfA
  source 1..54: mol_type = other DNA
tcatcccaatcaaccccgagtccctccccaggaactcggggtttttatttaggg

SEQ ID NO: 18 - DNA, 75 bp, synthetic construct
  misc_feature 1..75: note = specR targeting oligonucleotide
  source 1..75: mol_type = other DNA
agagcgaaatcctccgcgctgtagaagtcaccattgtttagcacgacgacatcattccgtggcgttatccagcta

(19) United States
(12) Patent Application Publication
(10) Pub. No.: US 2024/0043877 A1
(43) Pub. Date: Feb. 8, 2024

(54) COMPOSITIONS AND METHODS FOR GENETIC MANIPULATION OF METHANOTROPHS

(71) Applicant: Calysta, Inc., San Mateo, CA (US)

(72) Inventors: Jeremy Minshull, Los Altos, CA (US); Jana Stumpe, Sunnyvale, CA (US); Sonny Zhang, Pleasanton, CA (US); Megan Hsi, San Mateo, CA (US)

(21) Appl. No.: 18/491,636

(22) Filed: Oct. 20, 2023

Related U.S. Application Data
(63) Continuation of application No. 17/095,644, filed on Nov. 11, 2020, now abandoned.
(60) Provisional application No. 62/939,468, filed on Nov. 22, 2019.

(51) Int. Cl. C12N 15/90; C12N 15/11; C12N 9/22

(52) U.S. Cl. CPC C12N 15/902; C12N 15/11; C12N 9/22; C12N 2800/80; C12N 2310/20

(57) ABSTRACT

The present disclosure provides compositions and methods for the genetic manipulation of methanotrophs utilizing a site-specific polynucleotide modification system.

### REFERENCE TO AN ELECTRONIC SEQUENCE LISTING

The contents of the electronic sequence listing (421C1_SeqListing.xml; Size: 27,479 bytes; and Date of Creation: Oct. 20, 2023) is herein incorporated by reference in its entirety.

### BACKGROUND

Methanotrophic bacteria require single-carbon compounds to survive and are able to metabolize methane as their only source of carbon and energy. They are of special interest in reducing the release of methane into the atmosphere from high methane-producing environments and in reducing certain environmental contaminants such as chlorinated hydrocarbons.

In spite of the importance of methanotrophic bacteria, genetic manipulation of such bacteria has historically been difficult due to a lack of robust protocols and tools such as vectors, expression cassettes, and suitable promoters (see, e.g., Ali and Murrel, Microbiology 155:761-71, 2009). Where such tools exist, various issues have hindered their use, including methanotroph-incompatible antibiotic resistance markers, inappropriate restriction sites, and poorly expressed proteins. Creating chromosomal modifications in methanotrophic bacteria has been limited to homologous recombination using counter-selectable markers, such as sacB. For example, engineering the Methylococcus capsulatus Bath genome by homologous recombination is a time consuming and experimentally cumbersome process that generally takes 4-6 weeks. An additional disadvantage of this method is that genome modifications can only be introduced sequentially.

Recently the Clustered Regularly Interspaced Short Palindromic Repeats (CRISPR) Type II system, derived from Streptococcus pyogenes, has emerged as a promising RNA-guided endonuclease technology for genome engineering in eukaryotes. The CRISPR/Cas system was first discovered in bacteria and protects bacteria and archaea against phages and plasmids in a sequence-specific manner (see, e.g., Xu et al., Appl Environ Microbiol., 80:1544-52, 2014). The native CRISPR/Cas system integrates short repeats of phage or plasmid DNA into the bacterial genome, and upon reinfection, transcripts of these repeats guide a nuclease (e.g., Cas9) to the invading complementary DNA and destroy it. CRISPR/Cas systems have been used successfully in eukaryotic model organisms and E. coli, which have well established tools for genetic manipulation, to introduce targeted DNA cleavage.

Though some progress has been made in the development of molecular biology tools for engineering methanotrophs, more are needed to develop engineered methanotrophs suitable for producing commercially desired products.

### SUMMARY

In one aspect, the present disclosure provides methods of altering the genome of a methanotrophic bacterium, comprising culturing under conditions and for a time sufficient to allow expression in a methanotrophic bacterium of a site-specific polynucleotide modification system; wherein the methanotrophic bacterium contains a heterologous nucleic acid molecule encoding the site-specific polynucleotide modification system that is operably linked to a regulatory element in a vector, the nucleic acid molecule comprising: (a) a first heterologous nucleic acid molecule encoding a modification polypeptide, wherein the modification polypeptide comprises a targeting RNA binding domain and a site-specific nuclease domain, and (b) a second heterologous nucleic acid molecule encoding a targeting RNA, wherein the targeting RNA comprises a duplex-forming region and a DNA-targeting domain, wherein the complex of the expressed modification polypeptide with the expressed targeting RNA binds to and cleaves a genomic target sequence of the methanotrophic bacterium, thereby site-specifically altering the genome of the methanotrophic bacterium.

In another aspect, the present disclosure provides modified methanotrophs, comprising a heterologous nucleic acid molecule encoding a site specific polynucleotide modification system that is operably linked to a regulatory element in a vector, the nucleic acid molecule comprising: (a) a first heterologous nucleic acid molecule encoding a modification polypeptide, wherein the modification polypeptide comprises a targeting RNA binding domain and a site-specific nuclease domain, (b) a second heterologous nucleic acid molecule encoding a targeting RNA, wherein the targeting RNA comprises a duplex forming region and a DNA-targeting domain, and (c) a third heterologous nucleic acid molecule comprising an integration polynucleotide, wherein the expressed modification polypeptide can associate with the expressed targeting RNA to form a complex capable of binding to and cleaving a genomic target sequence of the methanotroph.

### BRIEF DESCRIPTION OF THE DRAWINGS

FIG. 1 is a schematic representation of an exemplary expression vector “Plasmid 1” comprising sequences encoding a replication initiation protein (trfA), an origin of vegetative replication (oriV) that is functional in methanotrophic bacteria, an origin of transfer (oriT), an origin of replication for E. coli (pUC ori), a lad repressor protein (lacI) operably linked to a constitutive promoter that is functional in methanotrophs (e.g., 30S ribosomal protein S16 promoter MP10), and a kanamycin resistance gene (KanR). Plasmid 1 also comprises an expression cassette comprising sequences encoding a modification polypeptide (e.g., Cas9) and a recombinase (e.g., Gam, Bet, Exo), which are operably linked to a Lad inducible methanol dehydrogenase (MDH) promoter (MP2, SEQ ID NO:3). Plasmid 1 can be used to carry expression cassettes comprising modification polypeptides of the present disclosure for site-specific genetic engineering or modification of methanotrophic bacteria.

FIG. 2 is a schematic representation of an exemplary pUC-based plasmid “Plasmid 2.1” comprising an integration polynucleotide cassette comprising sequences encoding a donor molecule (e.g., spectinomycin resistance gene) flanked by a PAM sequence, MCA0775 target sequence that is complementary to the DNA-targeting domain of the targeting RNA encoded on the same plasmid, and 5′ homology flank segment on one end, and a PAM sequence, MCA0775 target sequence that is complementary to the DNA-targeting domain of the targeting RNA encoded on the same plasmid, and 3′ homology flank segment on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 2.1 also comprises a targeting RNA (e.g., sgRNA), which is flanked on the 5′-end and 3′-end by a self-cleaving ribozyme; which are operably linked to a constitutive promoter that is functional in methanotrophs (e.g., moxF promoter, MP12, SEQ ID NO:6). The ribozyme at the 5′-end cleaves at its 3′-end, and the ribozyme at the 3′-end cleaves at its 5′-end. Thus, the cleavage by the two ribozymes allows the targeting RNA to be released. Plasmid 2.1 also comprises a counter selectable marker (e.g., sacB), an origin of transfer (oriT), an origin of replication for E. coli (pUC ori), and a kanamycin resistance gene (KanR). Plasmid 2.1 is a pUC-based plasmid and unable to replicate in methanotrophic bacteria. Plasmid 2.1 can be used to carry expression cassettes comprising targeting RNAs of the present disclosure and a donor molecule for integration into the cleaved methanotroph target DNA.

FIG. 3 is a schematic representation of an exemplary pUC-based plasmid “Plasmid 2.2” comprising a integration polynucleotide cassette comprising sequences encoding a donor molecule (e.g., spectinomycin resistance gene) flanked by a PAM sequence, MCA0775 target sequence, and 5′ homology flank segment on one end and a PAM sequence, MCA0775 target sequence and 3′ homology flank segment on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 2.2 also comprises a targeting RNA (e.g., sgRNA), which is operably linked to a constitutive promoter that is functional in methanotrophs (e.g., synthetic promoter pBba, SEQ ID NO:5) and a transcriptional terminator. Plasmid 2.2 also comprises a counter selectable marker (e.g., sacB), an origin of transfer (oriT), an origin of replication for E. coli (pUC ori), and a kanamycin resistance gene (KanR). Plasmid 2.2 is a pUC-based plasmid and unable to replicate in methanotrophic bacteria. Plasmid 2.2 can be used to carry expression cassettes comprising targeting RNAs of the present disclosure and a donor molecule for integration into the cleaved methanotroph target DNA.

FIG. 4 is a schematic representation of an exemplary pUC-based plasmid “Plasmid 2.3” comprising an integration polynucleotide cassette comprising sequences encoding a donor molecule (e.g., spectinomycin resistance gene) flanked by a PAM sequence, MCA0775 target sequence, and 5′ homology flank segment on one end and a PAM sequence, MCA0775 target sequence, and 3′ homology flank segment on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 2.3 also comprises a targeting RNA (e.g., sgRNA), which is operably linked to a constitutive promoter that is functional in methanotrophs (e.g., moxF promoter MP12, SEQ ID NO:6) and a transcriptional terminator. Plasmid 2.3 also comprises a counter selectable marker (e.g., sacB), an origin of transfer (oriT), an origin of replication for E. coli (pUC ori), and a kanamycin resistance gene (KanR). Plasmid 2.3 is a pUC-based plasmid and unable to replicate in methanotrophic bacteria. Plasmid 2.3 can be used to carry expression cassettes comprising targeting RNAs of the present disclosure and a donor molecule for integration into the cleaved methanotroph target DNA.

FIG. 5 is a schematic representation of an exemplary pUC-based plasmid “Plasmid 3” comprising an integration polynucleotide cassette comprising sequences encoding spectinomycin resistance marker, a constitutive promoter that is functional in methanotrophs (e.g., 30S ribosomal protein S16 promoter MP10), a lad repressor protein (lacI), a Lad inducible methanol dehydrogenase (MDH) promoter (MP2, SEQ ID NO:3), DNA insert, which are flanked by a PAM sequence, MCA0775 target sequence, and 5′ homology flank segment on one end and a PAM sequence, MCA0775 target sequence, and 3′ homology flank segment on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 3 also comprises sequences encoding a targeting RNA (e.g., sgRNA), which is operably linked to a constitutive promoter that is functional in methanotrophs (e.g., pBba promoter, SEQ ID NO:5) and a transcriptional terminator, a counter selectable marker (e.g., sacB), an origin of transfer (oriT), an origin of replication for E. coli (pUC ori), and a kanamycin resistance gene (KanR). Plasmid 3 is a pUC-based plasmid and unable to replicate in methanotrophic bacteria. Plasmid 3 can be used to carry expression cassettes comprising targeting RNAs of the present disclosure and a donor molecule for integration into the cleaved methanotroph target DNA.

FIG. 6 is a schematic representation of an exemplary pUC-based plasmid “Plasmid 4” comprising an integration polynucleotide cassette comprising sequences encoding spectinomycin resistance marker, a constitutive promoter that is functional in methanotrophs (e.g., 30S ribosomal protein S16 promoter MP10), a lad repressor protein (lacI), a Lad inducible methanol dehydrogenase (MDH) promoter (MP2, SEQ ID NO:3), and a donor molecule (e.g., metabolic pathway enzyme), which are flanked by a PAM sequence, MCA0775 target sequence, and 5′ homology flank segment on one end and a PAM sequence, MCA0775 target sequence, and 3′ homology flank segment on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 4 also comprises sequences encoding a targeting RNA (e.g., sgRNA), which is operably linked to a constitutive promoter that is functional in methanotrophs (e.g., synthetic pBba promoter, SEQ ID NO:5) and a transcriptional terminator, a counter selectable marker (e.g., sacB), an origin of transfer (oriT), an origin of replication for E. coli (pUC ori), and a kanamycin resistance gene (KanR). Plasmid 4 is a pUC-based plasmid and unable to replicate in methanotrophic bacteria. Plasmid 4 can be used to carry expression cassettes comprising targeting RNAs of the present disclosure and a donor molecule for integration into the cleaved methanotroph target DNA.

FIG. 7 is a schematic representation of an exemplary pUC-based plasmid “Plasmid 5.1” comprising sequences encoding an origin of vegetative replication (oriV) that is functional in methanotrophic bacteria, an origin of transfer (oriT), an origin of replication for E. coli (pUC ori), and a spectinomycin resistance marker. Plasmid 5 also comprises a sequence encoding a replication initiation protein (trfA), and a sequence encoding a targeting RNA (e.g., sgRNA), which is operably linked to a constitutive promoter functional in methanotrophs (e.g., pBba, SEQ ID NO:5) and a transcriptional terminator (e.g., rrnB_txn_terminator, SEQ ID NO:13).

FIG. 8 depicts growth of M. capsulatus Bath G680 cells transformed with MCA0755-targeting Plasmid 5.1, Plasmid 5.2, or Plasmid 5.3 (from left to right) on MM-W1 agar plates. The bottom row shows significant colony growth for M. capsulatus Bath G680 cells that have been transformed with MCA0755-targeting plasmid 5.1, 5.2, or 5.3, but lacking a plasmid conferring Cas9 activity on spectinomycin containing MM-W1 agar plates. The top row shows very little colony growth of M. capsulatus Bath G680 cells transformed with each MCA0755 targeting plasmid and the Cas9-containing Plasmid 1, suggesting that RNA guided cleavage of MCA0755 by Cas9 was sufficient to kill the host M. capsulatus Bath G680 cells.

FIG. 9 is a schematic representation of an exemplary pUC-based gene disruption plasmid, MCA1474-targeting plasmid “Plasmid 6.1”, comprising an integration polynucleotide cassette comprising sequences encoding spectinomycin resistance gene and SacB resistance marker which are flanked by a PAM sequence, MCA1474 target sequence and 5′ homology flank segment on one end, and a PAM sequence, MCA1474 target sequence, 3′ homology flank segment, and repeat region that is homologous to a repeat region adjacent to or in the vicinity of the methanotroph host cell genomic target site, on the other end. The 5′ and 3′ homology flank segments are homologous to the 5′ upstream and 3′ downstream sequences of the methanotroph target DNA cleavage site within the MCA1474 locus generated by the MCA1474-specific polynucleotide modification system. Inclusion of PAM sequence and portion of target sequence, which are complementary to the DNA targeting domain of the MCA1474 targeting RNA, flanking the donor resistance gene and marker gene facilitates their integration into the host methanotroph genome at the target DNA cleavage site. Plasmid 6.1 also comprises sequences encoding a MCA1474-targeting RNA (e.g., MCA1474 specific DNA targeting domain and a duplex forming region (aka. scaffold RNA)), which is operably linked to a constitutive promoter that is functional in methanotrophs (e.g., pBba promoter, SEQ ID NO:5) and a transcriptional terminator (e.g., rrnB_txn_terminator, SEQ ID NO:13), an origin of transfer (oriT), and an origin of replication for E. coli (pUC ori).

FIG. 10 is a schematic representation of an exemplary pUC-based gene disruption plasmid, 1*H-targeting plasmid “Plasmid 7.1”, comprising an integration polynucleotide cassette comprising sequences encoding a spectinomycin resistance gene and SacB resistance marker, which are flanked by a PAM sequence, MCA0229 target sequence, 5′ homology flank segment, and repeat region that is homologous to a repeat region adjacent to or in the vicinity of the methanotroph host cell genomic target site, on one end, and a PAM sequence, MCA0229 target sequence, and 3′ homology flank segment on the other end. The 5′ and 3′ homology flank segments are homologous to the 5′ upstream and 3′ downstream sequences of the methanotroph target DNA cleavage site within the MCA0229 locus generated by the MCA0229-specific polynucleotide modification system. Inclusion of PAM sequence and portion of target sequence, which are complementary to the DNA targeting domain of the MCA0229-targeting RNA, flanking the donor resistance gene and marker gene facilitates their integration into the host methanotroph genome at the target DNA cleavage site. Introduction of a homologous repeat region by the integration polynucleotide cassette at the methanotroph target site allows for convenient subsequent loop out of the spectinomycin and SacB genes. Plasmid 7.1 also comprises sequences encoding a MCA0229-targeting RNA (e.g., MCA0229 specific DNA targeting domain and a duplex forming region (aka. scaffold RNA)), which is operably linked to a constitutive promoter that is functional in methanotrophs (e.g., pBba, SEQ ID NO:5) and a transcriptional terminator (e.g., rrnB_txn_terminator, SEQ ID NO:13), an origin of transfer (oriT), an origin of replication for E. coli (pUC ori).

FIG. 11 is a schematic representation of an exemplary pUC-based gene disruption plasmid, MCA0775-targeting “Plasmid 8.1”, comprising an integration polynucleotide cassette comprising sequences encoding spectinomycin resistance, which are flanked by a PAM sequence, MCA0775 target sequence, and 5′ homology flank segment on one end and a PAM sequence, MCA0775 target sequence and 3′ homology flank segment, on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 8.1 also comprises sequences encoding a MCA0775-targeting RNA (e.g., MCA0775 specific DNA targeting domain and a duplex forming region (aka. scaffold RNA)), which is operably linked to a constitutive promoter that is functional in methanotrophs (e.g., pBba, SEQ ID NO:5) and a transcriptional terminator (e.g., rrnB_txn_terminator, SEQ ID NO:13), an origin of transfer (oriT), an origin of replication for E. coli (pUC ori), a kanamycin resistance gene, and counterselectable marker SacB gene.

FIGS. 12A-12D is a schematic representation of an exemplary target site or “WT locus” (FIG. 12A), an exemplary pUC-based gene disruption plasmid comprising an expression cassette comprising a targeting RNA of the present disclosure and an integration polynucleotide cassette comprising a donor molecule(s) (e.g., spectinomycin resistance gene and sacB marker gene) for integration into the cleaved methanotroph target site (FIG. 12B), the deletion locus on the methanotroph genome following targeted gene disruption and integration of the donor molecule (FIG. 12C), and the looped out locus at the methanotroph target site (FIG. 12D). Introduction of a homologous repeat region by the integration polynucleotide cassette at the methanotroph target site allows for convenient subsequent loop out of the spectinomycin and SacB markers, thus allowing for a markerless deletion integration/deletion system.

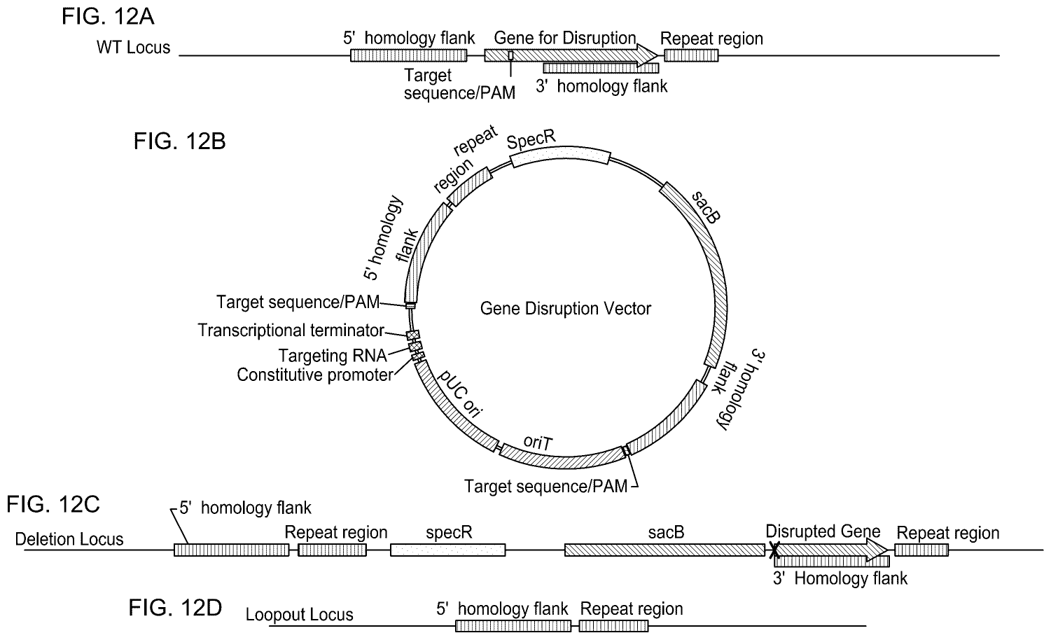

FIG. 13 depicts growth of M. capsulatus Bath wild-type cells transformed with MCA0775-targeting Plasmid 8.1, MCA1474-targeting Plasmid 6.1, or MCA0229-targeting Plasmid 7.1, and Cas9-containing Plasmid 1 (top row) and lacking Cas9-containing Plasmid 1 (bottom row). Greater than 10-fold increase in conjugation frequency was observed in host M. capsulatus Bath wild-type cells transformed with a given gene disruption plasmid and the Cas9-containing Plasmid 1 as compared to without the Cas9-containing Plasmid 1, indicating that the presence of the Cas-9 containing plasmid is necessary to improve the integration rates of the gene disruption plasmids.

FIG. 14 depicts PCR screening for spectinomycin and SacB integration at the targeted locus in M. capsulatus Bath G680 cells transformed with Cas9-containing Plasmid 1 and the glgC- or nifH-targeting plasmid. Eight colonies of each targeting plasmid transformation were screened, four of which are shown compared with wild type M. capsulatus Bath. All eight colonies for each targeting plasmid showed a disrupted target locus with the expected deletion locus amplicon size and expected looped out segment from the targeted M. capsulatus Bath G680 genome locus.

FIG. 15 depicts PCR screening for spectinomycin and SacB integration at the targeted locus M. capsulatus Bath G680 cells transformed with Cas9-containing Plasmid 1 and the MCA0775-targeting plasmid. The eight colonies that were screened showed a disrupted target locus with the expected deletion locus amplicon size.

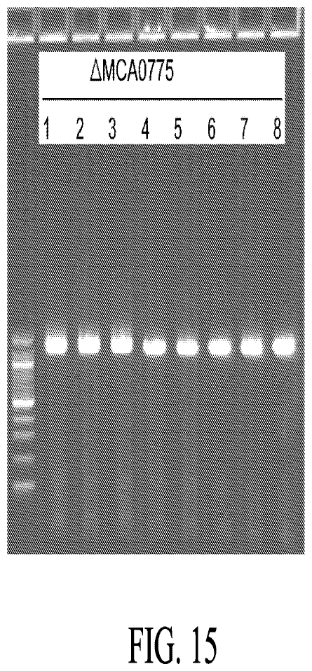

FIG. 16 is a schematic representation of an exemplary pUC-based gene disruption plasmid, MCA0775-targeting “Plasmid 9”, comprising an integration polynucleotide cassette comprising sequences encoding gentamicin resistance gene (gentR) and an inactivated version of spectinomycin resistance gene with point mutations (G274T, T275A) (specR inactive), which are flanked by a PAM sequence, MCA0775 target sequence, and 5′ homology flank segment on one end and a PAM sequence, MCA0775 target sequence and 3′ homology flank segment, on the other end. The 5′ and 3′ homology flank segments are homologous to the 5′ upstream and 3′ downstream sequences of the methanotroph target DNA cleavage site within the MCA0775 locus generated by the MCA0775-specific polynucleotide modification system. Plasmid 9 also comprises sequences encoding a MCA0775-targeting RNA (e.g., MCA0775 specific DNA targeting domain and a duplex forming region (aka. scaffold RNA)), which is operably linked to a constitutive promoter that is functional in methanotrophs (e.g., pBba, SEQ ID NO:5) and a transcriptional terminator (e.g., rrnB_txn_terminator, SEQ ID NO:13), an origin of transfer (oriT), and an origin of replication for E. coli (pUC ori).

FIG. 17 is a schematic representation of an exemplary expression vector “Plasmid 10” comprising sequences encoding a replication initiation protein (trfA), an origin of vegetative replication (oriV) that is functional in methanotrophic bacteria, an origin of transfer (oriT), an origin of replication for E. coli (pUC ori), a lad repressor protein (lacI) operably linked to a constitutive promoter that is functional in methanotrophs (e.g., 30S ribosomal protein S16), and a kanamycin resistance gene (KanR). Plasmid 9 also comprises an expression cassette comprising sequences encoding a recombinase (e.g., Gam, Bet, Exo), which are operably linked to a Lad inducible methanol dehydrogenase (MDH) promoter (MP2, SEQ ID NO:3).

FIG. 18 is a schematic representation of an exemplary pUC-based gene disruption plasmid, RS15395-targeting “Plasmid 11”, comprising an integration polynucleotide cassette comprising sequences encoding spectinomycin resistance, which is flanked by a 5′ homology flank segment on one end and a 3′ homology flank segment, on the other end. The 5′ and 3′ homology flank segments are homologous to the 5′ upstream and 3′ downstream sequences of the methanotroph target gene RS15395. Plasmid 11 also comprises sequences encoding an origin of transfer (oriT), and an origin of replication for E. coli (pUC ori).

FIG. 19 is a schematic representation of an exemplary pUC-based gene disruption plasmid, MCA1474-targeting “Plasmid 12”, comprising an integration polynucleotide cassette comprising sequences encoding gentamicin resistance (gentR) and a donor molecule (e.g. Cas9) operably linked with a constitutive promoter functional in Methylococcus Bath, which are flanked by a PAM sequence, MCA1474 target sequence and 5′ homology flank segment on one end and a PAM sequence, MCA1474 target sequence and 3′ homology flank segment, on the other end. The 5′ and 3′ homology flank segments are homologous to the 5′ upstream and 3′ downstream sequences of the methanotroph target DNA cleavage site within the MCA1474 locus generated by the MCA1474-specific polynucleotide modification system. Plasmid 12 also comprises sequences encoding a MCA1474-targeting RNA (e.g., MCA01474 specific DNA targeting domain and a duplex forming region (aka. scaffold RNA)), which is operably linked to a constitutive promoter that is functional in methanotrophs (e.g., pBba, SEQ ID NO:5) and a transcriptional terminator (e.g., rrnB_txn_terminator, SEQ ID NO:13), an origin of transfer (oriT), and an origin of replication for E. coli (pUC ori).

### DETAILED DESCRIPTION

The CRISPR/Cas system was originally identified as an adaptive immune system that employs CRISPR RNA (crRNA)-guided Cas proteins to recognize target sites within the invader genome (known as protospacers) via base-pairing complementarity and then to cleave DNA within the protospacer sequences (see Horvath et al., Science 327:167-170; 2010). CRISPR/Cas systems are classified into three types (i.e., type I, type II, and type III) based on the sequence and structure of the Cas proteins (see, e.g., Makarova et al., Biol. Direct 6:38, 2011; Makarova et al., Nat. Rev. Microbiol. 9:467-77, 2011). The crRNA-guided surveillance complexes in types I and III need multiple Cas subunits (Sinkunas et al., EMBO J. 32:385-94, 2013; Zhang et al., Mol. Cell 45:303-313, 2012). However, type II systems require only Cas9 (Deltcheva et al., 2011. Nature 471:602-607, 2011; Sapranauskas et al., Nucleic Acids Res. 39:9275-9282, 2011). The type II system as a reduced system has been studied primarily in Streptococcus (see Deltcheva et al., 2011; Gasiunas et al., Proc. Natl. Acad. Sci. U.S.A. 109:E2579-E2586, 2012) and Neisseria (Zhang et al., Mol. Cell 50:488-503, 2013). The naturally occurring type II system requires at least three crucial components: an RNA-guided Cas9 nuclease, a crRNA, and a partially complementary trans-acting crRNA (tracrRNA) (see, e.g., Deltcheva et al., 2011; Gasiunas et al., 2012; Zhang et al., 2013).

Recently it was determined that segments of crRNA and tracrRNA sequences can be combined into a single guide RNA (sgRNA) (see, e.g., Jinek et al., Science 337:816-21, 2012). Further, the region of the guide RNA complementary to the target site can be altered or programed to target a desired sequence. As discussed in more detail herein, target specificity is determined by the guide RNA and a short motif associated with the target DNA, known as a protospacer adjacent motif (PAM).

The instant disclosure provides nucleic acid molecules that encode a site-specific polynucleotide modification system heterologous to methanotrophic bacteria, along with vectors and methods for using the same in order to genetically manipulate the methanotrophic bacteria at the genomic level. In particular embodiments, the instant disclosure provides compositions and methods for using a CRISPR/Cas system to genetically engineer methanotrophic bacteria having desired properties. For example, methanotrophic bacteria genetically modified according to the instant disclosure may be used to express a variety of high value products (e.g., proteins, metabolites, chemical compounds, or the like), particularly when controlled cultivation on a C₁ substrate is desired.

Prior to setting forth this disclosure in more detail, it may be helpful to an understanding thereof to provide definitions of certain terms to be used herein. Additional definitions are set forth throughout this disclosure.

In the present description, the term “about” means ±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

As used herein, the term “C₁ substrate” refers herein to any carbon containing molecule that lacks a carbon-carbon bond. Examples include methane, methanol, formaldehyde, formic acid, carbon monoxide, carbon dioxide, a methylated amine (such as, for example, methyl-, dimethyl-, and trimethylamine), methylated thiols, methyl halogens (e.g., bromomethane, chloromethane, iodomethane, dichloromethane), cyanide, or the like.

As used herein, the term “wild-type” or “native” as applied to a microorganism, polypeptide or polynucleotide means a microorganism, polypeptide, or polynucleotide found in nature.

As used herein, the term “endogenous” refers to a reference molecule or activity that is present in a parental or host methanotroph.

As used herein, “heterologous” nucleic acid molecule, construct or sequence refers to a nucleic acid molecule or portion of a nucleic acid molecule, or a construct containing such a nucleic acid molecule or fragment thereof, that is not native to a host cell or is a nucleic acid molecule with an altered expression as compared to the native expression level under similar conditions. For example, a heterologous regulatory element (e.g., promoter, enhancer) may be used to regulate expression of a native gene or nucleic acid molecule in a way that is different from the way a native gene or nucleic acid molecule is normally expressed in nature or in culture. In certain embodiments, heterologous nucleic acid molecules may not be endogenous to a host cell or host genome, but instead have been added to a host cell by conjugation, transformation, transfection, electroporation, or the like, wherein the added polynucleotide may integrate into the host genome or can exist as extra-chromosomal genetic material (e.g., as a plasmid or other self-replicating vector). In addition, “heterologous” can refer to an enzyme, protein or other activity that is different or altered from that found in a host cell, or is not native to a host cell but instead is encoded by a nucleic acid molecule introduced into the host cell. In certain embodiments, more than one heterologous nucleic acid molecules can be introduced into a host cell as separate nucleic acid molecules, as a polycistronic operon, as a single nucleic acid molecule encoding a fusion protein, or any combination thereof, and still be considered as more than one heterologous nucleic acid.

It is to be understood that when one or more heterologous nucleic acid molecules are included in a host microorganism, the one or more heterologous nucleic acid molecules may be referred to as an encoding nucleic acid molecule or as an enzymatic activity. It is also to be understood, as disclosed herein, that more than one heterologous nucleic acid molecule can be introduced into a host microorganism on different or the same vector as individually regulated expression constructs, as a polycistronic operon, as a single nucleic acid molecule encoding a fusion protein, or any combination thereof, and still be considered more than one heterologous nucleic acid molecule. Thus, the number of referenced heterologous nucleic acid molecules or enzymatic activities indicates the number of encoding nucleic acids or the number of enzymatic activities, not the number of separate vectors introduced into a host cell.

The term “homologous” or “homolog” refers to a molecule or activity found in or derived from a host cell, species or strain. For example, a heterologous nucleic acid molecule may be homologous to a native host cell gene, but may have an altered expression level or have a different sequence or both.

Recombinant DNA, molecular cloning, and gene expression techniques used in the present disclosure are known in the art and described in references, such as Sambrook et al., Molecular Cloning: A Laboratory Manual, 3^{rd} Ed., Cold Spring Harbor Laboratory, New York, 2001, and Ausubel et al., Current Protocols in Molecular Biology, John Wiley and Sons, Baltimore, MD, 1999.

As used herein, the term “chimeric” or “fusion” refers to any nucleic acid molecule or protein that is not endogenous and comprises sequences joined or linked together that are not normally found joined or linked together in nature. For example, a chimeric or fusion nucleic acid molecule may comprise regulatory sequences and coding sequences that are derived from different sources (which may be from the same organism, same organism but different species, from a different genus, or from a different domain (archaea, prokaryote, eukaryote)), or regulatory sequences and coding sequences that are derived from the same source but arranged in a manner different than that found in nature.

As used herein, the terms “nuclease” and “endonuclease” are used interchangeably to mean an enzyme that possesses catalytic activity for DNA cleavage.

As used herein, the term “cleavage” or “cleave” refers to the breakage of the covalent backbone of a nucleic acid molecule. Cleavage can be initiated by a variety of methods including, for example, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a targeting RNA and a modification polypeptide (e.g., Cas9 polypeptide) is used for targeted double-stranded DNA cleavage of a target DNA. The location in a target DNA where cleavage occurs is referred to herein as a “cleavage site.”

As used herein, the term “genetic modification” or “genetic engineering” refers to an engineered alteration to the genetic material (e.g., genome, plasmid, both, etc.) of a wild type or parental methanotroph, such as, for example, by introducing expressible nucleic acid molecules encoding proteins or engineering other nucleic acid molecule additions, deletions, substitutions, or other functional addition or disruption of a microorganism's genetic material. Exemplary genetic modifications include a knock out or deletion of an endogenous gene (for example, by insertion of an in-frame mutation into a gene), introducing a heterologous polynucleotide into a methanotroph in the form of a plasmid or vector or by integration of the heterologous polynucleotide into the chromosome of the methanotroph. Such genetic engineering can include, for example, introducing heterologous polynucleotides encoding a heterologous polypeptide or a polypeptide homologous to a polypeptide of the methanotroph host, or encoding functional polypeptide fragments thereof, or encoding fusion or chimeric molecules. Additionally, methanotrophs may be engineered to include, for example, polynucleotides containing non-coding regulatory regions that can alter expression of one or more genes or an operon. A genetic modification can silence, activate, or modulate (either increase or decrease) the expression or translation of an RNA encoding a polypeptide or fragment thereof or fusion polypeptide, or the activity of a polypeptide or fragment thereof or fusion polypeptide encoded by the DNA. Genetic modifications can include nucleic acid molecules encoding enzymes or functional fragments thereof or fusion polypeptides to confer a biochemical reaction capability that was present or not in a methanotroph, or can include genetically engineered nucleic acid molecules encoding modified enzymes or functional fragments thereof or fusion polypeptides that have an altered (improved or reduced) biochemical reaction capability when expressed by a methanotroph as compared to a parent methanotroph.

As used herein, the terms “non-naturally occurring” and “non-natural,” when used in reference to a microorganism, means that the microorganism has at least one genetic ally engineered modification that is not normally found in a naturally occurring wild-type or parent microorganism. The terms “non-naturally occurring” and “non-natural,” when used in reference to a polynucleotide or polypeptide, means that the polynucleotide or polypeptide has at least one genetically engineered modification that is not found in the naturally occurring polynucleotide or polypeptide, or a coding sequence of a polynucleotide is operably linked to a regulatory element in a construct or an orientation that does not occur in nature.

As used herein, the term “inactivating mutation” when used in the context of an endogenous gene refers to a substitution, deletion, insertion, or combinations thereof, of one or more nucleotides in the gene or endogenous regulatory element in the chromosome of methanotrophic bacteria that results in a significant decrease in activity. In some embodiments, the activity is less than 50%, 45%, 40%, 35%, 30%, 25%, 20%, 15%, 10%, 5%, or 1%, or is null or not detectable compared to the wild type or parental activity.

As used herein, “nucleic acid molecule,” also known as “polynucleotide,” refers to a polymeric compound comprised of covalently linked subunits called nucleotides. Nucleic acid molecules include polyribonucleic acid (RNA), polydeoxyribonucleic acid (DNA), either of which may be single or double stranded. DNA includes cDNA, genomic DNA, synthetic DNA, and semi-synthetic DNA.

As used herein, the terms “polypeptide” and “protein” are used interchangeably, and refer to a polymeric form of amino acids of any length, which can include coded amino acids, non-coded amino acids, chemically or biochemically modified or derivatized amino acids, and polypeptides having modified peptide backbones. The term “peptide” is defined the same as polypeptides, except that peptides are generally shorter than polypeptides and range in length from two to about 100 amino acids.

As used herein, the terms “coding sequence” or “coding region” or “CDS” are intended to refer to a DNA polynucleotide that is transcribed into RNA. A DNA polynucleotide may encode an RNA (mRNA) that is translated into one or more protein products, or a DNA polynucleotide may encode an RNA that is not translated into protein (e.g. tRNA, rRNA, siRNA, miRNA, guide RNA; also referred to as “functional RNA”). A “protein coding sequence” or a sequence that encodes a particular protein or polypeptide, refers to a nucleic acid molecule, when placed under the control of an appropriate regulatory element, that can be transcribed into mRNA (in the case of DNA), which mRNA can be translated into a polypeptide. Transcription, translation or both reactions can be carried out in vitro or in vivo. The boundaries of the coding sequence are generally determined by an open reading frame, which usually begins with a start codon (e.g., standard AUG, non-standard such as CUG).

The term “DNA construct” is used herein to refer to a DNA molecule comprising a vector and at least one polynucleotide insert. A DNA construct is usually generated for the purpose of expressing and/or propagating the insert(s), or for the construction of other recombinant polynucleotide sequences. The insert(s) may or may not be operably linked to a regulatory element (e.g., a promoter, operator).

As used herein, the term “expression cassette” refers to a DNA construct that contains a regulatory element operably linked to a nucleic acid molecule containing a coding sequence. A coding sequence contained in a nucleic acid molecule integrated into an expression cassette can be transcribed into an RNA, which can in turn be a functional RNA or translated into one or more polypeptides or fragments thereof. In certain embodiments, an expression cassette can comprise a nucleic acid molecule containing a coding sequence that can be transcribed into a functional RNA (e.g., a targeting RNA).

The term “vector” refers to a polynucleotide used as a vehicle to carry heterologous genetic material extrachromosomally, aid in transferring such material into another host cell, or aid in integrating such material into a host cell chromosome. In any of these embodiments, a vector can be self-replicating or replicated as part of a chromosome, and optionally express any nucleic acid molecule of interest inserted into or carried on the vector. In certain embodiments, a vector is a plasmid.

As used herein, the term “expression vector” refers to a DNA construct comprising an expression cassette.

The “percent identity” between two or more nucleic acid sequences or between two or more polypeptide sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more nucleic acid sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). Similarly, a comparison of sequences and determination of percent identity between two or more polypeptide sequences can be accomplished using a mathematical algorithm, such as ClustalW analysis (version W 1.8 available from European Bioinformatics Institute, Cambridge, UK), counting the number of identical matches in the alignment and dividing such number of identical matches by the length of the reference sequence, and using the following default ClustalW parameters to achieve slow/accurate pairwise optimal alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

As used herein, the term “fragment” refers to a portion of a polynucleotide or a portion of a polypeptide. A fragment of a protein or polypeptide, unless otherwise specified, retains the biological activity of the wild-type protein. For example, a fragment of a Cas9 protein retains the specified activity (e.g., interacting with targeting RNA or endonuclease activity). In some embodiments, a fragment of a polynucleotide encodes a fragment of a protein or polypeptide.

As used herein, “variant” is intended to mean a substantially similar sequence. For polynucleotides, a variant comprises a deletion, insertion, substitution or any combination thereof of one or more nucleotides at one or more internal sites within the wild-type or reference polynucleotide. For polynucleotides, conservative variants include those sequences that, because of the degeneracy of the genetic code, encode the amino acid sequence of one of the polypeptides as disclosed herein (e.g., Cas9). Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to wild-type or reference polynucleotide as determined by sequence alignment programs and parameters as described herein.

A “variant” protein or polypeptide is intended to mean a protein or polypeptide derived from a wild-type or reference protein or polypeptide by a deletion, insertion, substitution or any combination thereof of one or more amino acids at one or more internal sites in the reference protein or polypeptide. Variant polypeptides or proteins are biologically active—that is, they retain or continue to possess a desired biological activity, for example, at least one activity of the wild type or a reference protein. Such variants may result from, for example, genetic polymorphism or from human manipulation. In some embodiments, biologically active variants of a particular protein or polypeptide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the wild-type or reference protein as determined by sequence alignment programs and parameters as described herein. A biologically active variant of a polypeptide or protein as disclosed herein may differ from that polypeptide or protein by as few as one to about 15 amino acid residues, as few as one to about 10, as few as about 6 to about 10, as few as 5, as few as 4, 3, 2, or one amino acid residue.

By “hybridizable” or “complementary” or “substantially complementary” it is meant that a nucleic acid (e.g., RNA) comprises a sequence of nucleotides that enables it to non-covalently bind, i.e. form Watson-Crick base pairs and/or G/U base pairs, “anneal” or “hybridize,” to another nucleic acid in a sequence-specific, anti-parallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Generally, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides. Furthermore, the artisan of ordinary skill will recognize that the temperature and wash solution salt concentration may be adjusted as necessary according to factors such as length of the region of complementation and the degree of complementarity.

It is understood in the art that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid molecule to which they are targeted. For example, an antisense nucleic acid molecule in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol. 215:403-410, 1990; Zhang and Madden, Genome Res. 7:649-656, 1997) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math. 2:482-489, 1981).

As used herein, “homology-directed repair” or “HDR” refers to the specialized form of DNA repair that takes place, for example, during repair of double-strand breaks in cells. This process uses a homologous polynucleotide as a template to repair a cleaved “target DNA” molecule, which can lead to the integration of genetic information into the cleaved target sequence. In certain embodiments, a homologous polynucleotide is referred to as an “integration polynucleotide.” Homology-directed repair may result in an alteration of the sequence of the target molecule (e.g., insertion, deletion, mutation), if the integration polynucleotide differs from the target molecule and part or all of the sequence of the integration polynucleotide is incorporated into the target DNA. In some embodiments, an integration polynucleotide, a portion of the integration polynucleotide, a copy of the integration polynucleotide, or a portion of a copy of the integration polynucleotide integrates into a target DNA.

As used herein, the term “non-homologous end joining” or “NHEJ” is the repair of double-strand breaks in DNA by direct ligation of the break ends to one another without the use of a homologous template (in contrast to homology-directed repair, which requires a homologous sequence to guide repair). NHEJ often results in the loss (deletion) of nucleotides near the site of the double-strand break.

As used herein, the term “binding” refers to a non-covalent interaction between macromolecules (e.g., between proteins; between a protein and a nucleic acid molecule). While in a state of non-covalent interaction, the macromolecules are said to be “associated” or “interacting” or “binding” (e.g., when a molecule X is said to interact or associate with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. For example, binding can be between a DNA molecule and a protein, between an RNA molecule and a protein, between two or more proteins, or any combination thereof. For example, Cas9 can bind to both DNA and RNA.

As used herein, “codon optimization” refers to the alteration of codon sequence in genes or coding regions at the nucleic acid molecule level to reflect a more common codon usage of a host cell without altering the amino acid encoded by the codon. Codon optimization methods for maximal nucleic acid expression in a heterologous host have been previously described (see, e.g., Welch et al., PLoS One 4:e7002, 2009; Gustafsson et al., Trends Biotechnol. 22:346, 2004; Wu et al., Nucl. Acids Res. 35:D76, 2007; Villalobos et al., BMC Bioinformatics 7:285, 2006; U.S. Patent Publication Nos. US 2011/0111413 and US 2008/0292918; the methods of which are incorporated herein by reference in their entirety).

### A. Site-Specific Polynucleotide Modification System

The present disclosure provides nucleic acid molecules encoding a site-specific polynucleotide modification system, vectors, and methods for using the same to genetically modify the methanotrophic bacteria genome. In certain embodiments, the nucleic acid molecules encoding a site-specific polynucleotide modification system disclosed herein comprise a CRISPR/Cas system that is functional in methanotrophic bacteria. While the presence of the CRISPR/Cas system has been known in bacteria and archaea as an adaptive immunity mechanism (see, e.g., Xu et al., Appl Environ Microbiol., 80:1544-52, 2014), such a system has not been used in methanotrophic bacteria to make site-directed genetic modifications in their genome.

In certain aspects, provided herein are methods of altering the genome of methanotrophic bacteria, comprising culturing under conditions and for a time sufficient to allow methanotrophic bacteria containing heterologous nucleic acid molecules encoding a site-specific polynucleotide modification system to express the site-specific polynucleotide modification system; wherein the nucleic acid molecules encoding the site-specific polynucleotide modification system are operably linked to a regulatory element in a vector, the nucleic acid molecules comprising: (a) a first heterologous nucleic acid molecule encoding a modification polypeptide, wherein the modification polypeptide comprises a targeting RNA binding domain and a site-specific nuclease domain, and (b) a second heterologous nucleic acid molecule encoding a targeting RNA, wherein the targeting RNA comprises a duplex-forming region and a DNA-targeting domain; and wherein the expressed modification polypeptide and the expressed targeting RNA form a complex that binds to and cleaves a genomic target sequence of the methanotrophic bacteria, thereby site-specifically altering the genome of the methanotrophic bacteria.

The site-specific polynucleotide modification systems of this disclosure comprise a variety of components, including a modification polypeptide, a targeting RNA, and a target DNA. Furthermore, these components have various characteristics that allow them to interact in a particular way and facilitate the cleavage of a methanotrophic bacteria genome. Each of these components is further described herein.

(1) Modification Polypeptide

As used herein, the term “modification polypeptide” refers to a nuclease having a targeting RNA binding domain and a site-specific nuclease domain, wherein the modification polypeptide is an inactive nuclease until it interacts, associates, or complexes with a targeting RNA molecule, at which point the modification polypeptide becomes an RNA-guided, site-specific DNA nuclease. As used herein, a “modification polypeptide/RNA complex” refers to the RNA-guided DNA nuclease (e.g., Cas9 polypeptide) that is bound, interacting, associated or complexed with a targeting RNA. In some embodiments, a modification polypeptide/RNA complex is a Cas9 complex comprising a Cas9 polypeptide bound to or associated with a crRNA/tracrRNA duplex. In other embodiments, a Cas9 complex is a Cas9 polypeptide bound to or associated with an sgRNA. The specificity of the nuclease activity is influenced by a variety of factors, including (i) the level of base-pairing complementarity between a targeting RNA and its target DNA; and (ii) the protospacer adjacent motif (PAM) of the target DNA.

The terms “protospacer adjacent motif” or “PAM” are used interchangeably herein and refer to a short sequence ranging from about 2 nucleotides to about 5 nucleotides that are adjacent to or very near the 3′ end of the target DNA sequence recognized by a targeting RNA-modification polypeptide complex (e.g., sgRNA-Cas9 complex). A targeting RNA-modification polypeptide complex will not bind to or cleave a target DNA sequence unless it is followed by a requisite PAM for the modification polypeptide. In certain embodiments, a PAM is about 1, 2, 3, 4, 5, 6, 7, 8, 9, or 10 nucleotides downstream of a target DNA sequence cleavage site. The precise sequence and length requirement of a PAM for a modification polypeptide/RNA complex binding differs depending on the modification polypeptide used. For example, the Cas9 of S. pyogenes recognizes a 5′-NGG-3′ sequence, wherein N can be any nucleotide (Mali et al., Science 339:823, 2013). The S. thermophilus Cas9 systems recognize: 5′-NGGNG-3′ (Horvath and Barrangou, Science 327:167, 2010), 5′-NAGAAW-3′ or 5′-NNAAAAW-3′, wherein W is A or T (Deveau et al., J. Bacteriol. 190:1390, 2008; Fonfara et al., Nucl. Acids Res. 42:2577, 2013, respectively), and 5′-NNAGAAW-3′ (Cong et al., Science 339:819, 2013). Different S. mutans Cas9 systems can use 5′-NGG-3′ or 5′-NAAR-3′, wherein R is A or G (van der Ploeg et al., Microbiology 155:1966, 2009). In another example, S. aureus Cas9 systems can recognize 5′-NNGRRT-3′ (Ran et al., Nature 520:186-91, 2015). In yet another example, N. meningitidis Cas9 recognizes a 5′-NNNNGATT-3′ PAM sequence (Hou et al., Proc. Natl. Acad. Sci. U.S.A. 110:15644, 2013). Additional examples of PAM sequences and their respective cognate Cas9 polypeptides are described in U.S. Pat. Appl. Pub. No. US 2014/0068797, U.S. Pat. No. 8,697,359, and WO 2015/071474, which PAM sequences and Cas9 polypeptides are incorporated herein by reference in their entirety. Moreover, in vitro methods for characterization of PAM sequences and guide RNA requirements in newly discovered Cas9 proteins have also been described (Karvelis et al., Genome Biol. 16:253, 2015).

In certain embodiments, a modification polypeptide comprises a Cas9 polypeptide, or a homolog, ortholog, paralog, functional variant or functional fragment thereof. Wild-type Cas9 is a polypeptide that exhibits site-directed nuclease activity capable of cleaving DNA at a specific or target sequence defined by the region of complementarity between a targeting RNA and the target DNA. In some embodiments, a Cas9 polypeptide contains two nuclease domains, e.g., a His-Asn-His (HNH) nuclease domain and a RuvC-like nuclease domain. Cas9 proteins are also referred to as Csn1, Csx12, or a clustered regularly interspaced short palindromic repeat (CRISPR)-associated nuclease. CRISPR/Cas systems, including Cas9 proteins and variants thereof, are reviewed in Xu et al., Appl. Environ. Microbiol. 80:1544, 2014, of which the Cas9 proteins and variants thereof are incorporated herein by reference in their entirety. Cas9 also includes an engineered Cas9 endonuclease (e.g., modified, improved, chimeric) that retains its ability to cleave a target DNA. In some embodiments, an engineered Cas9 endonuclease is a Cas9 nuclease that has been engineered to modify its PAM recognition specificity. For example, Kleinstiver et al. disclose methods of modifying Cas9 to recognize alternative PAM sequences using structural information, bacterial selection-based directed evolution, and combinatorial design methods (Nature 523:481, 2015). In another example, PAM recognition specificity of Cas9 may be modified using molecular evolution techniques (Kleinstiver et al., Nat. Biotechnol. 33:1293, 2015). In other embodiments, an engineered Cas9 is a chimeric polypeptide comprising a Cas9 polypeptide fused to a zinc finger DNA-binding domain to enhance targeting precision (Bolukbasi et al., Nat. Methods 12:1150, 2015). In yet other embodiments, an engineered Cas9 protein is a Cas9 protein deletion mutant, which has been engineered to omit portions of the protein while still functioning as site-directed DNA nuclease (see, PCT Published Appl. No. WO 2015/077318). In yet further embodiments, an engineered Cas9 protein is a Cas9 chimeric polypeptide comprising an N-terminus of Neisseria meningitides Cas9 protein and C-terminus of Streptococcus thermophiles (see PCT Published Appl. No. WO2015/077318). In certain embodiments, Cas9 can induce cleavage in a nucleic acid molecule target, which can be either a double-stranded break or a single-stranded break.

Various different Cas9 polypeptides may be used in the methods provided herein to take advantage of differing enzymatic characteristics of the different Cas9 polypeptides, such as, for example, recognition of different PAM sequence preferences, having increased enzymatic activity, or having reduced enzymatic activity. Exemplary Cas9 polypeptides that can be used in the methods of the instant disclosure include Cas9 polypeptides from Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter (see, e.g., U.S. Patent Pub. No. US 2014/0186919, of which the Cas9 homologs, orthologs and associated PAM sequences are hereby incorporated by reference in their entirety). Additional Cas9 orthologs and variants and associated PAM sequences have been described in PCT Published Appl. No. WO 2015/071474, which Cas9 homologs, orthologs and associated PAM sequences are hereby incorporated by reference in their entirety.

In certain embodiments, the modification polypeptide is a Cas9 polypeptide from S. pyogenes (see GenBank Nos. AAK33936 or NP_269215), Streptococcus thermophilus (see GenBank No. YP_820832), Listeria innocua (see GenBank No. NP_472073), Staphylococcus aureus (GenBank No. WP_001573634.1), or Neisseria meningitidis (see GenBank No. YP_002342100). Plasmids harboring polynucleotides encoding Cas9 polypeptides are available from repository sources, such as Addgene (Cambridge, MA). Examples of such plasmids include pMJ806 (S. pyogenes Cas9), pMJ823 (L. innocua Cas9), pMJ824 (S. thermophiles Cas9), and pMJ839 (N. meningitides Cas9). In further embodiments, a Cas9 polypeptide is encoded by a first heterologous nucleic acid molecule of a site-specific polynucleotide modification system and the encoded Cas9 polypeptide has at least 80%, 85%, 90%, 90%, 91%, 92%, 93%, 94%, 95%, 96%, 97%, 98%, 99% or more sequence identity to a polypeptide sequence corresponding to any one of GenBank Nos. YP_820832, NP_472073, YP_002342100, AAK33936, WP_001573634.1, or NP_269215. In still further embodiments, a first heterologous nucleic acid molecule of a site-specific polynucleotide modification system comprises a polynucleotide sequence encoding a polypeptide comprising SEQ ID NO:1. In yet further embodiments, a first heterologous nucleic acid molecule of a site-specific polynucleotide modification system encodes a functional Cas9 polypeptide comprising a sequence having at least about 70%, about 75%, about 80%, about 85%, about 90%, about 91%, about 92%, about 93%, about 94%, about 95%, about 96%, about 97%, about 98%, about 99% or 100% sequence identity to SEQ ID NO:1. In particular embodiments, a nucleic acid molecule encoding a Cas9 polypeptide of a site-specific polynucleotide modification system is codon optimized for expression in a selected methanotrophic bacterium (e.g., Methylococcus capsulatus Bath or Methylosinus trichosporium OB3b).

(2) Targeting RNA

The term “targeting RNA” refers to an RNA molecule comprised of a DNA-targeting domain and a duplex forming region (also referred to as a “scaffold component” or “scaffold RNA”). As used herein, the term “duplex-forming region” refers to a portion of the targeting RNA that forms part of a double-stranded RNA duplex that binds to or interacts with a modification polypeptide (e.g., Cas9 polypeptide) to form a modification polypeptide/RNA complex. In certain embodiments, a duplex-forming region comprises a short palindromic repeat sequence. As used herein, “DNA-targeting domain” refers to a portion of the targeting RNA that is complementary to a target sequence located in a target DNA (i.e., complementary to one strand of the target DNA). The targeting RNA may have all functionalities on a single chain molecule (i.e., duplex-forming region, DNA-targeting domain in a single guide RNA (sgRNA)). Alternatively, a targeting RNA may be comprised of two molecules, a first RNA molecule and a second RNA molecule, wherein at least a portion of the first RNA molecule (DNA-targeting domain) and a portion of the second RNA molecule (duplex forming region) anneal through the duplex-forming region to form the targeting RNA.

In certain embodiments, a targeting RNA comprises a two polynucleotide chains that anneal through a duplex-forming region. A first RNA chain comprises a duplex-forming region and a DNA-targeting domain (referred to herein as a “DNA-specificity RNA” or “crRNA”). A second RNA chain comprises a duplex forming region complementary to the duplex-forming region of the first RNA chain (referred to herein as a “scaffold RNA” or “tracrRNA”). Downstream of the duplex-forming region of the second RNA chain (also referred to as “crRNA base-pairing region”), the second RNA chain may comprise additional nucleotides that may form additional RNA structures (e.g., hairpin loop). While sequences downstream of the duplex-forming region on the tracrRNA (“tracrRNA tail”) are not required for site-specific Cas9 cleavage (Jinek et al., Science 337:816, 2012), a tracrRNA tail may enhance Cas9 cleavage activity (Hsu et al., Nat. Biotechnol. 31:827, 2013). The DNA-specificity RNA and scaffold RNA form a duplex molecule (i.e., the targeting RNA) that interacts with a modification polypeptide (e.g., a Cas9 polypeptide) and targets the modification polypeptide/RNA complex to a specific target DNA determined by the DNA-targeting domain within the DNA-specificity RNA and the PAM on the target DNA. In particular embodiments, a crRNA and a tracrRNA form a duplex that is capable of interacting with a Cas9 polypeptide and guiding the Cas9/RNA complex to a specific target DNA due to the DNA-targeting domain of the crRNA and a PAM on the target DNA. The exact sequence of a given crRNA or tracrRNA molecule is dependent upon the Cas9 polypeptide and the region of DNA that is targeted. In certain embodiments, a crRNA, tracrRNA, or both are derived from naturally occurring sequences. In other embodiments, a crRNA, tracrRNA, or both are non-naturally occurring (e.g., synthetic). Pre-designed or custom synthetic crRNA and tracrRNA reagents have been described (Randar et al., Proc. Natl. Acad. Sci. U.S.A. 112:E7110, 2015) and are commercially available (e.g., Dharmacon, Lafayette, CO).

Appropriate naturally occurring duplex-forming regions of crRNAs and tracrRNAs can be determined by taking into account the source species and base-pairing for the dsRNA duplex of the protein-binding domain when determining appropriate duplex-forming regions (see, e.g., PCT Published Appl. No. WO 2015/071474; Fontara et al., Nucleic Acids Res. 42:2577, 2014). Non-cognate pairs are also contemplated. In some embodiments, a non-cognate crRNA and tracrRNA pair is from or derived from homologous or orthologous Cas9 endonucleases, wherein the Cas9 polypeptides share at least 80% identity over at least 80% of their amino acid sequences.

Accordingly, in some embodiments, methanotrophic bacteria comprise an endogenous tracrRNA that forms a duplex with an exogenous or heterologous target crRNA and thereby interacts with a modification polypeptide (e.g., Cas9 polypeptide). In other embodiments, a nucleic acid molecule encoding an exogenous or heterologous tracrRNA is introduced into the methanotrophic bacteria containing an exogenous or heterologous crRNA, wherein the crRNA comprises a duplex-forming region complementary to the exogenous or heterologous tracrRNA being introduced.

In certain embodiments, a targeting RNA comprises a single molecule, which comprises a DNA-targeting domain and a duplex-forming region (scaffold component) in a single chain RNA molecule. As used herein, the terms “sgRNA,” “gRNA,” “chimeric RNA,” and “chiRNA” are used interchangeably and refer to a single-molecule targeting RNA. The duplex-forming region of the single chain RNA comprises self-complementary sequence that can form a duplex structure (e.g., hairpin loop) that facilitates binding to a modification polypeptide (e.g., Cas9 polypeptide), and this modification polypeptide/RNA complex will specifically bind to a target DNA (through the DNA-targeting domain of the targeting RNA in the complex) and subsequently cleave the target DNA. In certain embodiments, a sgRNA may comprise additional nucleotides that may form additional RNA structures (e.g., hairpin loop) downstream of the duplex-forming region.

Methods and sequences for designing and making sgRNAs are described in, for example, Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and WO 2015/071474, which methods and sequences are incorporated herein by reference in their entirety. Methods of designing sgRNA to maximize activity and minimize off-target effects of CRISPR-Cas are also described in, for example, Doench et al., Nat. Biotechnol. 32:1262, 2014; Doench et al., Nat. Biotechnol. 34:184, 2016; which methods are incorporated herein by reference in their entirety. In some embodiments, an sgRNA interacts with a Cas9 polypeptide and targets the Cas9 to a specific target DNA sequence determined by a DNA-targeting domain of the targeting RNA and a PAM of the target DNA. The exact sequence of a given sgRNA molecule is dependent upon the Cas9 polypeptide and the DNA sequence that is targeted.

In some embodiments, a DNA-targeting domain of a targeting RNA comprises at least about 5, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 75, or about 80 nucleotides in length. In some embodiments, a DNA-targeting domain is less than about 80, about 75, about 50, about 45, about 40, about 35, about 30, about 25, about 20, about 15, about 12, about 11, about 10 nucleotides in length. In certain embodiments, a DNA-targeting domain of a targeting RNA is about 20 nucleotides in length. In particular embodiments, a DNA-targeting domain of a targeting RNA is about is 16 nucleotides, about 17 nucleotides, about 18 nucleotides or about 19 nucleotides in length.

In some embodiments, a duplex forming region forms a duplex length at least about 10, about 11, about 12, about 13, about 14, about 15, about 16, about 17, about 18, about 19, about 20, about 21, about 22, about 23, about 24, or about 25 nucleotides in length. In some embodiments, the duplex forming region in the two RNA chains or self-complementary duplex forming region of the sgRNA has at least 50%, 55%, 60%, 65%, 70%, 75%, 80%, 85%, 90%, 95%, 97.5%, 99% complementarity.

The ability of a DNA-targeting domain of a targeting RNA to direct sequence-specific binding of a modification polypeptide/RNA complex to a target DNA may be assessed by any suitable assay. For example, a targeting RNA, comprising the DNA-targeting domain to be tested, and a modification polypeptide (e.g., Cas9) may be provided to a host cell having the corresponding target sequence, such as by transformation with vectors encoding the components as described herein, followed by an assessment of preferential cleavage within the target sequence, such as by using a Surveyor® assay (IDT, Coralville, Iowa). Similarly, cleavage of a target DNA sequence may be evaluated in a test tube by providing a target DNA, components of a modification polypeptide complex (e.g., Cas9 complex), including a targeting RNA comprising a DNA-targeting domain to be tested and a control DNA-targeting domain different from the test DNA-targeting domain, and comparing binding or rate of cleavage of the target DNA between the test and control DNA-targeting domain reactions.

In some embodiments, a nucleic acid molecule encoding a targeting RNA further comprises a polynucleotide sequence at the 5′-end, 3′-end, or both ends that provides for additional features. In some embodiments, a nucleic acid molecule encoding a targeting RNA further encodes a self-cleaving ribozyme sequence at the 5′-end of the targeting RNA, at the 3′-end of the targeting RNA, or both. As used here, the term “self-cleaving ribozyme” refers to an RNA structural motif that can cleave itself into two separate ribonucleotides in a sequence-specific manner. In some embodiments, a self-cleaving ribozyme has self-cleavage activity against sequences 5′ to its own sequence, e.g., as with a hepatitis delta ribozyme. In some embodiments, a self-cleaving ribozyme may be used to separate a targeting RNA from another sequence. For example, a ribozyme may self-cleave the expressed targeting RNA (e.g., gRNA) at the 5′-end, 3′-end, or both ends of the targeting RNA. Exemplary ribozyme sequences are provided herein and include, for example, U.S. Pat. Appl. Pub. No. US 2005/0158741, the ribozyme sequences of which are incorporated herein by reference in their entirety. Other examples of self-cleaving ribozymes may include, for example, hepatitis delta virus (HDV), glmS, hammerhead, hairpin, and Varkud satellite (VS) ribozymes. Sequences of hepatitis delta ribozymes have been disclosed (Been and Wickham, Eur. J. Biochem. 247:741, 1997; Chadalavada et al., RNA 13:2189, 2007). In some embodiments, a self-cleaving ribozyme is codon optimized for expression in a selected methanotrophic bacterium (e.g., Methylococcus capsulatus Bath or Methylosinus trichosporium OB3b). In some embodiments, a self-cleaving ribozyme comprises a polynucleotide sequence corresponding to SEQ ID NO:8 or SEQ ID NO:9.

In some embodiments, a nucleic acid molecule encoding a targeting RNA further comprises a transcriptional terminator. As used herein, the term “transcriptional terminator” refers to a section of nucleic acid sequence that marks the end of a coding sequence, gene, or operon in a nucleic acid sequence during transcription. In some embodiments, a transcriptional terminator provides secondary structures in the transcribed RNA that trigger processes which release the RNA from the transcriptional complex. In certain embodiments, a transcriptional terminator encodes an RNA sequence that forms a secondary structure that interacts with the transcription complex. In other embodiments, a transcriptional terminator encodes an RNA sequence that forms a secondary structure that terminates transcription by recruiting termination factors. In certain embodiments, the transcriptional terminator comprises a polynucleotide sequence corresponding to any one of SEQ ID NOS:7 and 13-17.

It is further contemplated that in some instances it may be desirable to use more than one targeting RNAs. Accordingly, in some embodiments, the methods described herein comprise the introduction of two or more, three or more, or four or more targeting RNAs. In certain embodiments, each of the plurality of targeting RNAs can target different or overlapping target sites on the same target DNA. In further embodiments, the plurality of targeting RNAs can target different or overlapping target sites on different target DNAs. In some embodiments, at least two target RNAs target at least two target sites that are at least about 10 nucleotides apart, at least about 15 nucleotides apart, at least about 20 nucleotides apart, at least about 25 nucleotides apart, at least about 30 nucleotides apart, at least about 35 nucleotides apart, at least about 40 nucleotides apart, at least about 45 nucleotides apart, at least about 50 nucleotides apart, at least about 75 nucleotides apart, at least about 100 nucleotides apart, at least about 150 nucleotides apart, at least about 200 nucleotides apart, at least about 250 nucleotides apart, at least about 300 nucleotides apart, at least about 400 nucleotides apart, at least about 500 nucleotides apart, at least about 600 nucleotides apart, at least about 700 nucleotides apart, at least about 800 nucleotides apart, at least about 900 nucleotides apart, at least about 1,000 nucleotides apart, or more.

(3) Target DNA

A “target DNA,” as used herein, refers to a DNA polynucleotide that comprises a “target site” or “target sequence.” The terms “target site” and “target sequence” are used interchangeably herein to refer to a nucleic acid sequence present in a target DNA to which a targeting RNA will bind, provided sufficient conditions for binding exist. Suitable DNA-RNA binding conditions include physiological conditions normally present in a cell (e.g., in a methanotrophic bacterium). In certain embodiments, a target DNA comprises or is located within a protein encoding sequence (e.g., gene), a regulatory element, or both.

In general, a DNA-targeting domain of a targeting RNA is any portion of the targeting RNA having sufficient complementarity with a target DNA to be able to specifically hybridize or anneal with that target DNA and thereby direct site-specific binding of a modification polypeptide/RNA complex with a target DNA and subsequent cleavage of the target DNA. In some embodiments, the degree of complementarity between a target DNA and its corresponding DNA-targeting domain of a targeting RNA, when aligned using a suitable alignment algorithm, is at least about 50%, about 55%, about 60%, about 65%, about 75%, about 80%, about 85%, about 90%, about 95%, about 96%, about 97%, about 98%, about 99%, or more. Suitable algorithms for aligning sequences include, for example, the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, CA), SOAP (available at soap.genomics.org.cn), and Maq (available at maq.sourceforge.net).

In some embodiments, a target DNA site comprises at least about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 75, or about 80 nucleotides in length. In some embodiments, a target DNA is less than about 80, about 75, about 50, about 45, about 40, about 35, about 30, about 25, about 20, about 15, about 12, about 11, about 10 nucleotides in length. In certain embodiments, a target DNA site is about 20 nucleotides in length. In particular embodiments, a target DNA site comprises about 16 nucleotides, about 17 nucleotides, about 18 nucleotides, about 19 nucleotides in length, or about 20 nucleotides in length.

A target DNA or target sequence therein can be readily identified from genomic sequences of methanotrophs in databases such as, for example, the integrated microbial genomes (IMG) system provided by the Joint Genome Institute (img.jgi.doe.gov). The genomes of many methanotrophs have been sequenced (see, e.g., Ward et al. PLoS Biol. 2:e303, 2004; Methylococcus capsulatus Bath, GenBank No. AE017282.2; Methylomonas methanica MC09, GenBank No. CP002738.1; Methylomicrobium album BG8, GenBank No. CM001475.1; Methylomicrobium alcaliphilum, GenBank No. F0082060.1; Methylobacterium extorquens PA1, GenBank No. CP000908.1; Methylobacterium extorquens CM4, GenBank No. CP001298.1; Methylobacterium sp. 4-46, GenBank No. CP000943.1; Methylobacterium populi BJ001, GenBank No. CP001029.1; Methylobacterium radiotolerans JCM 2831, GenBank No. CP001001.1; Methylocystis sp. SC2, GenBank No. HE956757.1; Methylocella silvestris BL2, GenBank No. CP001280.1; Methylobacterium nodulans ORS 2060, GenBank No. CP001349.1; Methylibium petroleiphilum PM1, GenBank No. CP000555.1). In addition, it is well established in the art how to sequence a bacterial genome, if needed (e.g., sequencing systems available from Illumina, Roche 454, or the like). Accordingly, targeting RNAs and integration polynucleotides can be readily designed as described herein to target a region of interest in a methanotroph genome (see, e.g., PCT Published Appl. No. WO 2015/065964).

(4) Other Components: Integration Polynucleotide, Recombinase

Compositions and methods for use of a site-specific polynucleotide modification system as described herein to alter the genome of methanotrophic bacteria may include altering the DNA near the cleavage site in the target DNA produced by a modification polypeptide/RNA complex. In certain embodiments, a site-specific polynucleotide modification system further comprises an integration polynucleotide to modify a methanotrophic bacteria DNA to include a point mutation, a frameshift mutation, a deletion, a substitution, an insertion, or any combination thereof.

As used herein, the term “integration polynucleotide” refers to a nucleic acid molecule that comprises a nucleic acid sequence to be inserted at the cleavage site of a target DNA created by a modification polypeptide/RNA complex (e.g., Cas9/gRNA complex). The integration polynucleotide comprises sufficient sequence identity to a target DNA at or nearby the cleavage site generated by the site-specific polynucleotide modification system to support homology-directed repair between the integration polynucleotide and the target DNA to which it bears homology.

In some embodiments, an integration polynucleotide comprises at least about 70%, about 75%, about 80%, about 85%, about 90%, about 91%, about 92%, about 93%, about 94%, about 95%, about 96%, about 97%, about 98%, about 99%, or 100% identity with the nucleotide sequences flanking the cleavage site of a target DNA. In further embodiments, a sequence flanking the cleavage site of a target DNA is within about 50 nucleotides, within about 30 nucleotides, within about 15 nucleotides, within about 10 nucleotides, within about 5 nucleotides, or immediately flanking the cleavage site of the target DNA. Approximately 20, 25, 50, 100, or 200 nucleotides, or more than 200 nucleotides (or any integral value between 10 and 200 nucleotides, or more), of sequence identity between an integration polynucleotide and a target DNA sequence (e.g., genomic) can support homology-directed repair.

In certain embodiments, an integration polynucleotide comprises a donor molecule, which may encode for certain desired functionalities (e.g., reporter molecule, enzymatic activity) or create a knockout mutation of an endogenous gene or operon. A “donor molecule” refers to a nucleic acid molecule of interest to be inserted into a host DNA at a modification polypeptide/RNA complex (e.g., Cas9/gRNA complex) cleavage site that modifies or replaces an endogenous host coding sequence (e.g., gene), regulatory element (e.g., promoter), other DNA region of interest, or any combination thereof. Donor molecules can range in length from, for example, 1 nucleotide to about 5,000 nucleotides, from about 10 nucleotides to about 1,000 nucleotides, from about 50 nucleotides to about 750 nucleotides, from about 100 nucleotides to about 500 nucleotides, or from about 250 nucleotides to about 5,000 nucleotides, or more. In some embodiments, an integration polynucleotide comprises two or more, three or more, four or more, or five or more donor molecules.

A donor molecule may contain at least one or more single base changes, insertions, deletions, inversions or rearrangements with respect to a target DNA that the donor molecule will modify or replace. Accordingly, in certain embodiments, a donor molecule comprises a nucleic acid molecule having 1, 2, 3, 4, 5, 6, 7, 8, 9, 10, 11, 12, 13, 14, 15, 16, 17, 18, 19, 20, or up to about 500 nucleotides that are additions, deletions, or substitutions from the target DNA sequence. In other embodiments, an integration polynucleotide adds or deletes from a host DNA one nucleotide or more, about 10 nucleotides or more, about 50 nucleotides or more, about 100 nucleotides or more, about 250 nucleotides or more, about 500 nucleotides or more, about 1,000 nucleotides, or more.

In some embodiments, a donor molecule comprises a mutation that modifies the target sequence such that the target sequence can no longer be cleaved by the cognate targeting RNA-Cas9 complex. In some embodiments, the mutation is a silent mutation that changes the nucleic acid sequence, but not the amino acid sequence of an encoded polypeptide. In some embodiments, a donor molecule does not include a PAM sequence that is present in a target DNA and recognized by the cognate targeting RNA-Cas9 complex.

In further embodiments, an integration polynucleotide comprises a non-homologous sequence (e.g., an insert) flanked by two regions of homology to a target DNA (referred to herein as 5′-homology flank and 3′-homology flank segments), such that homology-directed repair between the target DNA region and the two flanking sequences of the integration polynucleotide results in insertion of the non-homologous sequence at the cleavage site. The terms “5′-homology flank” and “3′-homology flank” refer to segments of DNA located either 5′ or 3′ of the non-homologous sequence, respectively, and have sufficient sequence identity to a target genomic sequence flanking the cleavage site to support homologous recombination between the integration polynucleotide and the target genomic sequence to which it bears homology. A 5′-homology flank or a 3′-homology flank having sufficient homology to support homologous recombination will have at least 60%, 70%, 80%, 90%, 95%, 98%, 99%, or 100% sequence identity with nucleotide sequences flanking the cleavage site of the target DNA.

A sequence flanking the cleavage site of a target DNA may be within about 50 nucleotides, within about 30 nucleotides, within about 15 nucleotides, within about 10 nucleotides, within about 5 nucleotides, or immediately flanking the cleavage site of the target DNA. Approximately 20, 21, 22, 23, 24, 25, 30, 35, 40, 45, 50, 55, 60, 65, 70, 75, 80, 85, 90, 95, 100, 125, 150, 175, 200, 250, 300, 350, 400, 450, 500 nucleotides or more, of sequence homology between a 5′-homology flank or 3′-homology flank and a target DNA sequence (or any integral value between 20 and 500 nucleotides) can support homologous recombination.

In certain embodiments, a vector comprising an integration polynucleotide comprises a repeat region that is homologous to a repeat region adjacent to or in the vicinity of the methanotroph host cell genome target site. In further embodiments, the repeat region is interior to a 5′-homology flank segment or a 3′-homology flank segment within an integration polynucleotide. Integration of the integration polynucleotide, including the repeat region, into the methanotroph genome target site, allows for a convenient subsequent loop out of the integration polynucleotide, thus providing for a markerless integration/deletion system in methanotrophic bacteria.

In further embodiments, an integration polynucleotide comprises a nucleic acid molecule that encodes a desired protein, polypeptide or activity. In certain embodiments, a desired protein or polypeptide encoded by the integration polynucleotide comprises a heterologous polypeptide, an exogenous polypeptide, an endogenous polypeptide, or any combination thereof.

As used herein, the term “selectable marker” means a phenotypic trait, encoded by a genetic element that can be detected under appropriate conditions. For example, an antibiotic resistance marker serves as a useful selectable marker since it enables detection of cells that are resistant to the antibiotic when the cells are grown in or on media containing that particular antibiotic. Thus, exemplary nucleic acid molecules that encode desired proteins or polypeptides that can be inserted into a host methanotroph genome and expressed include selectable markers, such as antibiotic resistance cassettes, fluorescent proteins, enzymes, or any combination thereof. Representative antibiotic resistance cassettes include cassettes providing resistance to kanamycin, ampicillin, spectinomycin, tetracycline, chloramphenicol, neomycin, hygromycin, zeocin or any combination thereof.

Additional exemplary desired polypeptides include reporter proteins, such as green fluorescent protein (GFP), red fluorescent protein (RFP), blue fluorescent protein (BFP), yellow fluorescent protein (YFP), orange fluorescent protein (OFP); proteins that enable increased production of desired chemicals or metabolites (e.g., an amino acid biosynthesis enzyme (such as lysine biosynthesis enzymes, threonine biosynthesis enzymes, methionine biosynthesis enzymes, cysteine biosynthesis enzymes), isoprene synthase, crotonase, crotonyl CoA thioesterase, 4-oxalocrotonate decarboxylase, fatty acid converting enzymes (such as fatty acyl-CoA reductase, a fatty alcohol forming acyl-ACP reductase, a carboxylic acid reductase), fatty acid elongation pathway enzymes (such as β-ketoacyl-CoA synthase, a β-ketoacy-CoA reductase, a β-hydroxy acyl-CoA dehydratase, an enoyl-CoA reductase), carbohydrate biosynthesis enzyme (such as glucan synthase) and lactate dehydrogenase; and antibiotic resistance proteins.

In any of the aforementioned aspects, embodiments of an encoded desired protein may be an enzyme, a fluorescent protein (e.g., green fluorescent protein (GFP), GFP green variant Dasher, a therapeutic protein (e.g., ligand, receptor), a vaccine antigen, an anti-parasitic protein, or the like. In some embodiments, an encoded desired protein is a metabolic pathway enzyme involved in the biosynthesis of a metabolite (e.g., amino acid). As used herein, metabolites refer to intermediates and products of metabolism, including primary metabolites (compound directly involved in normal growth, development, and reproduction of an organism or cell) and secondary metabolites (organic compounds not directly involved in normal growth, development, or reproduction of an organism or cell but have important ecological function). Examples of metabolites that may be produced in the modified methanotrophic bacteria described herein include alcohols, amino acids, nucleotides, antioxidants, organic acids, polyols, antibiotics, pigments, sugars, vitamins or any combination thereof. Desired chemicals or metabolites include, for example, isoprene, lactate, and amino acids (e.g., L-lysine, L-valine, L-tryptophan, and L-methionine). Host cells containing such recombinant polynucleotides are useful for the production of desired products (e.g., lactate, isoprene, propylene).

In some examples, a polynucleotide encoding a desired protein is a polynucleotide encoding lactate dehydrogenase (LDH). Methanotrophic bacteria that are genetically modified to express or over-express a lactate dehydrogenase and are capable of converting a carbon feedstock (e.g., methane) into lactate have been described in PCT Published Appl. No. WO 2014/205145, which recombinant polynucleotides and constructs are incorporated herein by reference in their entirety.

In some examples, a polynucleotide encoding a desired protein is a polynucleotide encoding a multi-carbon substrate utilization pathway component. Examples of multi-carbon substrate utilization pathway components that may be expressed include glycerol kinase, glycerol-3-phosphate dehydrogenase, glycerol uptake facilitator, or any combination thereof. Methanotrophic bacteria that are genetically modified to express or over-express a multi-carbon substrate utilization pathway component and are capable of growing on a multi-carbon feed stock as a primary or sole carbon source have been described in PCT Published Appl. No. WO2014/066670, which recombinant polynucleotides and constructs are incorporated herein by reference in their entirety.

In other examples, a polynucleotide encoding a desired protein is a polynucleotide encoding a propylene synthesis pathway enzyme, for example, crotonase, crotonyl CoA thioesterase, 4-oxalocrotonate decarboxylase, or any combination thereof. Methanotrophic bacteria that are genetically modified to be capable of converting carbon feedstock into propylene have been described in PCT Published Appl. No. WO 2014/047209, which recombinant polynucleotides and constructs thereof are incorporated herein by reference in their entirety.

In still other examples, a polynucleotide encoding a desired protein is a polynucleotide encoding an isoprene synthesis pathway enzyme (e.g., isoprene synthase (IspS)). Methanotrophic bacteria that are genetically modified to express or over-express isoprene synthase and are capable of converting carbon feedstock into isoprene have been described in PCT Published Appl. No. WO 2014/138419, which recombinant polynucleotides and constructs thereof are incorporated herein by reference in their entirety.

In more examples, a polynucleotide encoding a desired protein is a polynucleotide encoding a fatty acid converting enzyme, for example a fatty acyl-CoA reductase, a fatty alcohol forming acyl-ACP reductase, a carboxylic acid reductase, or any combination thereof. Methanotrophic bacteria that are genetically modified to express or over-express fatty alcohols, hydroxyl fatty acids, or dicarboxylic acids from carbon feedstock have been described in PCT Published Appl. No. WO 2014/074886, which recombinant polynucleotides and constructs thereof are incorporated herein by reference in their entirety.

In more examples, a polynucleotide encoding a desired protein is a polynucleotide encoding a methane monooxygenase that is stable in the presence of chemical or environmental stress. Methanotrophic bacteria that are genetically modified to express or over-express a methane monooxygenase that is stable in the presence of chemical or environmental stress and have at least one alcohol dehydrogenase enzyme inactivated, and are useful for producing alcohols and epoxides, have been described in PCT Published Appl. No. WO 2014/062703 (which recombinant polynucleotides and constructs thereof are incorporated herein by reference in their entirety).

In yet more examples, a polynucleotide encoding a desired protein is a polynucleotide encoding a fatty acid elongation pathway enzyme, for example, a 3-ketoacyl-CoA synthase, a (3-ketoacy-CoA reductase, a 3-hydroxy acyl-CoA dehydratase, an enoyl-CoA reductase, or any combination thereof. Methanotrophic bacteria that are genetically modified to express or over-express very long chain fatty acids, very long chain fatty alcohols, very long chain ketones, very long chain fatty ester waxes, and very long chain alkanes have been described in PCT Published Appl. No. WO 2015/175809, which recombinant polynucleotides and constructs thereof are incorporated herein by reference in their entirety.

In further examples, a polynucleotide encoding a desired protein is a polynucleotide encoding an amino acid biosynthesis enzyme. For example, a lysine biosynthesis enzyme may be a lysine-sensitive aspartokinase III (lysC), an aspartate kinase, an aspartate-semialdehyde dehydrogenase (asd), a dihydrodipicolinate synthase (dapA), a dihydrodipicolinate reductase (dapB), a 2,3,4,5-tetrahydropyridine-2,6-carboxylate N-succinyltransferase (dapD), an acetylornithine/succinyldiaminopimelateaminotransferase (argD), a succinyl-diaminopimelate desuccinylase (dapE), a succinyldiaminopimelate transaminase, a diaminopimelate epimerase (dapF), a diaminopimelate dicarboxylase (lysA), or the like. Exemplary tryptophan biosynthesis enzymes include a chorismate-pyruvate lyase (ubiC), an anthranilate synthase component I (trpE), an anthranilate synthase component II (trpG), an anthranilate phosphoribosyltransferase (trpD), a phosphoribosylanthranilate isomerase (trpC), a tryptophan biosynthesis protein (trpC), an N-(5′phosphoribosyl) anthranilate isomerase (trpF), an indole-3-glycerol phosphate synthase, a tryptophan synthase alpha chain (trpA), a tryptophan synthase beta chain (trpB), or the like. Representative methionine biosynthesis enzyme include a homoserine O-succinyltransferase (metA), a cystathionine gamma-synthase (metB), a protein MalY, a cystathionine beta-lyase (metC), a B12-dependent methionine synthase (metH), a 5-methyltetrahydropteroyltriglutamate-homocysteine S-methyltransferase (metE), or the like. Exemplary cysteine biosynthesis enzymes include a serine acetyltransferase (CysE), a cysteine synthase A, a cysteine synthase B, or the like. Representative threonine biosynthesis enzymes include an aspartate transaminase, a PLP-dependent aminotransferase, an aspartate aminotransferase, an aspartate kinase, an aspartate-semialdehyde dehydrogenase, a homoserine dehydrogenase, a homoserine kinase, a threonine synthase, or the like. Methanotrophic bacteria that are genetically modified to express or over-express amino acids have been described in PCT Published Appl. No. WO 2015/109265, which recombinant polynucleotides and constructs thereof are incorporated herein by reference in their entirety.

In further examples, a polynucleotide encoding a desired protein is a polynucleotide encoding a carbohydrate biosynthesis enzyme, such as, for example, pyruvate carboxylase, a phosphoenolpyruvate carboxykinase, an enolase, a phosphoglycerate mutase, a phosphoglycerate kinase, a glyceraldehyde-3-phosphate dehydrogenase, a Type A aldolase, a fructose 1,6-bisphosphatase, a phosphofructokinase, a phosphoglucose isomerase, a hexokinase, a glucose-6-phosphate, glucose-1-phosphate adenyltransferase, a glycogen synthase, glucan synthase (e.g., a β-1,3-glucan synthase), or the like. Methanotrophic bacteria that are genetically modified to express or over-express carbohydrates have been described in PCT Published Appl. No. WO 2015/109257, filed on Jan. 16, 2015, which recombinant polynucleotides and constructs thereof are incorporated herein by reference in their entirety.

In further embodiments, an integration polynucleotide is used to modify a host regulatory element. For example, an integration polynucleotide can modify a host promoter, thereby modulating the expression of a cognate gene or operon. In particular embodiments, a modified host regulatory element results in upregulation or overexpression of a host gene or operon. In other embodiments, a modified host regulatory element results in down-regulation or inactivation of expression of a host gene or operon. For example, an integration polynucleotide can comprise a regulatory element disposed between a 5′-homology flank and a 3′-homology flank, wherein the regulatory element may be heterologous, non-homologous or a modified endogenous regulatory element. In particular embodiments, the regulatory element can comprise a native promoter, a constitutive promoter, an inducible promoter, a chimeric promoter, an inactive promoter, or the like.

It is further contemplated that it may be desirable to introduce more than one integration polynucleotide into a methanotrophic bacterium. Accordingly, in some embodiments, at least 2, at least about 3, at least about 4, or at least about 5 integration polynucleotides are introduced into a methanotroph. Each integration polynucleotide may be introduced simultaneously or sequentially.

In yet further embodiments, a method of genetically modifying methanotrophic bacteria further comprises introducing into the methanotrophic bacteria a nucleic acid molecule encoding a recombinase. As used herein, a “recombinase” refers to a protein or catalytic domain thereof, or protein system that provides a measurable increase in the recombination frequency between two or more polynucleotides that are at least partially homologous (e.g., integration polynucleotide and target DNA).

An exemplary recombinase system is the bacteriophage lambda Red system. The lambda Red system includes three genes: gamma, beta, and exo, whose products are called Gam, Bet, and Exo, respectively (Murphy et al., J. Bacteriol. 180:2063, 1998). Double strand breaks in DNA are the initiation sites for recombination (Thaler et al., J. Mol. Biol. 195:75, 1987). It is thought that Gam prevents the degradation of linear dsDNA by host nucleases (such as RecBCD and SbcCD in E. coli); Exo degrades dsDNA in a 5′ to 3′ manner, leaving single-stranded DNA in the recessed regions; and Bet binds to the single-stranded regions produced by Exo and facilitates recombination by promoting annealing to the homologous genomic target site (see, e.g., Sawitzke et al. Methods Enzymol. 421:171, 2007; Mosberg et al., Genetics 186:791, 2010). A lambda Red recombinase system comprising Beta, Exo, and Gam is described in, for example, U.S. Pat. No. 7,144,734, which system and components are hereby incorporated by reference in their entirety.

The lambda Red recombinase system has been used to successfully modify the genomes of several species of bacteria, including E. coli (Datsenko et al., PNAS 97:6640, 2000), Salmonella enterica (Husseiny et al., Infect Immun. 73:1598, 2005), Yersinia pseudotuberculosis (Derbise et al., FEMS Immunol Med Microbiol. 38:113, 2003), Shigella flexneri (Beloin et al., Mol Microbiol. 47:825, 2003), Serratia marcescens (Rossi et al., Mol Microbiol. 48:1467, 2003), Pseudomonas aeruginosa (Lesic et al., BMC Mol Biol. 9:20, 2008), and Vibrio cholerae (Yamamoto et al., Gene. 438:57, 2009). In some embodiments, a nucleic acid molecule encoding a recombinase is a lambda Red recombinase. In certain embodiments, a nucleic acid encoding a recombinase comprises a nucleic acid molecule encoding Bet, or functional fragments or variants thereof, operably linked a regulatory element (e.g., a promoter). In further embodiments, a nucleic acid molecule encoding a recombinase comprises a nucleic acid molecule encoding Exo, Gam, or both, operably linked to at least one regulatory element. In still other embodiments, a nucleic acid molecule encoding a recombinase comprises a nucleic acid molecule encoding Bet, Exo, and Gam, wherein Bet, Exo and Gam are arranged in a polycistronic operon. In particular embodiments, a nucleic acid molecule encoding a recombinase comprises any one of SEQ ID NOS:10, 11, 12, or combinations thereof.

Another exemplary recombinase system is Rac prophage RecE/RecT system (Zhang et al., Nat. Genet. 20:123, 1998). RecE is a 5′-3′ exonuclease and RecT is a ssDNA-binding protein that promotes ssDNA annealing, strand transfer, and strand invasion in vitro (Kushner et al., Proc. Natl. Acad. Sci. USA 68:824, 1971; Joseph and Kolodner, J. Biol. Chem. 258:10411, 1983; Clark et al., J. Bacteriol. 175:7673, 1993; Hall et al., J. Bacteriol. 175:277, 1993; Hall and Kolodner, Proc. Natl. Acad. Sci. USA 91:3205, 1994; Noirot and Kolodner, J. Biol. Chem. 273:12274, 1998). The RecE/RecT recombinase system has been used to modify various targets, including plasmids, episomes, and the E. coli chromosome (see, Zhang et al., Nat. Genet. 20:123, 1998; Muyrers et al., Genes Dev. 14:1971, 2000).

In certain embodiments, a nucleic acid molecule encoding a recombinase is a Rac recombinase. In some embodiments, a nucleic acid molecule encoding a recombinase comprises a nucleic acid molecule encoding RecE or a functional fragment or variant thereof, RecT or a functional fragment or variant thereof, or both, operably linked to at least one regulatory element. In further embodiments, a nucleic acid molecule encoding a recombinase comprises a nucleic acid molecule encoding RecE and RecT, wherein RecE and RecT are arranged in a polycistronic operon.

In yet another example, a recombinase system may comprise a RecA recombinase. RecA catalyzes ATP-driven homologous pairing and strand exchange of DNA molecules necessary for DNA recombinational repair in bacteria. In certain embodiments, a nucleic acid encoding a recombinase comprises a nucleic acid molecule encoding RecA, or a functional fragment or variant thereof, operably linked a regulatory element (e.g., a promoter).

In certain embodiments, a recombinase may be fusion protein comprising a nuclease-inactivated Cas9 and a recombinase catalytic domain (see, U.S. Patent Appl. Pub. No. US 2015/0071898, which fusion proteins and methods of use are hereby incorporated by reference in their entirety). Fusion proteins comprising a nuclease-inactivated Cas9 and a recombinase catalytic domain are capable of binding and recombining DNA at any selected site, e.g., sites specified by a targeting RNA (e.g., sgRNA). For example, a targeting RNA provided by a site-specific polynucleotide modification system described herein may be used to direct site-specific cleavage of a target DNA by a modification polypeptide (e.g., Cas9) and site-specific recombination at the target DNA cleavage site with a fusion protein comprising a nuclease-inactivated Cas9 and a recombinase catalytic domain.

In certain embodiments, an integration polynucleotide or portion thereof (e.g., a donor molecule) is codon optimized for expression in a selected methanotrophic bacterium (e.g., Methylococcus capsulatus Bath or Methylosinus trichosporium OB3b).

### B. Vectors

In the embodiments described herein, nucleic acid molecules encoding a site-specific polynucleotide modification system or other components can be contained within one or more vectors, which can be used, for example, to deliver the site-specific polynucleotide modification system or other components to methanotrophic bacteria. Exemplary vectors include a plasmid, a cosmid, a phage, a virus, a bacterial artificial chromosome (BAC), a yeast artificial chromosome (YAC), or the like. A vector may contain one or more of the following elements: origin of replication, regulatory element (e.g., promoter, operator, transcriptional terminator), gene encoding antibiotic resistance, or the like.

A vector may comprise a regulatory element (including, for example, a promoter, operator, ribosome binding sequence) operably linked to one or more coding sequences for components of the site-specific polynucleotide modification system or other components as described herein. In any of the embodiments disclosed herein, nucleic acid molecules encoding a site-specific polynucleotide modification system or other components may be contained in a vector and operatively linked to an appropriate regulatory element (e.g., promoter) to direct RNA synthesis. In some embodiments, a recombinant nucleic acid molecule is operatively linked to a promoter. The promoter may be constitutive, leaky, or inducible, and native or non-native (e.g., exogenous, heterologous) to the methanotrophic bacteria employed. Examples of vectors for use in methanotrophic bacteria are described in PCT Published Appl. No. WO 2015/195972 (which vectors are hereby incorporated by reference in their entirety).

As used herein, the term “regulatory element” refers to any segment or sequence of DNA that functions as a promoter (e.g., native, exogenous, chimeric), operator, enhancer, leader, ribosome binding site, transcription terminator, or any combination thereof, or any other regulatory control mechanism of the associated DNA sequence. Regulatory elements can include hybrid regulatory regions comprising mixtures of parts of regulatory elements from different sources. A regulatory element that is operably linked to a coding sequence requires at least a promoter sequence.

Examples of such regulatory elements suitable for use in the compositions and methods of the present disclosure include a pyruvate decarboxylase (PDC) promoter, a deoxyxylulose phosphate synthase (DXS) promoter, a methanol dehydrogenase promoter (MDH) (such as, for example, the promoter in the upstream intergenic region of the mxaF gene from Methylococcus capsulatus Bath (Acc. No. MCA 0779) or the MDH promoter from M. extorquens (see Springer et al., FEMS Microbiol. Lett. 160:119, 1998), a hexulose 6-phosphate synthase promoter (HPS), a ribosomal protein S16 promoter, a serine phosphoenolpyruvate carboxylase promoter, a T5 promoter, Trc promoter, a promoter for PHA synthesis (Foellner et al., Appl. Microbiol. Biotechnol. 40:2384, 1993), a pyruvate decarboxylase promoter (Tokuhiro et al., Appl. Biochem. Biotechnol. 131:795, 2006), the lac operon Plac promoter (Toyama et al., Microbiol. 143:595, 1997), a hybrid promoter such as Ptrc (Brosius et al., Gene 27:161, 1984), a moxF promoter from Methylomonas 16a (e.g., SEQ ID NO:6), promoters identified from native plasmids in methylotrophs, methanotrophs, or the like.

As used herein, the term “promoter” refers to a region of DNA that initiates transcription of a coding sequence. Promoter sequences are located in the 5′ region near or adjacent to the transcription initiation site. RNA polymerase and transcription factors bind to the promoter sequence and initiate transcription. Promoter sequences define the direction of transcription and indicate which DNA strand will be transcribed; this strand is known as the sense strand. A promoter that is “functional in methanotrophic bacteria” is capable of initiating gene transcription in methanotrophic bacteria, and is optionally also capable of initiating gene transcription in non-methanotrophic bacteria. Any promoter sequence that is functional in methanotrophic bacteria may be included in the heterologous polynucleotides provided herein.

A “constitutive promoter” is a promoter that causes a coding sequence to be expressed under most culture conditions. Exemplary constitutive promoter sequences that are functional in methanotrophic bacteria include heterologous or endogenous promoters such as an MDH promoter, a ribosomal protein S16 promoter, a hexulose 6-phosphate synthase promoter, moxF promoter, and a Trc promoter, as well as synthetic promoters such as pBba (SEQ ID NO:5).

A “regulated promoter” or an “inducible promoter” is a promoter that is regulated, becoming active in response to a specific stimulus. An inducible promoter may be bound by a repressor. The binding of the repressor to the promoter may be inhibited by another agent, resulting in gene expression. Exemplary inducible promoters include a promoter of the lac operon and those in a tetracycline inducible promoter system, heat shock inducible promoter system, metal-responsive promoter system, nitrate inducible promoter system, light inducible promoter system, and ecdysone inducible promoter system.

In addition, an inducible promoter may be a non-inducible (e.g., constitutive) promoter operably linked to sequences that confer the property of inducibility. For example, an IPTG-inducible promoter may be engineered by linking a lacO control sequence with a natural methanotroph promoter, such as the MDH promoter or a variant thereof (e.g., SEQ ID NO:2 linked to a lacO sequence to create SEQ ID NO:3). Accordingly, in some embodiments, methods and microorganisms disclosed herein may further comprise a polynucleotide that encodes a lac repressor protein (Lad) (see, e.g., Oehler et al., EMBO J. 9:973, 1990). In certain embodiments, the inducible promoter may be a sodium benzoate inducible promoter (SEQ ID NO:4), which is controlled by the BenR activator. The BenR activator is encoded by a benR gene, which can be modified to incorporate codons favorable for expression in a methanotroph. IPTG inducible and sodium benzoate inducible promoters for use in methanotrophic bacteria are also described in PCT Published Appl. No. WO 2015/195972 (which promoters are hereby incorporated by reference in their entirety).

As used herein, the term “ribosomal binding sequence” refers to a DNA sequence encoding a 5′-untranslated sequence (“5′-UTS”) of an mRNA molecule that comprises a sequence encoding a ribosomal binding site and optionally a sequence encoding an RBS linker or a portion of an RBS linker as described herein. A ribosomal binding site (also called the “Shine-Dalgarno sequence” or “SD sequence”) is where the 30S ribosome small subunit binds first on mRNA and promotes efficient and accurate translation of mRNA. It is generally located a short distance upstream of a start codon (e.g., AUG, GUG, CUG), and is typically purine-rich. For example, a common consensus sequence among many bacterial SD sequences is AGGAGG, which is located a few or up to about 10 nucleotides upstream of a start codon. The sequence between a ribosomal binding site and a start codon on a prokaryotic mRNA is referred to as an “RBS linker.”

As used herein, the term “native MDH ribosomal binding sequence” refers to a naturally occurring MDH ribosomal binding sequence from a methanotrophic bacterium. A “modified MDH ribosomal binding sequence” refers to a ribosomal binding sequence that is different from a native MDH ribosomal binding sequence at one or more nucleotides, such as 1, 2, 3, 4, 5, 6, or more nucleotides. Exemplary native and modified MDH ribosomal binding sequences are provided in PCT Publication WO 2015/195972, of which the ribosomal binding sequences are hereby incorporated by reference in their entirety.

In certain embodiments, a regulatory element that is operably linked to a nucleic acid molecule encoding a site-specific polynucleotide modification system or other components (such as a modification polypeptide, a targeting RNA, a recombinase, an integration polynucleotide, or the like) comprises a promoter that is functional in methanotrophic bacteria. In further embodiments, a regulatory element is a promoter corresponding to the polynucleotide of SEQ ID NO.:2. In yet further embodiments, a regulatory element is a promoter corresponding to the polynucleotide of SEQ ID NO:6. In other embodiments, a regulatory element is an inducible promoter corresponding to the polynucleotide of SEQ ID NO:3. In still other embodiments, the inducible promoter may be a sodium benzoate inducible promoter (SEQ ID NO:4), which is controlled by the BenR activator. The BenR activator is encoded by a benR gene, which can be modified to incorporate codons favorable for expression in a particular host methanotroph. In yet other embodiments, a regulatory element is operably linked to a nucleic acid molecule encoding a targeting RNA, wherein the regulatory element is a promoter comprising a polynucleotide of SEQ ID NO:5 or SEQ ID NO:6.

As used herein, the term “operably linked” refers to a configuration in which a regulatory element (e.g., promoter, transcriptional terminator) is appropriately placed at a position relative to the coding sequence of a nucleic acid molecule such that the regulatory element influences the expression of the coding sequence. The transcript may be, for example, a functional RNA or an mRNA that is translated into a polypeptide.

In some embodiments, a vector may further encode for a selectable marker (e.g., antibiotic resistance) or a counter-selectable marker. As used herein, a “counter-selectable marker” refers to a nucleic acid molecule that encodes a polypeptide that promotes the death of the microorganism in which it is expressed. An exemplary counter-selectable marker for use in the compositions, methods and systems herein is SacB.

In further embodiments, a vector comprising any of the aforementioned nucleic acid molecules encoding a site-specific polynucleotide modification system or other components may comprise an origin of replication that is non-functional in methanotrophic bacteria, such as pUC- or pBR-based plasmids. In other embodiments, a vector may comprise a temperature sensitive origin of replication.

In particular aspects, provided herein are methanotrophic bacteria containing a vector, wherein the vector comprises: (a) a first heterologous nucleic acid molecule encoding a modification polypeptide operably linked to a regulatory element; (b) a second heterologous nucleic acid molecule encoding a targeting RNA operably linked to a regulatory element; and optionally (c) a third heterologous nucleic acid molecule comprising an integration polynucleotide. The first, second and third heterologous nucleic acid molecules may be on the same vector, on two different vectors, or on three different vectors. In addition, expression of the one or more of the first, second and third heterologous nucleic acid molecules that are located on the same vector may be controlled by the same or different regulatory elements.

In certain aspects, a first heterologous nucleic acid molecule encoding a modification polypeptide (e.g., Cas9 polypeptide) and a second heterologous nucleic acid molecule encoding a targeting RNA (e.g., sgRNA) are contained in the same vector. In certain embodiments, a first heterologous nucleic acid molecule is operably linked to a promoter that is functional in methanotrophic bacteria. In some embodiments, a promoter linked to the first heterologous nucleic acid molecule may be an inducible promoter, such as a natural methanotroph promoter (e.g., MDH promoter or a variant linked to a lacO sequence (see, SEQ ID NO:3)) or a sodium benzoate inducible promoter (SEQ ID NO:4). In embodiments where an inducible promoter is used, a vector may further comprise a nucleic acid molecule encoding a cognate repressor protein or inducer protein for the inducible promoter system (e.g., Lad for a promoter linked to lacO sequence; BenR for a sodium benzoate inducible promoter). In some embodiments, a repressor protein for an inducible promoter system is operably linked to a constitutive promoter that is functional in methanotrophic bacteria (e.g., ribosomal protein S16 promoter, MDH promoter, moxF promoter, Trc promoter, hexulose 6-phosphate promoter, pBba promoter, etc.). A vector may also comprise a pUC origin of replication, an origin of transfer, an origin of vegetative replication, a nucleic acid molecule encoding TrfA, and a nucleic acid encoding an antibiotic marker (e.g., kanamycin), which are all optionally operably linked to the same promoter as the repressor protein if present (e.g., Lad).

In certain embodiments, a second heterologous nucleic acid molecule is operably linked to a promoter that is functional in methanotrophic bacteria. In some embodiments, a promoter is SEQ ID NO:5 or SEQ ID NO:6. In some embodiments, a second heterologous nucleic acid molecule is also operably linked to a transcriptional terminator. In further embodiments, a transcriptional terminator is SEQ ID NO.:7, 13, 14, 15, 16, or 17. In yet further embodiments, a second heterologous nucleic acid molecule may be flanked by a self-cleaving ribozyme on its 5′-end, on its 3′end, or both. A self-cleaving ribozyme may be a hepatitis delta virus (HDV), glmS, hammerhead, hairpin, Varkud satellite (VS) ribozyme, or a combination of two ribozymes selected therefrom. In further embodiments, a self-cleaving ribozyme is SEQ ID NO:8 or SEQ ID NO:9.

In certain embodiments, a vector comprising a first heterologous nucleic acid and a second heterologous nucleic acid may further comprise a fourth heterologous nucleic acid molecule encoding a recombinase. A recombinase may comprise Lambda recombinase comprising Exo, Bet, Gam, or any combination thereof, a RecA recombinase, or a Rac recombinase comprising RecE, RecT, or both. In some embodiments, the first heterologous nucleic acid molecule and fourth heterologous nucleic acid molecule are operably linked to the same regulatory element (e.g., promoter). In some embodiments, the first heterologous nucleic acid molecule and fourth heterologous nucleic acid molecule are arranged in a polycistronic operon.

In embodiments where a first heterologous nucleic acid molecule encoding a modification polypeptide (e.g., Cas9 polypeptide) and a second heterologous nucleic acid molecule encoding a targeting RNA (e.g., sgRNA) are contained in a first vector, a third nucleic acid molecule comprising an integration polynucleotide may be in the same vector, or in a second vector. In certain embodiments, an integration polynucleotide comprises a 5′ homology flank segment, a donor molecule, and a 3′ homology flank segment. An integration polynucleotide may be a selectable marker protein, a reporter protein, a metabolic pathway enzyme, or a combination thereof.

In some aspects, the efficiency of homologous recombination of an insertion polynucleotide into the methanogen chromosome by a site-specific polynucleotide modification system described herein can be increased by having the integration polynucleotide excised from a vector after the vector has been introduced into the host methanogen. Accordingly, an integration polynucleotide may further comprise a target sequence and PAM sequence at its 5′-end (e.g., upstream of a 5′ homology flank segment) and at its 3′-end (e.g., downstream of a 3′ homology flank segment), wherein the target sequences are complementary to the DNA targeting domain of the targeting RNA in the first vector and the PAM sequences are recognized by the modification polypeptide in the first vector. Therefore, the modification polypeptide and targeting RNA encoded by the first vector will form a complex and perform site-specific cleavage of a target DNA (e.g., methanotroph genome) and also perform site-specific cleavage of the second vector comprising the integration polynucleotide to release the integration polynucleotide as a linear molecule, thereby improving introduction of the integration polynucleotide at the cleavage site of the methanotroph genome target DNA by homologous recombination.

In certain embodiments, a second vector comprising an integration polynucleotide may further comprise a gene encoding counter-selectable marker (e.g., SacB). The counter-selectable marker is optionally not located in the same part of the vector as the donor molecule, i.e., the donor molecule is separated from the counter-selectable marker on each end by the 5′ homology flank segment and 3′ homology flank segment. In those embodiments where the integration polynucleotide is on a second vector, separated from a first heterologous nucleic acid molecule encoding a modification polypeptide (e.g., Cas9 polypeptide) and a second heterologous nucleic acid molecule encoding a targeting RNA (e.g., sgRNA), the second vector may lack a functional origin of replication for the host methanotrophic bacteria (e.g., OriV) or possess a conditional origin of replication (e.g., temperature sensitive). Non-functional or reduced function origin of replication favors selection for chromosomal integration of the integration polynucleotide, as the integration polynucleotide sequence is lost as part of the non-replicative vector if it is not integrated into the host chromosome.

In other aspects, a first heterologous nucleic acid molecule encoding a modification polypeptide (e.g., Cas9 polypeptide) and a second heterologous nucleic acid molecule encoding a targeting RNA (e.g., sgRNA) are contained in different vectors, e.g., a first vector and second vector, respectively. In certain embodiments, a first vector comprises the first heterologous nucleic acid molecule operably linked to a promoter that is functional in methanotrophic bacteria. In some embodiments, a promoter linked to the first heterologous nucleic acid molecule may be an inducible promoter, such as a natural methanotroph promoter (e.g., MDH promoter or a variant linked to a lacO sequence (see, SEQ ID NO:3)) or a sodium benzoate inducible promoter (SEQ ID NO:4). In embodiments where an inducible promoter is used, the first vector may further comprise a nucleic acid molecule encoding a cognate repressor protein or inducer protein for the inducible promoter system (e.g., Lad for a promoter linked to lacO sequence; BenR for a sodium benzoate inducible promoter). In some embodiments, a repressor protein for an inducible promoter system is operably linked to a constitutive promoter that is functional in methanotrophic bacteria (e.g., ribosomal protein S16 promoter). A vector may also comprise a pUC origin of replication, an origin of transfer, an origin of vegetative replication, a nucleic acid molecule encoding TrfA, and a nucleic acid encoding an antibiotic marker (e.g., kanamycin), which are all optionally operably linked to the same promoter as the repressor protein if present (e.g., Lad). In certain embodiments, a first vector comprising a first heterologous nucleic acid may further comprise a fourth heterologous nucleic acid molecule encoding a recombinase. A recombinase may comprise Lambda recombinase comprising Exo, Bet, Gam, or any combination thereof, a RecA recombinase, or a Rac recombinase comprising RecE, RecT, or both. In some embodiments, the first heterologous nucleic acid molecule and fourth heterologous nucleic acid molecule are operably linked to the same regulatory element (e.g., promoter). In some embodiments, the first heterologous nucleic acid molecule and fourth heterologous nucleic acid molecule are arranged in a polycistronic operon.

In certain embodiments, a second vector comprising the second heterologous nucleic acid molecule encoding a targeting RNA is operably linked to a promoter that is functional in methanotrophic bacteria. In some embodiments, the promoter is SEQ ID NO:5 or SEQ ID NO:6. In some embodiments, a second heterologous nucleic acid molecule is also operably linked to a transcriptional terminator. In further embodiments, the transcriptional terminator is SEQ ID NO:7, 13, 14, 15, 16, or 17. In yet further embodiments, a second heterologous nucleic acid molecule may be flanked by a self-cleaving ribozyme on its 5′-end, on its 3′end, or both. A self-cleaving ribozyme may be a hepatitis delta virus (HDV), glmS, hammerhead, hairpin, Varkud satellite (VS) ribozyme, or a combination of two ribozymes selected therefrom. In further embodiments, a self-cleaving ribozyme is SEQ ID NO:8 or SEQ ID NO:9. A second vector may optionally comprise a third nucleic acid molecule comprising an integration polynucleotide. In certain embodiments, an integration polynucleotide comprises a 5′ homology flank segment, a donor molecule, and a 3′ homology flank segment. An integration polynucleotide may be a selectable marker protein, a reporter protein, a metabolic pathway enzyme, or a combination thereof. In some embodiments, an integration polynucleotide may further comprise a target sequence and PAM sequence at its 5′-end (e.g., upstream of a 5′ homology flank segment) and at its 3′-end (e.g., downstream of a 3′ homology flank segment), wherein the target sequences are complementary to the DNA targeting domain of the targeting RNA in the second vector and the PAM sequences are recognized by the modification polypeptide in the first vector. In certain embodiments, the second vector may further comprise a gene encoding counter-selectable marker (e.g., SacB). The counter-selectable marker is optionally not located in the same part of the vector as the donor molecule, i.e., the donor molecule is separated from the counter-selectable marker on each end by the 5′ homology flank segment and 3′ homology flank segment. The second vector may also comprise an pUC origin of replication, an origin of transfer, a nucleic acid molecule encoding TrfA, and preferably lacks a functional origin of replication for the host methanotrophic bacteria (e.g., OriV) or possesses a conditional origin of replication (e.g., temperature sensitive).

In certain embodiments, a second nucleic acid molecule encoding a targeting RNA is contained in a separate vector from the first nucleic acid molecule encoding a modification polypeptide and a fourth nucleic acid molecule encoding a recombinase. In some embodiments, the second nucleic acid molecule is operably linked to a regulatory element, such as a promoter, a transcriptional terminator, or both. In some embodiments, the second nucleic acid molecule further encodes a self-cleaving ribozyme sequence at the 5′-end, 3′-end, or both ends of the targeting RNA. In some embodiments, the vector comprising the second nucleic acid molecule further comprises a donor molecule (e.g., antibiotic marker, reporter protein, metabolic pathway enzyme). In further embodiments, the donor molecule is flanked by a 5′-homology flank, a 3′-homology flank, or both.

### C. Methanotrophic Bacteria

The methods and nucleic acids of the present disclosure may be used to genetically modify the genomic DNA of methanotrophic bacteria to impart or exhibit desired phenotypes. For example, the methanotrophic bacteria may be engineered to express or overexpress an endogenous or exogenous desired protein or to attenuate expression of an undesired endogenous protein. The present disclosure also provides methanotrophic bacteria host cells that comprise a site-specific polynucleotide modification system or other components as provided herein.

The term “parental” or “host” refers herein to methanotrophic bacteria that are an ancestor of a genetically modified or recombinant methanotroph of the present disclosure. A parental methanotrophic bacterium may be a wild-type methanotrophic bacterium, or may be an altered or mutated form of wild-type methanotrophic bacteria.

Methanotrophs have the ability to oxidize methane as a carbon and energy source. Methanotrophic bacteria are classified into three groups based on their carbon assimilation pathways and internal membrane structure: type I (gamma proteobacteria), type II (alpha proteobacteria, and type X (gamma proteobacteria). Type I methanotrophs use the ribulose monophosphate (RuMP) pathway for carbon assimilation whereas type II methanotrophs use the serine pathway. Type X methanotrophs use the RuMP pathway but also express low levels of enzymes of the serine pathway. Methanotrophic bacteria include obligate methanotrophs, which can only utilize C₁ substrates for carbon and energy sources, and facultative methanotrophs, which naturally have the ability to utilize some multi-carbon substrates as a sole carbon and energy source.

As used herein, the term “methylotroph” or “methylotrophic bacteria” refers to any bacteria capable of oxidizing organic compounds that do not contain carbon-carbon bonds. In certain embodiments, a methylotrophic bacterium may be a methanotroph. For example, “methanotrophic bacteria” refers to any methylotrophic bacteria that have the ability to oxidize methane as it primary source of carbon and energy. In certain other embodiments, the methylotrophic bacterium is an “obligate methylotrophic bacterium,” which refers to bacteria that are limited to the use of C₁ substrates for the generation of energy.

As used herein, the term “methanotroph” or “methanotrophic bacterium” or “methanotrophic bacteria” refers to methylotrophic bacteria capable of utilizing C₁ substrates, such as methane, natural gas or unconventional natural gas, as its primary or sole carbon and energy source. In addition, methanotrophic bacteria include “obligate methanotrophic bacteria” that can only utilize C₁ substrates (e.g., methane) for carbon and energy sources, and do not utilize organic compounds that contain carbon-carbon bonds (i.e., multicarbon-containing compounds) as a source of carbon and energy. Also included are “facultative methanotrophic bacteria” that are naturally able to use, in addition to C₁ substrates (e.g., methane), multi-carbon substrates, such as acetate, pyruvate, succinate, malate, or ethanol, as their carbon and energy source.

Methanotrophic bacteria are grouped into several genera, including Methylomonas, Methylobacter, Methylococcus, Methylocystis, Methylosinus, Methylomicrobium, Methanomonas, and Methylocella.

Methanotrophic bacteria include obligate methanotrophs and facultative methanotrophs. Facultative methanotrophs include some species of Methylocella, Methylocystis, and Methylocapsa (e.g., Methylocella silvestris, Methylocella palustris, Methylocella tundrae, Methylocystis daltona strain SB2, Methylocystis bryophila, Methylocapsa aurea KYG), and Methylobacterium organophilum (ATCC 27,886).

Exemplary methanotrophic bacteria species include: Methylococcus capsulatus Bath strain, Methylomonas 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus (NRRL B-11,201), Methylobacterium organophilum (ATCC 27,886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylacidiphilum fumariolicum, Methyloacida kamchatkensis, Methylibium petroleiphilum, and Methylomicrobium alcaliphilum.

In certain embodiments, methanotrophic bacteria of the present disclosure may be either an aerobic methanotroph or an anaerobic methanotroph. In particular embodiments, methanotrophic bacteria of the present disclosure are aerobic methanotrophs. In further embodiments, a host cell is a Methylococcus (e.g., Methylococcus capsulatus, including the strain Methylococcus capsulatus Bath) or Methylosinus (e.g., Methlosinus trichosporium, including the strain Methlosinus trichosporium OB3b).

In certain embodiment, provided herein are methods of genetically modifying methanotrophic bacteria, comprising introducing into the methanotrophic bacteria: (a) a first nucleic acid molecule encoding a modification polypeptide operably linked to a regulatory element; (b) a second nucleic acid molecule encoding a targeting RNA operably linked to a regulatory element; and optionally (c) a third nucleic acid molecule comprising an integration polynucleotide. In certain embodiments, any of the aforementioned nucleic acid molecules encoding one or more components of a site-specific polynucleotide modification system or other associated components, as well as any of the aforementioned vectors, are introduced into any of the aforementioned methanotrophic bacteria. As used herein, the term “introduced” or “introducing” in the context of inserting a nucleic acid molecule into a cell means transfected, transduced, transformed, electroporated, or introduction by conjugation (collectively “transformed”), wherein the nucleic acid molecule is incorporated into the genome of the cell, is extra-genomic, is on an episomal plasmid, or any combination thereof.

As used herein, the term “transformation” refers to the process of transferring a nucleic acid molecule (e.g., exogenous or heterologous nucleic acid molecule) into a host cell, which includes all methods of introducing polynucleotides into cells (such as transformation, transfection, transduction, electroporation, introduction by conjugation, or the like). The transformed host cell may carry the exogenous or heterologous nucleic acid molecule extra-chromosomally or the nucleic acid molecule may integrate into the chromosome. Integration into a host genome and self-replicating vectors generally result in genetically stable inheritance of the transformed nucleic acid molecule. Host cells containing the transformed nucleic acids are referred to as “modified,” “recombinant,” “non-naturally occurring,” “genetically engineered,” “transformed” or “transgenic” cells (e.g., bacteria).

Bacterial conjugation, which refers to a particular type of transformation involving direct contact of donor and recipient cells, is frequently used for the transfer of nucleic acids into methanotrophic bacteria. Bacterial conjugation involves mixing “donor” and “recipient” cells together in close contact with each other. Conjugation occurs by formation of cytoplasmic connections between donor and recipient bacteria, with unidirectional transfer of newly synthesized donor nucleic acid molecules into the recipient cells. A recipient in a conjugation reaction is any cell that can accept nucleic acids through horizontal transfer from a donor bacterium. A donor in a conjugation reaction is a bacterium that contains a conjugative plasmid or mobilized plasmid. The physical transfer of the donor plasmid can occur through a self-transmissible plasmid or with the assistance of a “helper” plasmid. Conjugations involving methanotrophic bacteria have been previously described in Stolyar et al., Mikrobiologiya 64:686, 1995; Motoyama et al., Appl. Micro. Biotech. 42:67, 1994; Lloyd et al., Arch. Microbiol. 171:364, 1999; PCT Pub. No. WO 02/18617; and Ali et al., Microbiol. 152:2931, 2006, the methods of which are incorporated by reference herein.

In addition, electroporation of C₁ metabolizing bacteria, such as methylotrophs or methanotrophs, has been previously described in, for example, Toyama et al., FEMS Microbiol. Lett. 166:1, 1998 (Methylobacterium extorquens); Kim and Wood, Appl. Microbiol. Biotechnol. 48:105, 1997 (Methylophilus methylotrophus AS1); Yoshida et al., Biotechnol. Lett. 23:787, 2001 (Methylobacillus sp. strain 12S); and U.S. Pat. Appl. Pub. No. US 2008/0026005 (Methylobacterium extorquens).

In some embodiments, the present disclosure provides a modified methanotrophic bacteria, comprising a first heterologous nucleic acid molecule encoding a modification polypeptide (e.g., Cas9 polypeptide) operably linked to a regulatory element; a second heterologous nucleic acid molecule encoding a targeting RNA operably linked to a regulatory element; and optionally a third heterologous nucleic acid molecule comprising an integration polynucleotide.

In further embodiments, the present disclosure provides a modified methanotrophic bacteria, wherein modified methanotrophic bacteria comprise at least one recombinant or heterologous polynucleotide integrated into their genome that encodes a desired protein, modifies expression of an endogenous protein, or both. In particular embodiments, a recombinant or heterologous polynucleotide encoding a desired protein is operably linked to a promoter. A recombinant or heterologous polynucleotide that modifies expression of an endogenous protein may correspond to an endogenous, heterologous or synthetic regulatory element that controls expression of the endogenous protein, or it may encode a metabolic pathway enzyme whose expression results in the attenuation of expression of the endogenous protein, or the like.

In some embodiments, the modified methanotrophic bacteria comprise a second nucleic acid molecule encoding a targeting RNA, wherein the targeting RNA comprises an sgRNA. In certain embodiments, the targeting RNA comprises a crRNA. In some embodiments, the targeting RNA comprising a crRNA further comprises a heterologous nucleic acid molecule encoding a tracrRNA. In some embodiments, a targeting RNA comprising a crRNA further comprises an endogenous tracrRNA. In some embodiments, the second nucleic acid molecule encoding the targeting RNA further encodes a self-cleaving ribozyme sequence at the 5′-end, 3′-end, or both ends of the targeting RNA. Examples of self-cleaving ribozymes include hepatitis delta virus (HDV), hammerhead, glmS, hairpin, and Varkud satellite (VS) ribozymes. The self-cleaving ribozyme sequence may be a polynucleotide sequence corresponding to SEQ ID NO:8 or SEQ ID NO:9. In some embodiments, the second nucleic acid molecule encoding the targeting RNA further comprises a transcriptional terminator. The transcriptional terminator can be a polynucleotide sequence corresponding to SEQ ID NO:7, SEQ ID NO:13, SEQ ID NO:14, SEQ ID NO:15, SEQ ID NO:16, or SEQ ID NO:17.

In some embodiments, genetically engineered or modified methanotrophic bacteria further comprise a fourth nucleic acid molecule encoding at least one recombinase. In certain embodiments, a nucleic acid molecule encoding at least one recombinase is a lambda Red recombinase. In further embodiments, a nucleic acid molecule encoding a recombinase comprises a nucleic acid molecule encoding a Bet, or functional fragments or variants thereof, operably linked a regulatory element (e.g., a promoter). In certain embodiments, a nucleic acid molecule encoding a recombinase can further comprise a nucleic acid molecule encoding an Exo, a Gam, or any combination thereof, operably linked to at least one regulatory element. In some embodiments, a nucleic acid molecule encoding a recombinase comprises a nucleic acid molecule encoding a Bet, an Exo, and a Gam, wherein the Bet, Exo, and Gam are arranged in a polycistronic operon. In certain embodiments, a nucleic acid molecule encodes a recombinase having an amino acid sequence set forth in any one of SEQ ID NOS: 10, 11, 12, or any combination thereof.

In other embodiments, a nucleic acid molecule encoding at least one recombinase is a Rac prophage recombinase. In some embodiments, a nucleic acid encoding a recombinase comprises a nucleic acid molecule encoding RecE or functional fragments or variants thereof, RecT or functional fragments or variants thereof, or both, operably linked to at least one regulatory element. In further embodiments, a nucleic acid encoding a recombinase comprises a nucleic acid molecule encoding RecE and RecT, wherein RecE and RecT are arranged in a polycistronic operon.

The genetically modified methanotrophic bacteria of the present disclosure may be cultured under a variety of culture conditions to promote the integration or expression of one or more recombinant or heterologous polynucleotides. The culture medium employed in the methods may be a liquid or solid medium. When used as a host expression system for the production of a desired product, modified methanotrophic cells are typically cultured in a liquid culture medium.

As used herein, the term “culturing” or “cultivation” refers to growing a population of microbial cells under suitable conditions in a liquid or a solid medium. In some embodiments, culturing refers to fermentative bioconversion of a C₁ substrate by methanotrophic bacteria into an intermediate or an end product.

In further embodiment, the C₁ substrate or carbon feedstock is selected methane, methanol, syngas, natural gas or combinations thereof. More typically, a carbon feedstock is selected from methane or natural gas. Methods for growth and maintenance of methanotrophic bacterial cultures are well known in the art.

In certain embodiments, a desired product is produced during a specific phase of cell growth (e.g., lag phase, log phase, stationary phase, or death phase). In some embodiments, modified methanotrophic bacteria as provided herein are cultured to a low to medium cell density (OD₆₀₀) and then production of a desired product is initiated. In some embodiments, a desired product is produced while the modified methanotrophic bacteria are no longer dividing or dividing very slowly.

In some embodiments, a desired product is produced only during stationary phase. In some embodiments, a desired product is produced during log phase and stationary phase.

When culturing is done in a liquid culture medium, the gaseous C₁ substrates may be introduced and dispersed into a liquid culture medium using any of a number of various known gas-liquid phase systems as described in more detail herein below. When culturing is done on a solid culture medium, the gaseous C₁ substrates are introduced over the surface of the solid culture medium.

Conditions sufficient to produce a desired product include culturing the modified methanotrophic bacteria at a temperature in the range of about 0° C. to about 55° C. In some embodiments, the culture temperature is in the range of about 25° C. to about 50° C. In some embodiments, the culture temperature is in the range of about 37° C. to about 50° C., and may be in the range of about 37° C. to about 45° C. Other conditions sufficient to produce a desired product include culturing the modified methanotrophs at a pH in the range of about 6 to about 9, or in the range of about 7 to about 8.

In certain embodiments, modified methanotrophic bacteria provided herein produce a desired product at about 0.001 g/L of culture to about 500 g/L of culture. In some embodiments, the amount of desired product produced is about 1 g/L of culture to about 100 g/L of culture. In some embodiments, the amount of desired product produced is about 0.001 g/L, 0.01 g/L, 0.025 g/L, 0.05 g/L, 0.1 g/L, 0.15 g/L, 0.2 g/L, 0.25 g/L, 0.3 g/L, 0.4 g/L, 0.5 g/L, 0.6 g/L, 0.7 g/L, 0.8 g/L, 0.9 g/L, 1 g/L, 2.5 g/L, 5 g/L, 7.5 g/L, 10 g/L, 12.5 g/L, 15 g/L, 20 g/L, 25 g/L, 30 g/L, 35 g/L, 40 g/L, 45 g/L, 50 g/L, 60 g/L, 70 g/L, 80 g/L, 90 g/L, 100 g/L, 125 g/L, 150 g/L, 175 g/L, 200 g/L, 225 g/L, 250 g/L, 275 g/L, 300 g/L, 325 g/L, 350 g/L, 375 g/L, 400 g/L, 425 g/L, 450 g/L, 475 g/L, or 500 g/L.

A variety of culture methodologies may be used for modified methanotrophic bacteria described herein. For example, methanotrophic bacteria may be grown by batch culture or continuous culture methodologies. In certain embodiments, the cultures are grown in a controlled culture unit, such as a fermenter, bioreactor, hollow fiber membrane bioreactor, or the like. Other suitable methods include classical batch or fed-batch culture or continuous or semi-continuous culture methodologies. In certain embodiments, the cultures are grown in a controlled culture unit, such as a fermenter, bioreactor, hollow fiber membrane bioreactor, and the like.

A classical batch culturing method is a closed system where the composition of the media is set at the beginning of the culture and not subject to external alterations during the culture process. Thus, at the beginning of the culturing process, the media is inoculated with the desired mutant methanotrophic bacteria and growth or metabolic activity is permitted to occur without adding anything further to the system. Typically, however, a “batch” culture is batch with respect to the addition of the methanotrophic substrate and attempts are often made at controlling factors such as pH and oxygen concentration. In batch systems, the metabolite and biomass compositions of the system change constantly up to the time the culture is terminated. Within batch cultures, cells moderate through a static lag phase to a high growth logarithmic phase and finally to a stationary phase where growth rate is diminished or halted. If untreated, cells in the stationary phase will eventually die. Cells in logarithmic growth phase are often responsible for the bulk production of end product or intermediate in some systems. Stationary or post-exponential phase production can be obtained in other systems.

The Fed-Batch system is a variation on the standard batch system. Fed-Batch culture processes comprise a typical batch system with the modification that the methanotrophic substrate is added in increments as the culture progresses. Fed-Batch systems are useful when catabolite repression is apt to inhibit the metabolism of the cells and where it is desirable to have limited amounts of the C₁ substrate in the media. Measurement of the actual substrate concentration in Fed-Batch systems is difficult and is therefore estimated on the basis of the changes of measureable factors, such as pH, dissolved oxygen, and the partial pressure of waste gases such as CO₂. Batch and Fed-Batch culturing methods are common and known in the art (see, e.g., Thomas D. Brock, Biotechnology: A Textbook of Industrial Microbiology, 2^{nd} Ed. (1989) Sinauer Associates, Inc., Sunderland, MA; Deshpande, Appl. Biochem. Biotechnol. 36:227, 1992, which methods are incorporated herein by reference in their entirety).

Continuous cultures are “open” systems where a defined culture media is added continuously to a bioreactor and an equal amount of conditioned media is removed simultaneously for processing. Continuous cultures generally maintain the cells at a constant high liquid phase density where cells are primarily in logarithmic phase growth. Alternatively, continuous culture may be practiced with immobilized cells where the methanotrophic substrate and nutrients are continuously added and valuable products, by-products, and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limited nutrient, such as the C1 substrate or nitrogen level, at a fixed rate and allow all other parameters to modulate. In other systems, a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art.

Liquid phase bioreactors (e.g., stirred tank, packed bed, one liquid phase, two liquid phase, hollow fiber membrane) are well known in the art and may be used for growth of modified microorganisms and biocatalysis.

By using gas phase bioreactors, substrates for bioproduction are absorbed from a gas by modified microorganisms, cell lysates or cell-free fractions thereof, rather than from a liquid. Use of gas phase bioreactors with microorganisms is known in the art (see, e.g., U.S. Pat. Nos. 2,793,096; 4,999,302; 5,585,266; 5,079,168; and 6,143,556; U.S. Statutory Invention Registration H1430; U.S. Pat. Appl. Pub. No. US 2003/0032170; Emerging Technologies in Hazardous Waste Management III, 1993, eds. Tedder and Pohland, pp. 411-428, all of which are incorporated herein by reference). Exemplary gas phase bioreactors include single pass system, closed loop pumping system, and fluidized bed reactor. By utilizing gas phase bioreactors, methane or other gaseous substrates is readily available for bioconversion by polypeptides with, for example, monooxygenase activity. In certain embodiments, methods for converting a gas into a desired product are performed in gas phase bioreactors. In further embodiments, methods for converting a gas into a desired product are performed in fluidized bed reactors. In a fluidized bed reactor, a fluid (i.e., gas or liquid) is passed upward through particle bed carriers, usually sand, granular-activated carbon, or diatomaceous earth, on which microorganisms can attach and grow. The fluid velocity is such that particle bed carriers and attached microorganisms are suspended (i.e., bed fluidization). The microorganisms attached to the particle bed carriers freely circulate in the fluid, allowing for effective mass transfer of substrates in the fluid to the microorganisms and increased microbial growth. Exemplary fluidized bed reactors include plug-flow reactors and completely mixed reactors. Uses of fluidized bed reactors with microbial biofilms are known in the art (e.g., Pfluger et al., Bioresource Technol. 102:9919, 2011; Fennell et al., Biotechnol, Bioengin. 40:1218, 1992; Ruggeri et al., Water Sci. Technol. 29:347, 1994; U.S. Pat. Nos. 4,032,407; 4,009,098; 4,009,105; and 3,846,289, all of which are incorporated herein by reference).

Methanotrophic bacteria described in the present disclosure may be grown as an isolated pure culture, with a heterologous non-methanotrophic bacteria that may aid with growth, or one or more different strains or species of methanotrophic bacteria may be combined to generate a mixed culture.

In alternative embodiments, methods described herein use modified methanotrophic bacteria of the present disclosure or cell lysates thereof immobilized on, within, or behind a solid matrix. In further embodiments, the non-naturally occurring methanotrophs of the present disclosure, cell lysates or cell-free extracts thereof are in a substantially non-aqueous state (e.g., lyophilized). Modified microorganisms, cell lysates or cell-free fractions thereof are temporarily or permanently attached on, within, or behind a solid matrix within a bioreactor. Nutrients, substrates, and other required factors are supplied to the solid matrices so that the cells may catalyze the desired reactions. Modified microorganisms may grow on the surface of a solid matrix (e.g., as a biofilm). Modified microorganisms, cell lysates or cell-free fractions derived thereof may be attached on the surface or within a solid matrix without cellular growth or in a non-living state. Exemplary solid matrix supports for microorganisms include polypropylene rings, ceramic bio-rings, ceramic saddles, fibrous supports (e.g., membrane), porous glass beads, polymer beads, charcoal, activated carbon, dried silica gel, particulate alumina, Ottawa sand, clay, polyurethane cell support sheets, and fluidized bed particle carrier (e.g., sand, granular-activated carbon, diatomaceous earth, calcium alginate gel beads).

### EXAMPLES

### Example 1

### Construction of a Crispr/Cas System for Use in Methanotrophic Bacteria

In the following example, the experiments were designed to adapt a CRISPR/Cas9 system from S. pyogenes for use in methanotrophic bacteria and test the efficiency of the system in Methylococcus capsulatus.

Escherichia coli cultures were propagated at 37° C. in Lysogeny Broth (LB). Where necessary, LB medium was solidified with 1.5% (w/v) agar and/or supplemented with 30 μg/ml kanamycin. M. capsulatus Bath cultures were grown in 25 mL MM-W1 medium in 125 mL serum bottles containing a 1:1 (v/v) methane:air gas mixture. The composition of the medium MM-W1 was as follows: 0.8 mM MgSO₄*7H₂O, 10 mM NaNO₃, 0.14 mM CaCl₂, 1.2 mM NaHCO₃, 2.35 mM KH₂PO₄, 3.4 mM K₂HPO₄, 20.7 μM Na₂MoO₄*2H₂O, 1 μM CuSO₄*5H₂O, 10 μM Fe^{III}-Na-EDTA, and 1 mL per liter of trace metals solution (containing, per liter 500 mg FeSO₄*7H₂O, 400 mg ZnSO₄*7H₂O, 20 mg MnCl₂*7H₂O, 50 mg CoCl₂*6H₂O, 10 mg NiCl₂*6H₂O, 15 mg H₃BO₃, 250 mg EDTA). Phosphate, bicarbonate, and Fe^{III}-Na-EDTA were added after the media was autoclaved and cooled. Where necessary, liquid MM-W1 media was supplemented with 15 μg/ml kanamycin (Sigma Aldrich). M. capsulatus Bath cultures were incubated with 250 rpm agitation at 42° C. When required, MM-W1 medium was solidified with 1.5% (w/v) agar and supplemented with 7.5 μg/ml kanamycin. Agar plates were incubated at 42° C. in a gas-tight chamber containing a 1:1 (v/v) methane:air gas mixture.

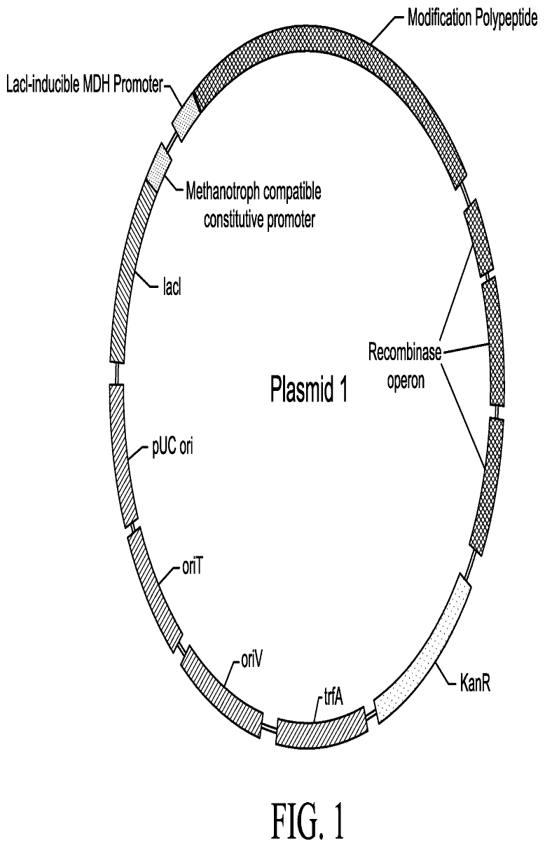

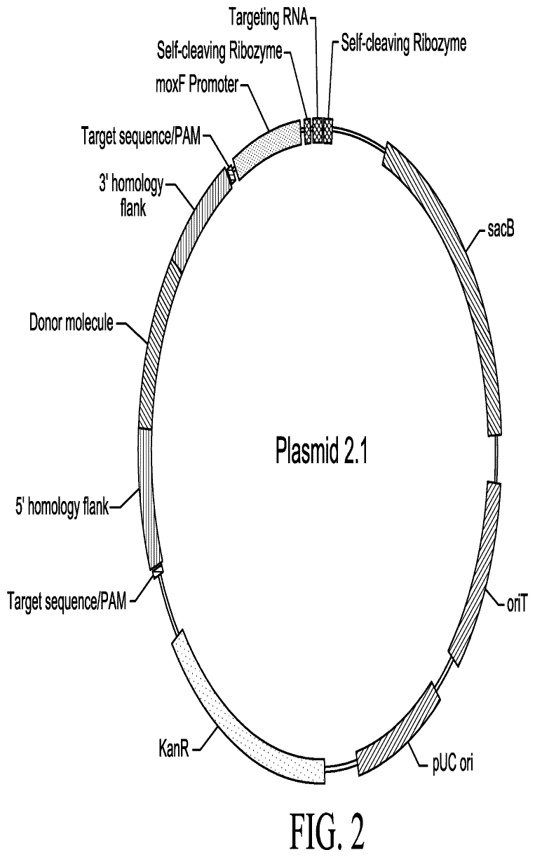

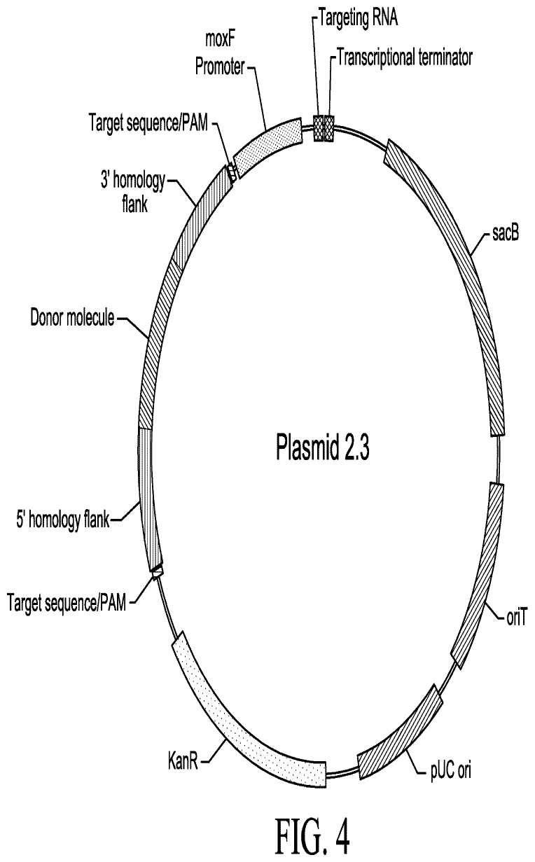

A CRISPR/Cas9 system was adapted as a genome engineering tool for Methylococcus capsulatus Bath by combining elements from CRISPR/Cas9 and lambda Red recombinase. For this purpose two plasmids were constructed. The oriV-based expression Plasmid 1 (see FIG. 1) contained a modification polypeptide (a copy of S. pyogenes wild type cas9 (codon-optimized for M. capsulatus Bath, encoding SEQ ID NO:1)) and a recombinase (a copy of the lambda Red operon (exo, SEQ ID NO:12; bet, SEQ ID NO:11; and gam, SEQ ID NO:10)), under control of an inducible methanotroph specific promoter (IPTG inducible MDH promoter, SEQ ID NO:3). Plasmid 2.1 and variants thereof (Plasmid 2.2 and Plasmid 2.3) (see, FIGS. 2-4, respectively) were pUC-based plasmids and unable to replicate in methanotrophic bacteria. Furthermore these plasmids contained an integration polynucleotide cassette comprising a donor molecule (spectinomycin resistance marker (functional in M. capsulatus Bath)) flanked by a 703 bp 5′ homology flank segment and a 657 bp 3′ homology flank segment that were homologous to the 5′ upstream and 3′ downstream sequences of the target DNA alcohol dehydrogenase (ADH, MCA0775), respectively. The 5′ homology flank segment and 3′ homology flank segment were in turn flanked by the ADH target sequence/PAM sequence. The ADH target sequence comprises a sequence that is complementary to the DNA-targeting domain of the sgRNA encoded on the same plasmid. Plasmids 2.1, 2.2, and 2.3 also harbored a targeting RNA (sgRNA) comprising a DNA targeting domain that is complementary to the ADH gene, under the control of either a methanotroph-specific (e.g., moxF (SEQ ID NO:6)) or a synthetic promoter. Plasmids 2.1, 2.2, and 2.3 differed from each other in that: Plasmid 2.3 contained the sgRNA operably linked to a methanotroph specific moxF promoter (SEQ ID NO:6) and a transcriptional terminator (SEQ ID NO:7); Plasmid 2.1 carried a self-cleaving ribozyme upstream (hammerhead ribozyme, SEQ ID NO:8) and downstream (HDV ribozyme, SEQ ID NO:9) of the sgRNA, with the sgRNA and ribozymes operably linked to the same methanotroph specific moxF promoter (SEQ ID NO:6); Plasmid 2.2 contained an sgRNA operably linked to a synthetic promoter and a transcriptional terminator (SEQ ID NO:7) (see FIGS. 2-4).

The adapted CRISPR/Cas9 system was tested with the goal of disrupting chromosomal ADH with a spectinomycin resistance marker. For this purpose Plasmid 1 was introduced into M. capsulatus Bath by conjugation yielding strain 5002365. M. capsulatus Bath wild type was grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, an Escherichia coli S17-λ pir donor strain containing Plasmid 1 was grown under standard conditions as described above and in the presence of 30 μg/ml kanamycin for 16 h. The culture was diluted to an OD₆₀₀ of 0.05 and then grown further in the presence of 30 μg/ml kanamycin until reaching an OD 600 of 0.5. Cells were harvested from 3 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 100 μL aliquots (undiluted and 1:100 dilution) were spread onto MM-W1 agar plates containing 7.5 μg/mL kanamycin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 7.5 μg/mL kanamycin to confirm kanamycin resistance as well as to further isolate transformed M. capsulatus Bath cells from residual E. coli donor cells. The presence of Plasmid 1 in M. capsulatus Bath was verified by PCR and sequencing.

Subsequently Plasmid 2.1, Plasmid 2.2, or Plasmid 2.3 were introduced into strain S002365 and wild type M. capsulatus Bath. Conjugations were performed as described above except that the mating suspension was spotted onto dry MM-W1 agar plates containing 0.2% yeast extract supplemented with 0, 1, or 5 mM IPTG. Plates were incubated for 48 h at 37° C. in the presence of a 1:1 mixture of methane and air. After 48 h, cells were re-suspended in 0.5 mL sterile MM-W1 medium. Aliquots of 100-μL and 400-μL were spread onto MM-W1 agar plates containing 2.5 μg/mL spectinomycin. The plates were incubated in gas-tight chambers containing a 1:1 mixture of methane and air and maintained at 42° C. The gas mixture was replenished every 2 days until colonies formed, typically after 5 days.

Transformation of strain S002365 with Plasmid 2.1 yielded between 5 and 33 spectinomycin resistant colonies, while transformation of S002365 with Plasmid 2.2 resulted in 496 and 736 spectinomycin resistant colonies, respectively. When S002365 was transformed with Plasmid 2.3, 11 to 33 spectinomycin resistant colonies were obtained. Generally the number of transformants increased with increasing IPTG concentration. Control strains that were transformed with Plasmid 2.1, Plasmid 2.2 or Plasmid 2.3, and did not express a copy of the Cas9 protein, yielded 10-100 times more spectinomycin resistant colonies when compared to the analogous Cas9 expressing strains. Replacement of ADH with the spectinomycin resistance cassette in the transformants was examined by PCR screen. A maximum of 66 spectinomycin resistant colonies from each transformation were screened for the presence of the spectinomycin resistance cassette in the correct location on the M. capsulatus Bath chromosome. Two sets of primers were used in this screen. Set 1 consisted of a forward primer binding upstream of the ADH 5′-homologous region on the chromosome and a reverse primer binding on the 3′-end of the spectinomycin resistance cassette. Set 2 consisted of a forward primer binding at the 5′-end of the spectinomycin resistance cassette and a reverse primer binding downstream of the MCA0775 3′-homologous region on the chromosome. Using these two primer combinations, 30% of the transformants that had received Plasmid 2.1 screened positive for the presence of the spectinomycin resistance marker in the correct location. About 90% of the transformants that had received Plasmid 2.2 screened positive for the presence of the spectinomycin resistance marker in the correct location and 20% of the transformants that had received Plasmid 2.3 screened positive for the presence of the spectinomycin resistance marker in the correct location. All of the transformants obtained from the controls were negative for the presence of the spectinomycin resistance in the correct chromosomal location. A total of 5-7 clones from each transformation that had tested positive for the presence of the spectinomycin resistance marker in the chromosome were subjected to another round of PCR. This time the primers used were binding approximately 750 bp upstream of the ADH 5′ homology flank segment and approximately 750 bp downstream of the ADH 3′ homology flank segment to confirm that the chromosomal region flanking the spectinomycin resistance cassette was still intact. All PCR products possessed the correct sequence.

In sum, these data confirm that the adapted CRISPR/Cas9 system is functional in M. capsulatus Bath. Using this system, chromosomal ADH was successfully replaced with a spectinomycin resistance marker.

### Example 2

### Generation of Genetically Engineered M. Capsulatus Using CRISPR/CAS9

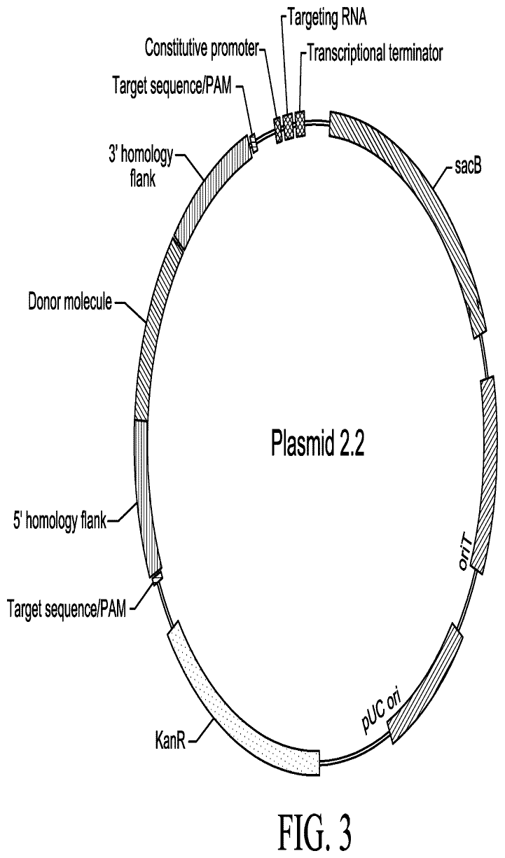

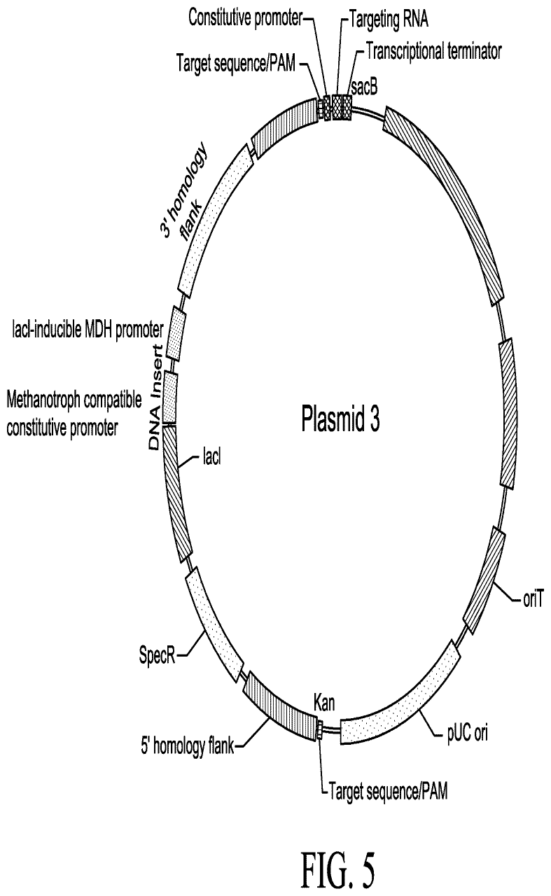

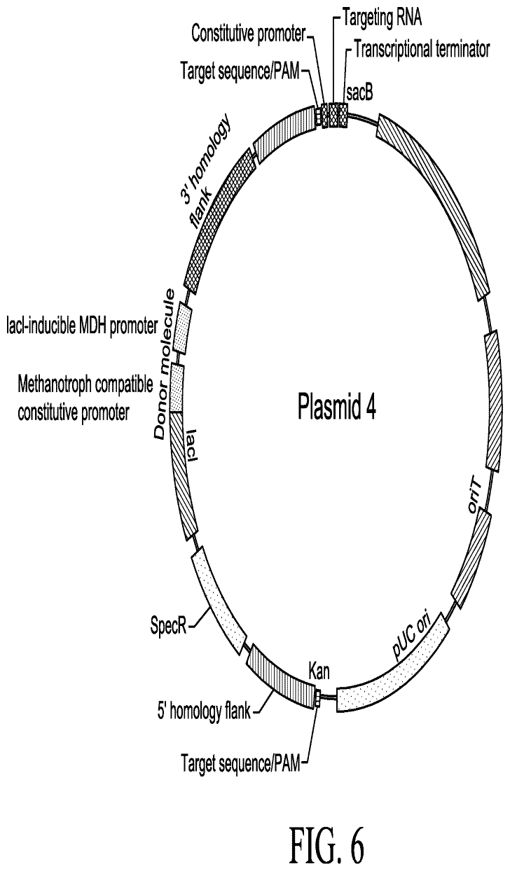

To further validate the heterologous CRISPR-Cas9 system in M. capsulatus Bath, four plasmids containing an integration polynucleotide cassette comprising different IPTG inducible metabolic pathway enzymes were constructed. For this purpose a polynucleotide integration cassette comprising a spectinomycin resistance marker, lad repressor, an IPTG inducible methanotroph-specific MDH promoter (SEQ ID NO:3), which are all flanked by a 703 bp 5′ homology flank segment and a 657 bp 3′ homology flank segment (same as described in Example 1) that were homologous to the 5′ upstream and 3′ downstream sequences of the target DNA alcohol dehydrogenase (ADH, MCA0775), respectively. The 5′ homology flank segment and 3′ homology flank segment were in turn flanked by an ADH target sequence/PAM sequence. The ADH target sequence comprises sequence that is complementary to the DNA-targeting domain of the sgRNA encoded on plasmid 3. The 5,627 bp integration polynucleotide cassette was then combined with a suicide vector backbone (derived from Plasmid 2.2) containing a Kanamycin resistance marker, a pUC origin of replication (an origin of replication for E. coli), which is non-functional in M. capsulatus Bath, an origin of transfer (oriT), a counter selection marker (sacB), and a targeting RNA (MCA0775-specific sgRNA) operably linked to a synthetic promoter and a transcriptional terminator (SEQ ID NO:7) (see Example 1, FIG. 3). The integration polynucleotide cassette was amplified and contained 20-bp overhangs complementary to the 5′ or 3′ end of the suicide vector backbone. The suicide vector backbone was amplified and contained 20-bp overhangs complementary to the 5′ or 3′ end of the integration polynucleotide cassette. Gibson cloning of the two fragments yielded Plasmid 3 (see, FIG. 5). Subsequently, four different heterologous metabolic pathway enzyme genes were cloned under control of an IPTG-inducible methanotroph-specific MDH promoter (SEQ ID NO:3) into the integration polynucleotide cassette of Plasmid 3, replacing DNA insert yielding versions of Plasmid 4 (see, FIG. 6). Plasmid 4.1 comprises a first heterologous gene encoding a first metabolic pathway enzyme as the donor molecule. Plasmid 4.2 comprises a second heterologous gene encoding a second metabolic pathway enzyme as the donor molecule. Plasmid 4.3 comprises a third heterologous gene encoding a third metabolic pathway enzyme as the donor molecule. Plasmid 4.4 comprises a fourth heterologous gene encoding a fourth metabolic pathway enzyme as the donor molecule. These plasmids were introduced into M. capsulatus Bath strain S002365, which possesses Plasmid 1 comprising a modification polypeptide (Cas9) and recombinase. Conjugations were performed as described above. Plates were incubated for 48 h at 37° C. in the presence of a 1:1 mixture of methane and air. After 48 h, cells were re-suspended in 0.5 mL sterile MM-W1 medium. 100-μL and 400-R1 aliquots were spread onto MM-W1 agar plates containing 2.5 μg/mL spectinomycin. The plates were incubated in gas-tight chambers containing a 1:1 mixture of methane and air and maintained at 42° C. The gas mixture was replenished every 2 days until colonies formed, typically after 5 days. Transformation of strain 5002365 with Plasmid 4.1, Plasmid 4.2, Plasmid 4.3 and Plasmid 4.4 yielded on average 100 spectinomycin resistant colonies. Replacement of MCA0755 with the IPTG inducible first, second, third, and fourth metabolic pathway enzymes in the obtained transformants was tested by PCR screen. A total of 64 spectinomycin resistant colonies from each transformation were screened for the presence of the integration polynucleotide cassette in the correct location on the Bath chromosome. The set of screening primers consisted of a forward primer binding upstream of the MCA0775 5′ homology flank segment on the chromosome and a reverse primer binding downstream of the MCA0775 5′ homology flank segment on the chromosome. Using this primer combination, 13% of the transformants that had received Plasmid 4.1 screened positive for the presence of the IPTG inducible first heterologous metabolic pathway enzyme in the correct chromosomal location. About 27% of the transformants that had received Plasmid 4.2 screened positive for the presence of the IPTG inducible second heterologous metabolic pathway enzyme in the correct location. 53% of the transformants that had received Plasmid 4.3 screened positive for the presence of the IPTG inducible third heterologous metabolic pathway enzyme in the correct location and 11% of the transformants that had received Plasmid 4.4 screened positive for the presence of the IPTG inducible fourth heterologous metabolic pathway enzyme in the correct location. All of the PCR products possessed the correct sequence, confirming replacement of MCA0775 with the IPTG inducible first, second, third, or fourth heterologous metabolic pathway enzymes.

Overall, using this system the chromosomal MDH (MCA0775) was successfully replaced with four different IPTG inducible heterologous metabolic pathway enzymes. These data confirm that DNA fragments up to 5 kb can be integrated into the Bath genome using a heterologous Bath CRISPR/Cas9 system.

### Example 3

### Demonstration of Cas9 Kill Constructs for M. Capsulatus

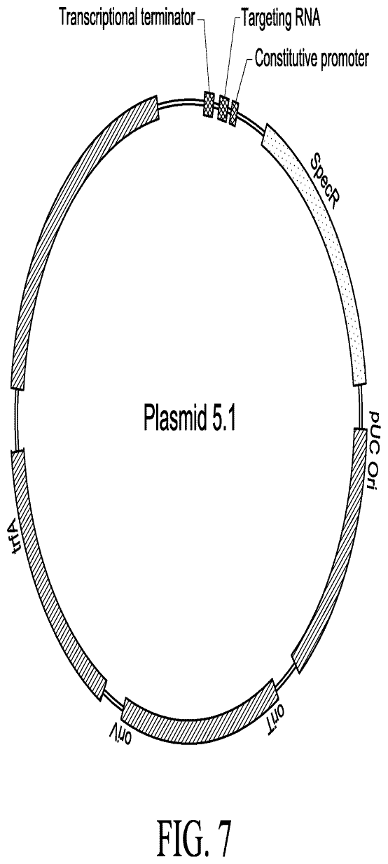

To validate the function of a heterologous CRISPR-Cas9 system in M. capsulatus Bath, three plasmids (5.1, 5.2, and 5.3) containing a constitutively transcribed guide RNA targeting alcohol dehydrogenase gene (MCA0775) were constructed. An oriV-based “Kill” plasmid contained a targeting RNA (sgRNA) comprising a DNA targeting domain that is complementary to the ADH gene, under the control of a synthetic promoter and followed by a transcriptional terminator (FIG. 7). Plasmids 5.1, 5.2 and 5.3 differed from each other in that each contained a targeting RNA having a different 20-23 bp DNA targeting domain that is complementary to a target sequence in the ADH gene. Plasmids 5.2 and 5.3 were constructed by amplifying plasmid 5.1 with phosphorylated primers containing sequence complementary to the 23 bp DNA targeting domain of the sgRNA sequence as an extension to the 5′ primer and ligating the PCR product.

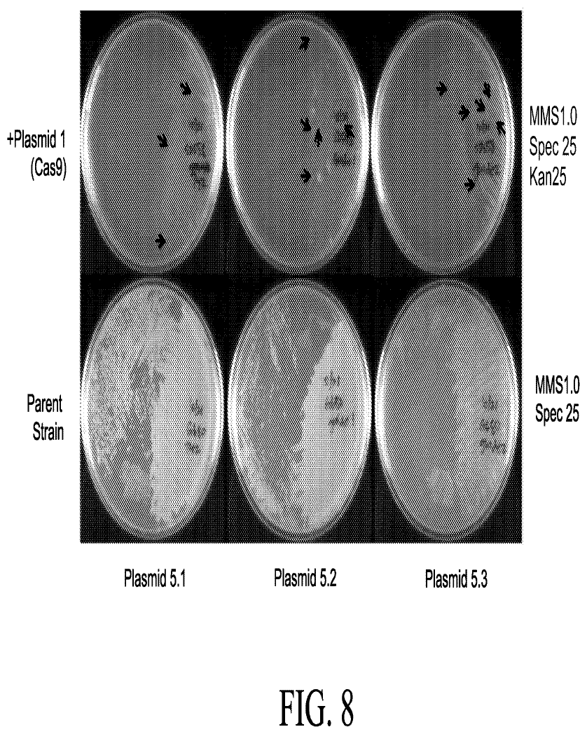

The “Kill” plasmid was tested with the goal of cleaving the M. capsulatus genome in the absence of a DNA template to modify the target site. For this purpose, Plasmids 5.1, 5.2, or 5.3 were introduced into M. capsulatus Bath strain 5002365 cells, which comprised a modification polypeptide (Cas9) and recombinase (see, Example 1), or wild type M. capsulatus Bath. M. capsulatus Bath wild type and 5002365 were grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, Escherichia coli DH10B donor strains containing Plasmid 5.1, 5.2 or 5.3 and pRK2013 helper strain were grown under standard conditions as described above and in the presence of 50 μg/ml spectinomycin or 50 μg/ml kanamycin, respectively, for 16 h. The culture was diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 100 μL aliquots (undiluted and 1:100 dilution) were spread onto MM-W1 agar plates containing 7.5 μg/mL spectinomycin. The plates were incubated in gas-tight chambers containing a 1:1 mixture of methane and air and maintained at 42° C. The gas mixture was replenished every 2 days until colonies formed, typically after 5-7 days. As shown in FIG. 8, the wild-type M. capsulatus Bath cells comprising the MCA0775-targeted cleavage Plasmid 5.1, 5.2, or 5.3 only without Cas9 activity (bottom row) shows a large number of colonies for each MCA0775-targeting RNA, since oriV backbone allows for propagation in M. capsulatus Bath host and absence of cas9 allows for no cleavage of genomic DNA at MCA0775. However, 5002365 cells comprising both the Cas9 containing Plasmid 1 and MCA0775-targeted disruption Plasmid 5.1, 5.2, or 5.3 shows few colonies because the presence of each MCA0775-targeting RNA and cas9 results in the formation of a DNA cleaving enzyme which cleaves the host's genomic DNA at MCA0775. In sum, these data confirm that CRISPR/cas9 is active in M. capsulatus Bath. Using an sgRNA and cas9 in the absence of any additional DNA for homologous recombination resulted in DNA cleavage and thus cell death.

### Example 4

### Generation of MCA1474 and MCA0229 Deletions in M. Capsulatus Using CRISPR/CAS9

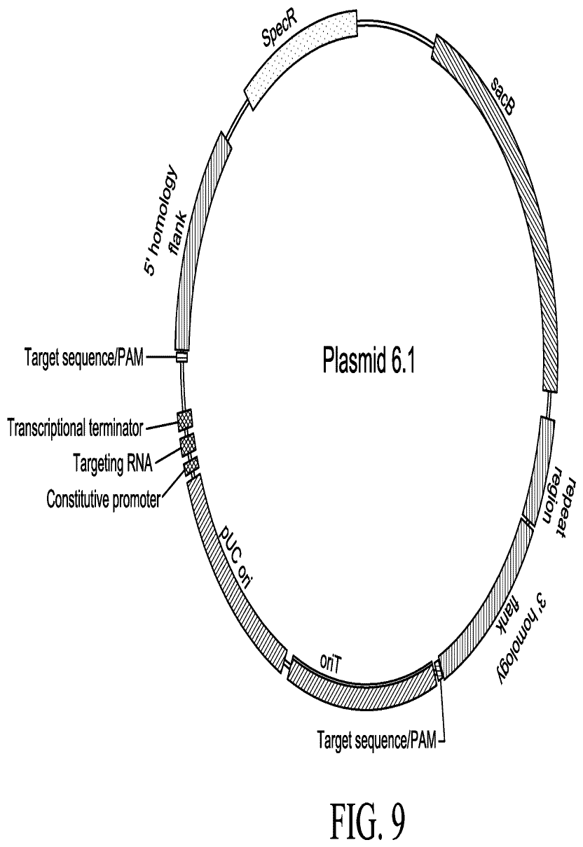

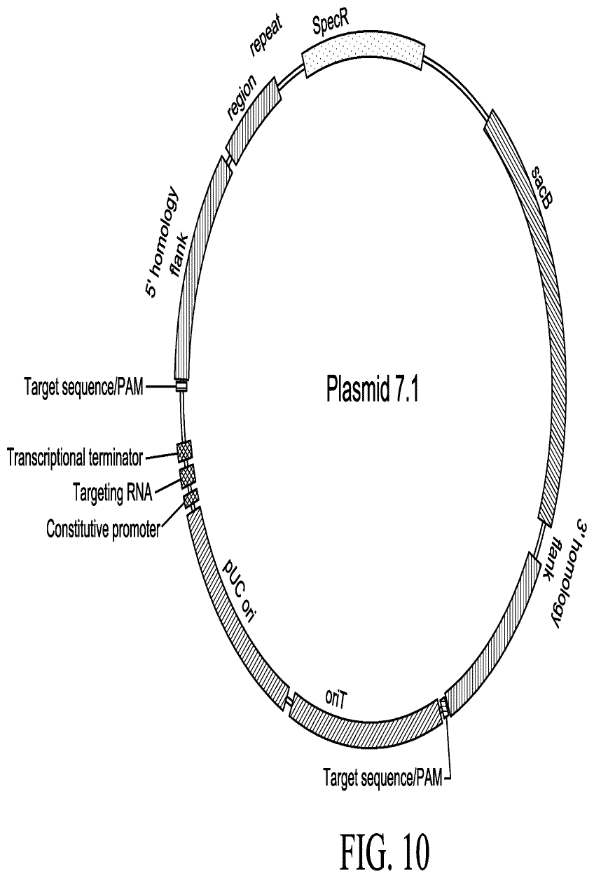

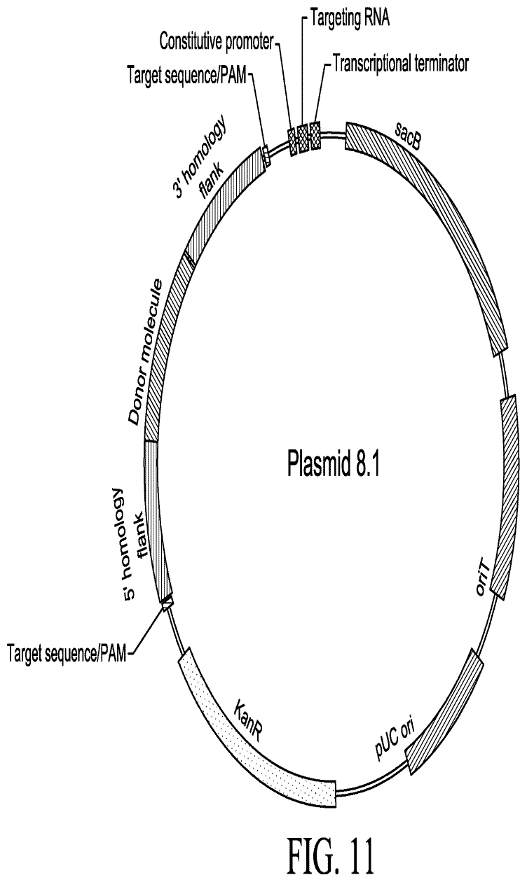

To further validate the heterologous CRISPR-Cas9 system in M. capsulatus Bath, two plasmids, plasmid 6.1 and plasmid 7.1, containing a deletion polynucleotide cassette comprising selectable markers were constructed to target genes of interest MCA1474 or MCA0229, respectively. For this purpose a polynucleotide deletion cassette comprising donor molecule, a spectinomycin resistance marker and the Bacillus subtilis sacB gene which confers sucrose sensitivity, flanked by a 752-853 bp 5′ homology flank and a 536-838 bp 3′ homology flank that were homologous to the 5′ upstream and 3′ downstream sequences of the target gene of interest, respectively. These plasmids also harbored a targeting RNA (sgRNA) comprising a DNA targeting domain that is complementary to the gene of interest, under control of a synthetic terminator and a transcriptional terminator. In plasmid 6.1, a loopout region (repeat region) which was homologous to a 500 bp region upstream of the 5′ homology flank on the methanotroph genome target site was positioned downstream of the sacB gene on the plasmid, and conversely in plasmid 7.1, a loopout region (repeat region) which was homologous to a 311 bp region downstream of the 3′ homology flank on the methanotroph genome target site was positioned upstream of the spectinomycin resistance gene on the plasmid (FIGS. 9-10). This loopout region (repeat region) would allow for subsequent marker removal by selecting against sacB gene on sucrose containing media. A control plasmid, plasmid 8.1, was constructed containing a spectinomycin resistance marker flanked by a 703 bp 5′ homology flank and a 657 bp 3′ homology flank that were homologous to the 5′ upstream and 3′ downstream sequences of the target DNA alcohol dehydrogenase (ADH, MCA0775), respectively. Plasmids 2.1, 2.2, and 2.3 also harbored a targeting RNA (sgRNA) comprising a DNA targeting domain that is complementary to the ADH gene, operably linked to a synthetic promoter and a transcriptional terminator (SEQ ID NO:7) (FIG. 11).

The CRISPR/Cas9 system was tested with the goal of disrupting MCA1474 or MCA0229 in a markerless fashion. For this purpose, Plasmids 6.1 or 7.1 were introduced into M. capsulatus Bath strain S002365 cells, which comprised a modification polypeptide (Cas9) and recombinase (see, Example 1), and wild-type M. capsulatus Bath. S002365 and wild-type M. capsulatus Bath were grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, Escherichia coli DH10B donor strains containing Plasmid 6.1, 7.1 or 8.1 and pRK2013 helper strain were grown under standard conditions as described above and in the presence of 50 μg/ml spectinomycin or 50 μg/ml kanamycin, respectively, for 16 h. The culture was diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 100 μL aliquots (undiluted and 1:100 dilution) were spread onto MM-W1 agar plates containing 7.5 μg/mL spectinomycin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 7.5 μg/mL spectinomycin to confirm spectinomycin resistance as well as to further isolate transformed M. capsulatus Bath cells from residual E. coli donor cells.

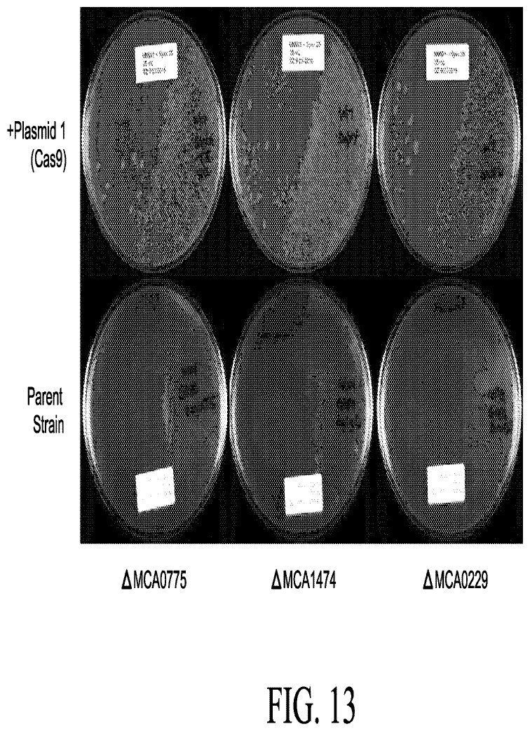

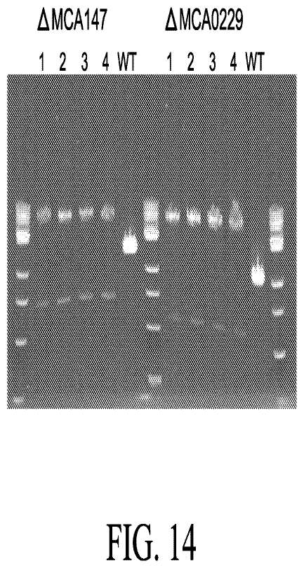

Transformation of strain 5002365 with plasmid 6.1, 7.1 and 8.1 yielded a few hundreds of spectinomycin resistant colonies, while transformation of wild-type M. capsulatus Bath yielded no colonies (FIG. 13). Deletion of gene of interest with deletion cassette in the transformants was examined by PCR screen. A maximum of 8 spectinomycin resistant colonies from each transformation were screened for the presence of the spectinomycin resistance cassette in the correct location on the M. capsulatus Bath chromosome. Three sets of primers were used in this screen. Set 1 consisted of a forward primer binding upstream of MCA1474 5′-homologous region on the chromosome and a reverse primer binding downstream of MCA1474 3′-homologous region on the chromosome. Set 2 consisted of a forward primer binding upstream of MCA0229 5′-homologous region on the chromosome and a reverse primer binding downstream of MCA0229 3′-homologous region on the chromosome. Set 3 consisted of a forward primer binding upstream of the ADH 5′-homologous region on the chromosome and a reverse primer binding on the 3′-end of the spectinomycin resistance cassette. PCR products of set 1 and 2 were sent for sequencing for presence of the spectinomycin resistance marker. Using these primers to screen conjugations of 5002365 with plasmid 6.1, 7.1 and 8.1 respectively, about 90% of transformants that had received plasmid 6.1 screened positive for the presence of the spectinomycin resistance marker in the correct location. About 90% of the transformants that had received Plasmid 7.1 screened positive for the presence of the spectinomycin resistance marker in the correct location. About 90% of the transformants that had received Plasmid 8.1 screened positive for the presence of the spectinomycin resistance marker in the correct location (FIGS. 13-14).

Overall, these data confirm that using this system, genes of interest were targeted for deletion at a high frequency with the heterologous CRISPR/Cas9 system.

### Example 5

### Generation of Single Stranded DNA Mediated Mutants with Lambda Red Operon

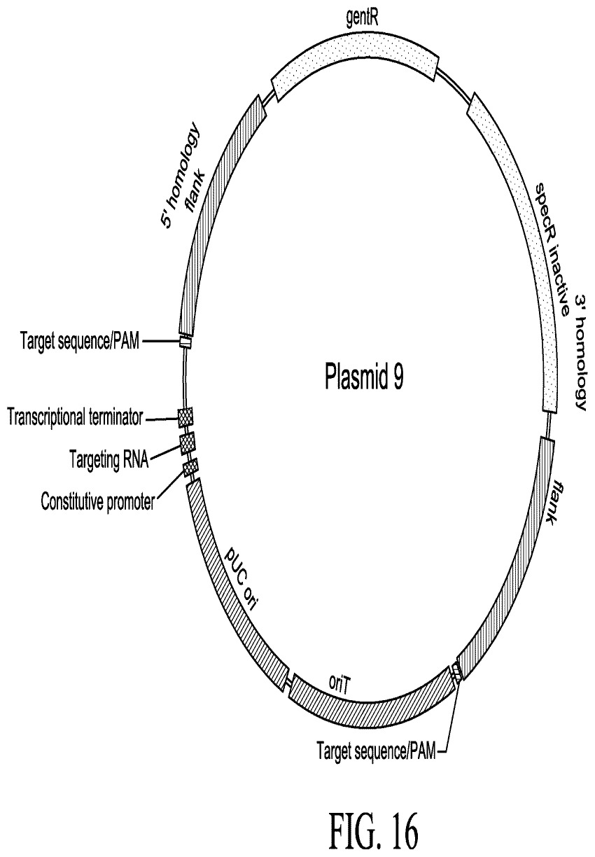

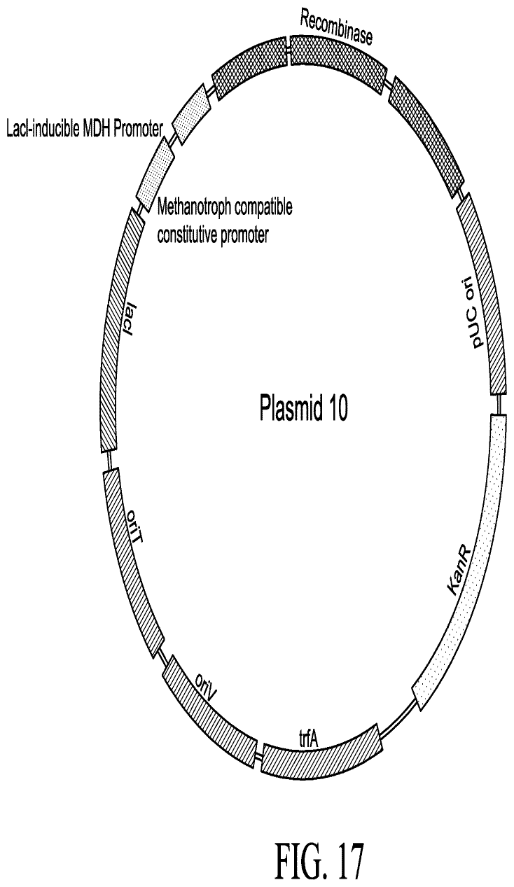

To demonstrate the use of lambda red for enhancing homologous recombination, two plasmids were constructed and a single-stranded oligonucleotide was synthesized. Plasmid 9 (FIG. 16) is a pUC-based plasmid and unable to replicate in methanotrophic bacteria. Furthermore, this plasmid contains an integration polynucleotide cassette comprising an inactivated spectinomycin resistance marker (G274T, T275A) and gentamicin resistance marker (functional in M. capsulatus Bath) flanked by a 703 bp 5′ homology flank segment and a 657 bp 3′ homology flank segment that were homologous to the 5′ upstream and 3′ downstream sequences of the target DNA alcohol dehydrogenase (ADH, MCA0775), respectively. Plasmid 9 also harbored a targeting RNA (sgRNA) comprising a DNA targeting domain that is complementary to the ADH gene, under the control of a synthetic promoter and a transcriptional terminator (SEQ ID NO:7). An oriV-based expression Plasmid 10 (FIG. 17) contained a recombinase (a copy of the lambda Red operon: exo, SEQ ID NO:12; bet, SEQ ID NO:11; and gam, SEQ ID NO:10), under control of an inducible methanotroph specific promoter (IPTG inducible MDH promoter, SEQ ID NO:3). A 75-bp oligonucleotide comprised 2 bp mutation (G274, T275) to restore function to the spectinomycin resistance marker, flanked by a 38 bp 5′ homology flank segment and a 35 bp 3′ homology flank segment that were homologous to the 5′ upstream and 3′ downstream sequences of the target 2 bp mutation of the spectinomycin resistance marker (SEQ ID NO:18).

The use of lambda red for enhancing homologous recombination was tested with the goal restoring function to the inactivated spectinomycin resistance marker with an oligonucleotide containing a 2 bp mutation which restored function. For this purpose, Plasmid 9 was first introduced into M. capsulatus Bath by conjugation yielding strain S009934. M. capsulatus Bath S002365 was grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, Escherichia coli DH10B donor strains containing Plasmid 9 and Escherichia coli pRK2013 helper strain were grown under standard conditions as described above and in the presence of 30 μg/ml gentamicin or 50 μg/ml kanamycin, respectively, for 16 h. The culture was diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 100 μL aliquots (undiluted and 1:100 dilution) were spread onto MM-W1 agar plates containing 15 μg/mL gentamicin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 15 μg/mL gentamicin to confirm gentamicin resistance as well as to further isolate transformed M. capsulatus Bath cells from residual E. coli donor cells. Replacement of ADH with the functional gentamicin resistance marker and the inactive spectinomycin resistance marker in the transformants was verified by PCR and sequencing.

Plasmid 10 was introduced into strain 5009934 to yield S010104. Conjugations were performed as described above except aliquots of 100-μL and 400-μL were spread onto MM-W1 agar plates containing 25 μg/mL kanamycin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 25 μg/mL kanamycin to confirm kanamycin resistance as well as to further isolate transformed M. capsulatus Bath cells from residual E. coli donor cells. The presence of Plasmid 10 in M. capsulatus Bath was verified by PCR and sequencing.

Subsequently, the 75-bp oligonucleotide (SEQ ID NO:18) was electroporated into M. capsulatus strain S009934 and S010104 cells. M. capsulatus Bath strain 5009934 and 5010104 were grown under standard conditions (as described above), and 5010104 strain culture was supplemented with 15 μg/ml kanamycin, for 16 h or until the culture reached an optical density at 600 nm (OD₆₀₀) of 1. Half of S010104 cells were additionally grown with 1 mM IPTG for 2 h. Cells were harvested from 5 ml of each culture, washed three times with sterile water and then resuspended in 50 μl of sterile water. Cells were mixed with 10 or none, of the 75-bp oligonucleotide and transferred into a cuvette (0.1 cm gap, BioRad). Cells were pulsed at 1.8 mV, 200Ω, 25 g. After pulse, cells were immediately transferred to 2 ml of MM-W1.0 and recovered under standard conditions (as described above). After 4 h, cells were pelleted and entire pellet was spread onto MM-W1 agar plates containing 15 μg/mL spectinomycin. The plates were incubated in gas-tight chambers containing a 1:1 mixture of methane and air and maintained at 42° C. The gas mixture was replenished every 2 days until colonies formed, typically after 5-7 days.

Transformation of S009934 with no 75-bp oligonucleotide (SEQ ID NO:18) yielded 453 spectinomycin resistant colonies, while transformation of S009934 with 10 μg 75-bp oligonucleotide yielded 375 spectinomycin resistant colonies. When S0010104 was transformed with 10 μg 75-bp oligonucleotide, 267 spectinomycin resistant colonies were obtained, while S0010104 grown with IPTG then subsequently transformed with 10 μg 75-bp oligonucleotide yielded 1,021 spectinomycin resistant colonies. A maximum of 40 spectinomycin resistant colonies from each transformation was screened by PCR and sequencing for the presence of the 2 bp mutation introduced by the oligonucleotide in the spectinomycin resistance marker integrated in the chromosome. Based on the sequencing data, 0% of the S009934 transformed with no oligonucleotide screened positive for the 2 bp mutation, whereas about 25% of the S009934 transformed with 10 μg 75-bp oligonucleotide (SEQ ID NO:18) screened positive for the 2 bp mutation. Additionally, when S010104 was transformed with 10 ug 75-bp oligonucleotide, about 50% of colonies screened positive for the 2 bp mutation and when S010104 was grown with IPTG first and then transformed with 10 ug 75-bp oligonucleotide (SEQ ID NO:18), 100% of colonies screened positive for the 2 bp mutation.

In summary, these data confirm that the lambda red operon is functional in M. capsulatus Bath. This system was successful in increasing the mutation frequency of an oligonucleotide, which restored function to the inactivated spectinomycin resistance marker, and in combination with application of the cas9 method, resulted in 100% efficiency in introducing the targeted mutation.

### Example 6

### Generation of RS15395 Deletion in M. Capsulatus Bath Using the Lambda Red Operon

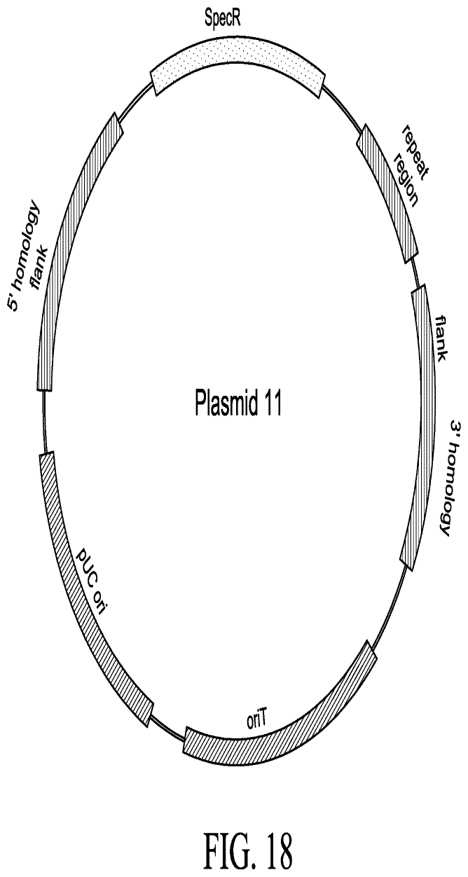

To further validate the heterologous lambda red operon in M. capsulatus bath, a pUC-based plasmid, Plasmid 11 (FIG. 18), containing a deletion polynucleotide cassette comprising a selectable marker was constructed to target gene of interest RS15395. For this purpose, a polynucleotide deletion cassette comprising a donor molecule, a spectinomycin resistance marker, flanked by a 760 bp 5′ homology flank segment and a 374-767 bp 3′ homology flank segment that were homologous to the 5′ upstream and 3′ downstream sequences of the target gene of interest, respectively. This 2,832 bp fragment was amplified by PCR to generate double stranded linear DNA (dsDNA) for electroporation.

Plasmid 10 was introduced into M. capsulatus wild type strain to yield LR1 strain. M. capsulatus Bath wild type was grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, Escherichia coli DH10B donor strains containing Plasmid 10 and Escherichia coli pRK2013 helper strain were grown under standard conditions as described above and in the presence of 50 μg/ml kanamycin for 16 hr. The cultures were diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 1000_, aliquots (undiluted and 1:100 dilution) were spread onto MM-W1 agar plates containing 25 μg/mL kanamycin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 25 μg/mL kanamycin to confirm gentamicin resistance as well as to further isolate transformed M. capsulatus Bath cells from residual E. coli donor cells. The presence of Plasmid 10 in M. capsulatus Bath was verified by PCR and sequencing.

Subsequently, the 2,832 bp dsDNA was electroporated into M. capsulatus wild type, LR1 cells. M. capsulatus Bath wild type, LR1 strain were grown under standard conditions (as described above), and LR1 strain culture was supplemented with 15 μg/ml kanamycin, for 16 h or until the culture reached an optical density at 600 nm (OD₆₀₀) of 1. LR1 cells were additionally grown with 1 mM IPTG for 2 h. Cells were harvested from 5 ml of each culture, washed three times with sterile water and then resuspended in 50 IA of sterile water. Cells were mixed with 5 μg, 1 μg, or none, of the 2,832-bp oligonucleotide and transferred into a cuvette (0.1 cm gap, BioRad). Cells were pulsed at 1.8 mV, 200Ω, 25 μF. After pulse, cells were immediately transferred to 2 ml of MM-W1.0 and recovered under standard conditions (as described above). After 4 h, cells were pelleted and entire pellet was spread onto MM-W1 agar plates containing 15 μg/mL spectinomycin. The plates were incubated in gas-tight chambers containing a 1:1 mixture of methane and air and maintained at 42° C. The gas mixture was replenished every 2 days until colonies formed, typically after 5-7 days.

Transformation of wild type with no 2,832 bp dsDNA yielded 46 spectinomycin resistant colonies, while transformation of LR1 yielded 6 spectinomycin resistant colonies. Transformation of wild type with 1 μg 2,832 bp dsDNA yielded 30 spectinomycin resistant colonies, while transformation of LR1 yielded 60 colonies. Transformation of wild type with 5 μg 2,832 bp dsDNA yielded 34 colonies, while transformation of LR1 yielded 747 colonies. A maximum of 8 spectinomycin resistant colonies from each transformation was screened by PCR and sequencing for the spectinomycin resistance marker at the targeted site in the chromosome. Based on the sequencing data, 0% of the wild type transformed with the 2,832 bp dsDNA contained the spectinomycin resistance marker at the targeted site whereas 75% of LR1 transformed with 1 μg 2,832 bp dsDNA contained the insertion of spectinomycin resistance marker at the targeted site and 100% of LR transformed with 5 μg 2,832 bp dsDNA contained the insertion of spectinomycin resistance marker at the targeted site in the chromosome.

In sum, using this system the chromosomal gene RS15395 was successfully replaced with the spectinomycin resistance marker using a double stranded linear DNA. These data confirm that linear DNA fragments up to 2.8 kb can be integrated into the M. capsulatus Bath genome via heterologous expression of the lambda red operon.

### Example 7

### Demonstration of CAS9 System with or without Lambda Red

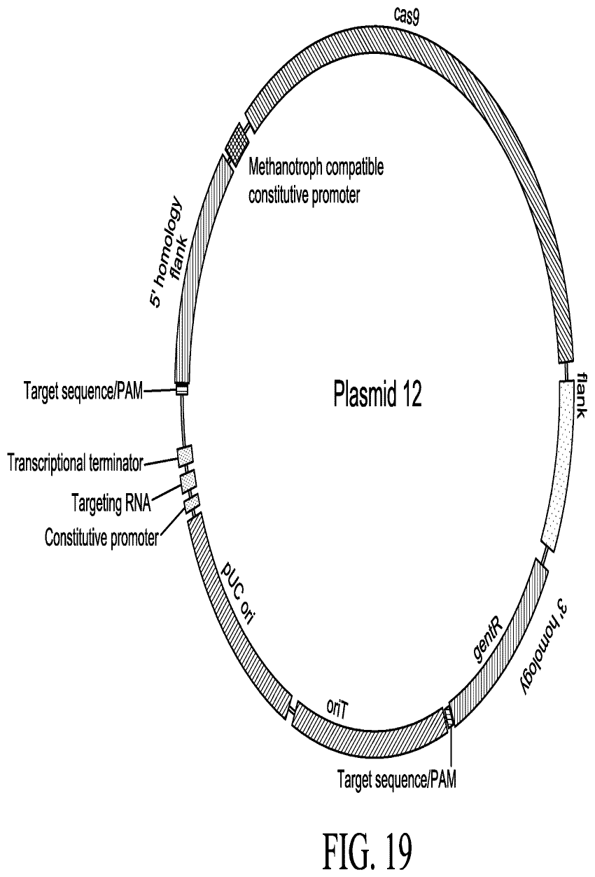

To further validate the heterologous CRISPR-Cas9 system in M. capsulatus Bath, the CRISPR/Cas9 system was used to test the necessity of lambda red element for genetic engineering. For this purpose a plasmid, Plasmid 12 (FIG. 19), containing an integration polynucleotide cassette comprising a gentamicin resistance marker, cas9 operably linked to a methanotroph compatible constitutive promoter, which were all flanked by a 753 bp 5′ homology flank segment and a 783 bp 3′ homology flank segment that were homologous to the 5′ upstream and 3′ downstream sequences of the target gene glucose-1-phosphate adenylyl transferase (glgC, MCA1474), respectively. The 6,718 bp integration polynucleotide cassette was then combined with a suicide vector backbone containing a pUC origin of replication, which is non-functional in M. capsulatus Bath, an origin of transfer (oriT) and a targeting RNA (MCA1474-specific sgRNA) operably linked to a synthetic promoter and a transcriptional terminator. This plasmid was introduced into M. capsulatus Bath strain 5002365, which possesses Plasmid 1 comprising Cas9 and recombinase, by conjugation to yield 5010475. M. capsulatus Bath 5002365 was grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, Escherichia coli DH10B donor strains containing Plasmid 12 and Escherichia coli pRK2013 helper strain were grown under standard conditions as described above and in the presence of 30 μg/ml gentamicin or 50 μg/ml kanamycin, respectively, for 16 h. The culture was diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 100 μL aliquots (undiluted and 1:100 dilution) were spread onto MM-W1 agar plates containing 15 μg/mL gentamicin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 15 μg/mL gentamicin to confirm gentamicin resistance as well as to further isolate transformed M. capsulatus Bath cells from residual E. coli donor cells. Replacement of glgC with the functional gentamicin resistance marker and cas9 gene in the transformants was verified by PCR and sequencing.

Next, Plasmid 10 was introduced into M. capsulatus S010475 to yield S010477, S010478, and 510479. M. capsulatus Bath wild type was grown under standard conditions (as described above) for 24 h or until the culture reached an optical density at 600 nm (OD₆₀₀) of 1. Cells were harvested from 1.5 ml of this culture, washed three times with MM-W1 medium and then re-suspended in 0.5 ml MM-W1. In parallel, Escherichia coli DH10B donor strains containing Plasmid 10 and Escherichia coli pRK2013 helper strain were grown under standard conditions as described above and in the presence of 50 μg/ml kanamycin for 16 h. The cultures were diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and 100 μL aliquots (undiluted and 1:100 dilution) were spread onto MM-W1 agar plates containing 25 μg/mL kanamycin. The plates were incubated in gas-tight chambers containing a 1:1 mixture of methane and air and maintained at 42° C. The gas mixture was replenished every 2 days until colonies formed, typically after 5-7 days. Colonies were streaked onto MM-W1 agar plates containing 25 μg/mL kanamycin to confirm gentamicin resistance as well as to further isolate transformed M. capsulatus Bath cells from residual E. coli donor cells. The presence of Plasmid 10 in M. capsulatus Bath was verified by PCR and sequencing.

Finally, to test the integration efficiency of Cas9 with and without lambda red operon, Plasmid 7.1 was introduced into M. capsulatus Bath strain S010475, or S010477, S010478, and S10479 cells, which comprised a modification polypeptide (Cas9) without or with recombinase, respectively. S010475, or S010477, S010478, and S10479 cells were grown under standard conditions (as described above) for 24 h or until the culture reached an optical density at 600 nm (OD₆₀₀) of 1, and the latter three strains were supplemented with 15 μg/mL kanamycin. Cells were harvested from 1.5 ml of this culture, washed three times with MM-W1 medium and then re-suspended in 0.5 ml MM-W1. In parallel, Escherichia coli DH10B donor strains containing Plasmid 7.1 and pRK2013 helper strain were grown under standard conditions as described above and in the presence of 50 μg/ml spectinomycin or 50 μg/ml kanamycin, respectively, for 16 h. The culture was diluted to an OD₆₀₀ of 1.5. Cells were harvested from 1 ml of the culture, washed three times with MM-W1 medium and then combined with 0.5 ml of the M. capsulatus Bath suspension. The mixed suspension was pelleted, re-suspended in 40 μL of MM-W1 medium and spotted onto dry MM-W1 agar plates containing 0.2% yeast extract. Plates were incubated for 48 hrs. at 37° C. in the presence of a 1:1 mixture of methane and air. After 48 h, cells were re-suspended in 1 mL sterile MM-W1 medium and were spread onto MM-W1 agar plates containing 15 μg/mL spectinomycin. The plates were incubated in gas-tight chambers containing a 1:1 mixture of methane and air and maintained at 42° C. The gas mixture was replenished every 2 days until colonies formed, typically after 5-7 days.

Transformation of S010475, which contains only cas9, with Plasmid 7.1 yielded no spectinomycin resistant colonies, while transformation of Plasmid 7.1 into S010477, S010478, and S010479, which contain cas9 and lambda red recombinase, yielded 3-13 colonies. In summary, cas9 alone was not sufficient to result in genomic integration, whereas cas9 in conjunction with lambda red yielded genomic integration.

While specific embodiments of the invention have been illustrated and described, it will be readily appreciated that the various embodiments described above can be combined to provide further embodiments, and that various changes can be made therein without departing from the spirit and scope of the invention.

All of the U.S. patents, U.S. patent application publications, U.S. patent applications, foreign patents, foreign patent applications and non-patent publications referred to in this specification, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

That which is claimed is:

1. A method of altering the genome of a methanotrophic bacterium, comprising culturing under conditions and for a time sufficient to allow expression in a methanotrophic bacterium of a site-specific polynucleotide modification system;
wherein the methanotrophic bacterium contains a heterologous nucleic acid molecule encoding the site-specific polynucleotide modification system that is operably linked to a regulatory element in a vector, the nucleic acid molecule comprising:
(a) a first heterologous nucleic acid molecule encoding a modification polypeptide, wherein the modification polypeptide comprises a targeting RNA binding domain and a site-specific nuclease domain, and
(b) a second heterologous nucleic acid molecule encoding a targeting RNA, wherein the targeting RNA comprises a duplex-forming region and a DNA-targeting domain,
wherein the complex of the expressed modification polypeptide with the expressed targeting RNA binds to and cleaves a genomic target sequence of the methanotrophic bacterium,
thereby site-specifically altering the genome of the methanotrophic bacterium.

2. The method of claim 1, wherein the first heterologous nucleic acid molecule encoding the modification polypeptide encodes a Cas9 polypeptide.

3. The method of claim 1 or 2, wherein the nucleic acid molecule encoding the Cas9 polypeptide is codon optimized for methanotrophic bacteria.

4. The method of claim 3, wherein the nucleic acid molecule encoding the Cas9 polypeptide is codon optimized for Methylococcus capsulatus Bath or Methylosinus trichosporium OB3b.

5. The method of any one of claims 1-4, wherein the first heterologous nucleic acid molecule comprises a polynucleotide sequence encoding a polypeptide having at least 80% sequence identity to SEQ ID NO:1.

6. The method of any one of claims 1-4, wherein the encoded Cas9 polypeptide is at least 80% identical to a polypeptide corresponding to Cas9 of Streptococcus pyogenes.

7. The method of any one of claims 1-6, wherein the targeting RNA comprises a crRNA comprising a DNA-targeting domain and a duplex-forming region.

8. The method of claim 7, further comprising introducing into the methanotrophic bacterium a nucleic acid molecule encoding a tracrRNA comprising a duplex-forming region complementary to the duplex-forming region of the crRNA of claim 7.

9. The method of any one of claims 1-6, wherein the targeting RNA comprises an sgRNA.

10. The method of any one of the preceding claims, wherein the second heterologous nucleic acid molecule encoding the targeting RNA further encodes a self-cleaving ribozyme located at the 5′-end, 3′-end, or both ends of the targeting RNA.

11. The method of claim 10, wherein the self-cleaving ribozyme comprises a polynucleotide sequence corresponding to SEQ ID NO:8 or SEQ ID NO:9.

12. The method of any one of the preceding claims, wherein the second heterologous nucleic acid molecule encoding the targeting RNA further comprises a transcriptional terminator.

13. The method of claim 12, wherein the transcriptional terminator comprises a polynucleotide sequence corresponding to any one of SEQ ID NOS:7 and 13-17.

14. The method of any one of the preceding claims, wherein the cleaved genomic target sequence is repaired by non-homologous end joining, by homology-directed repair, or a combination thereof.

15. The method of any one of the preceding claims, wherein the methanotrophic bacteria further contain a third heterologous nucleic acid molecule comprising an integration polynucleotide.

16. The method of claim 15, wherein the integration polynucleotide comprises a 5′-homology flank comprised of at least about 20 nucleotides and a 3′-homology flank comprised of at least about 20 nucleotides.

17. The method of claim 15 or 16, wherein the integration polynucleotide genetically modifies a gene of the methanotrophic bacterium.

18. The method of claim 15 or 16, wherein the integration polynucleotide genetically modifies a regulatory element of the methanotrophic bacterium.

19. The method of claim 17 or 18, wherein the integration polynucleotide introduces a point mutation, frameshift mutation, deletion, substitution, insertion, or any combination thereof.

20. The method of any one of claims 15-19, wherein the integration polynucleotide comprises a donor molecule.

21. The method of claim 20, wherein the donor molecule comprises a nucleic acid molecule encoding a heterologous protein.

22. The method of claim 20, wherein the encoded a heterologous protein is a reporter protein.

23. The method of claim 20, wherein the encoded a heterologous protein is
(a) an amino acid biosynthesis enzyme;
(b) an isoprene synthase, crotonase, crotonyl CoA thioesterase, 4-oxalocrotonate decarboxylase, or any combination thereof;
(c) a fatty acid converting enzyme;
(d) a fatty acid elongation pathway enzyme;
(e) a carbohydrate biosynthesis enzyme; or
(f) a lactate dehydrogenase.

24. The method of claim 20, wherein the donor molecule comprises a nucleic acid molecule encoding a homologous or endogenous methanotrophic bacterial protein.

25. The method of any one of claims 15-22, wherein the integration polynucleotide comprises a selectable marker.

26. The method of claim 25, wherein the selectable marker is an antibiotic resistance protein.

27. The method of any one of claims 15-26, wherein the integration polynucleotide is contained in a vector.

28. The method of claim 27, wherein the integration polynucleotide further comprises a 5′-target sequence, a 3′-target sequence, and a PAM sequence, wherein the 5′-target and 3′-target sequences are targeted by the targeting RNA and the PAM sequence is targeted by the modification polypeptide.

29. The method of claim 27 or claim 28, wherein the vector comprises a counter-selectable marker.

30. The method of claim 27, wherein the vector comprises a temperature sensitive origin of replication or an origin of replication that is non-functional in the methanotrophic bacterium.

31. The method of any one of the preceding claims, wherein the methanotrophic bacterium further contains a fourth heterologous nucleic acid molecule encoding a recombinase.

32. The method of claim 31, wherein the recombinase comprises lambda recombinase Exo, Bet, Gam, or any combination thereof, RecA recombinase, or Rac recombinase RecE, RecT, or both RecE and RecT.

33. The method of any one of the preceding claims, wherein the first heterologous nucleic acid molecule and the second heterologous nucleic acid molecule are contained in the same vector.

34. The method of any one of the preceding claims, wherein the first heterologous nucleic acid molecule and the second heterologous nucleic acid molecule are contained in different vectors.

35. The method of claim 31 or 32, wherein the first heterologous nucleic acid molecule, the second heterologous nucleic acid molecule, and the fourth heterologous nucleic acid molecule are contained in the same vector.

36. The method of claim 31 or 32, wherein the first heterologous nucleic acid molecule encoding the modification polypeptide and the second heterologous nucleic acid molecule encoding the targeting RNA are contained in different vectors and at least one vector further comprises the fourth heterologous nucleic acid molecule.

37. The method of claim 31, 32, or 35 wherein the first heterologous nucleic acid molecule and the fourth heterologous nucleic acid molecule are arranged in a polycistronic operon.

38. The method of any one of claim 31, 32, 35 or 37, wherein the first heterologous nucleic acid molecule and the fourth heterologous nucleic acid molecule are operably linked to the same regulatory element.

39. The method of any one of claims 1-38, wherein the first heterologous nucleic acid molecule and the second heterologous nucleic acid molecule are operably linked to the same regulatory element.

40. The method of any one of claims 1-38, wherein the first heterologous nucleic acid molecule and the second heterologous nucleic acid molecule are operably linked to different regulatory elements.

41. The method of any one of the preceding claims, wherein any one of the regulatory elements comprise a host promoter, an exogenous promoter, or a non-natural promoter.

42. The method of any one of the preceding claims, wherein at least one regulatory element comprises an inducible promoter.

43. The method of any one of the preceding claims, wherein the regulatory element of the first heterologous nucleic acid molecule comprises an inducible promoter.

44. The method of any one of the preceding claims, wherein the regulatory element of the second heterologous nucleic acid molecule is a promoter comprising a polynucleotide sequence corresponding to SEQ ID NO:5 or SEQ ID NO:6.

45. The method of any one of the preceding claims, wherein the methanotrophic bacterium is selected from a Methylococcus, Methylomonas, Methylomicrobium, Methylobacter, Methylocaldum, Methylovulum, Methylomarinum, Methylocystis, and Methylosinus.

46. The method of any one of the preceding claims, wherein the methanotrophic bacterium is a Methylococcus capsulatus Bath, Methylosinus trichosporium OB3b, Methylomonas 16a, Methylosinus sporium, Methylocystis parvus, Methylomonas methanica, Methylomonas albus, Methylobacter capsulatus, Methylobacterium organophilum, Methylomonas sp AJ-3670, Methylocella silvestris, Methylocella palustris, Methylocella tundrae, Methylocystis daltona SB2, Methylocystis bryophila, Methylocapsa aurea KYG, Methylacidiphilum infernorum, Methylibium petroleiphilum, Methylomicrobium alcaliphilum, or combinations thereof.

47. The method of any one of the preceding claims, wherein the methanotrophic bacterium is Methylococcus capsulatus Bath or Methylosinus trichosporium OB3b.

48. A modified methanotroph, comprising a heterologous nucleic acid molecule encoding a site-specific polynucleotide modification system that is operably linked to a regulatory element in a vector, the nucleic acid molecule comprising:
(a) a first heterologous nucleic acid molecule encoding a modification polypeptide, wherein the modification polypeptide comprises a targeting RNA binding domain and a site-specific nuclease domain,
(b) a second heterologous nucleic acid molecule encoding a targeting RNA, wherein the targeting RNA comprises a duplex-forming region and a DNA-targeting domain, and
(c) a third heterologous nucleic acid molecule comprising an integration polynucleotide, wherein the expressed modification polypeptide can associate with the expressed targeting RNA to form a complex capable of binding to and cleaving a genomic target sequence of the methanotroph.

49. The modified methanotroph of claim 48, wherein the first heterologous nucleic acid molecule encoding the modification polypeptide encodes a Cas9 polypeptide.

50. The modified methanotroph of claim 48 or 49, wherein the nucleic acid molecule encoding the Cas9 polypeptide is codon optimized for methanotrophic bacteria.

51. The modified methanotroph of claim 50, wherein the nucleic acid molecule encoding the Cas9 polypeptide is codon optimized for Methylococcus capsulatus Bath or Methylosinus trichosporium OB3b.

52. The modified methanotroph of any one of claims 48-51, wherein the first heterologous nucleic acid molecule comprises a polynucleotide sequence encoding a polypeptide having at least 80% sequence identity to SEQ ID NO:1.

53. The modified methanotroph of any one of claims 48-51, wherein the encoded Cas9 polypeptide is at least 80% identical to a polypeptide corresponding to Cas9 of Streptococcus pyogenes.

54. The modified methanotroph of any one of claims 48-53, wherein the targeting RNA comprises a crRNA comprising a DNA-targeting domain and a duplex-forming region.

55. The modified methanotroph of claim 54, further comprising introducing into the methanotrophic bacteria a nucleic acid molecule encoding a tracrRNA comprising a duplex-forming region complementary to the duplex-forming region of the crRNA of claim 7.

56. The modified methanotroph of any one of claims 48-53, wherein the targeting RNA comprises an sgRNA.

57. The modified methanotroph of any one of claims 48-56, wherein the second heterologous nucleic acid molecule encoding the targeting RNA further encodes a self-cleaving ribozyme located at the 5′-end, 3′-end, or both ends of the targeting RNA.

58. The modified methanotroph of claim 57, wherein the self-cleaving ribozyme comprises a polynucleotide sequence corresponding to SEQ ID NO:8 or SEQ ID NO:9.

59. The modified methanotroph of any one of claims 48-58, wherein the second heterologous nucleic acid molecule encoding the targeting RNA further comprises a transcriptional terminator.

60. The modified methanotroph of claim 59, wherein the transcriptional terminator comprises a polynucleotide sequence corresponding to any one of SEQ ID NOS:7 and 13-17.

61. The modified methanotroph of any one of claims 48-60, wherein the cleaved genomic target sequence is repaired by non-homologous end joining, by homology-directed repair, or a combination thereof.

62. The modified methanotroph of any one of claims 48-61, wherein the integration polynucleotide comprises a 5′-homology flank comprised of at least about 20 nucleotides and a 3′-homology flank comprised of at least about 20 nucleotides.

63. The modified methanotroph of any one of claims 48-62, wherein the integration polynucleotide genetically modifies a gene of the methanotroph.

64. The modified methanotroph of any one of claims 48-63, wherein the integration polynucleotide genetically modifies a regulatory element of the methanotroph.

65. The modified methanotroph of claim 63 or 64, wherein the integration polynucleotide introduces a point mutation, frameshift mutation, deletion, substitution, insertion, or any combination thereof.

66. The modified methanotroph of any one of claims 48-65, wherein the integration polynucleotide comprises donor molecule.

67. The modified methanotroph of claim 66, wherein the donor molecule comprises a nucleic acid molecule encoding a heterologous protein.

68. The modified methanotroph of claim 66, wherein the encoded heterologous protein is a reporter protein.

69. The modified methanotroph of claim 66, wherein the encoded heterologous protein is
(a) an amino acid biosynthesis enzyme;
(b) an isoprene synthase, crotonase, crotonyl CoA thioesterase, 4-oxalocrotonate decarboxylase, or any combination thereof;
(c) a fatty acid converting enzyme;
(d) a fatty acid elongation pathway enzyme;
(e) a carbohydrate biosynthesis enzyme; or
(f) a lactate dehydrogenase.

70. The modified methanotroph of claim 66, wherein the donor molecule comprises a nucleic acid molecule encoding a homologous or endogenous methanotrophic bacterial protein.

71. The modified methanotroph of any one of claims 48-68, wherein the integration polynucleotide comprises a selectable marker.

72. The modified methanotroph of claim 71, wherein the selectable marker is an antibiotic resistance protein.

73. The modified methanotroph of any one of claims 48-72, wherein the integration polynucleotide is contained in a vector.

74. The modified methanotroph of claim 73, wherein the integration polynucleotide further comprises a 5′-target sequence, a 3′-target sequence, and a PAM sequence, wherein the 5′-target and 3′-target sequences are targeted by the targeting RNA and the PAM sequence is targeted by the modification polypeptide.

75. The modified methanotroph of claim 73 or claim 74, wherein the vector comprises a counter-selectable marker.

76. The modified methanotroph of claim 73, wherein the vector comprises a temperature sensitive origin of replication or an origin of replication that is non-functional in methanotrophic bacteria.

77. The modified methanotroph of any one of claims 48-76, wherein the methanotroph further contains a fourth heterologous nucleic acid molecule encoding a recombinase.

78. The modified methanotroph of claim 77, wherein the recombinase comprises lambda recombinase Exo, Bet, Gam, or any combination thereof, or Rac recombinase RecE, RecT, or both.

79. The modified methanotroph of any one of claims 48-78, wherein the first heterologous nucleic acid molecule and the second heterologous nucleic acid molecule are contained in the same vector.

80. The modified methanotroph of any one of claims 48-78, wherein the first heterologous nucleic acid molecule and the second heterologous nucleic acid molecule are contained in different vectors.

81. The modified methanotroph of claim 77 or 78, wherein the first heterologous nucleic acid molecule, the second heterologous nucleic acid molecule, and the fourth heterologous nucleic acid molecule are contained in the same vector.

82. The modified methanotroph of claim 77 or 78, wherein the first heterologous nucleic acid molecule encoding the modification polypeptide and the second heterologous nucleic acid molecule encoding the targeting RNA are contained in different vectors and at least one vector further comprises the fourth heterologous nucleic acid molecule.

83. The modified methanotroph of claim 77, 78, or 81 wherein the first heterologous nucleic acid molecule and the fourth heterologous nucleic acid molecule are arranged in a polycistronic operon in the same vector.

84. The modified methanotroph of any one of claim 77, 78, 81 or 83, wherein the first heterologous nucleic acid molecule and the fourth heterologous nucleic acid molecule are operably linked to the same regulatory element.

85. The modified methanotroph of any one of claims 48-84, wherein the first heterologous nucleic acid molecule and the second heterologous nucleic acid molecule are operably linked to the same regulatory element.

86. The modified methanotroph of any one of claims 48-84, wherein the first heterologous nucleic acid molecule and the second heterologous nucleic acid molecule are operably linked to different regulatory elements.

87. The modified methanotroph of any one of claims 48-86, wherein any one of the regulatory elements comprises a host promoter, an exogenous promoter, or a non-natural promoter.

88. The modified methanotroph of any one of claims 48-87, wherein at least one regulatory element comprises an inducible promoter.

89. The modified methanotroph of any one of claims 48-88, wherein the regulatory element of the first nucleic acid molecule comprises an inducible promoter.

90. The modified methanotroph of any one of claims 48-89, wherein the regulatory element of the second nucleic acid molecule is a promoter comprising a polynucleotide sequence corresponding to SEQ ID NO:5 or SEQ ID NO:6.

91. The modified methanotroph of any one of claims 48-90, wherein the methanotroph is selected from a Methylococcus, Methylomonas, Methylomicrobium, Methylobacter, Methylocaldum, Methylovulum, Methylomarinum, Methylocystis, and Methylosinus.

92. The modified methanotroph of any one of claims 48-91, wherein the methanotroph is a Methylococcus capsulatus Bath, Methylosinus trichosporium OB3b, Methylomonas 16a, Methylosinus sporium, Methylocystis parvus, Methylomonas methanica, Methylomonas albus, Methylobacter capsulatus, Methylobacterium organophilum, Methylomonas sp AJ-3670, Methylocella silvestris, Methylocella palustris, Methylocella tundrae, Methylocystis daltona SB2, Methylocystis bryophila, Methylocapsa aurea KYG, Methylacidiphilum infernorum, Methylibium petroleiphilum, Methylomicrobium alcaliphilum, or combinations thereof.

93. The modified methanotroph of any one of claims 48-92, wherein the methanotroph is Methylococcus capsulatus Bath or Methylosinus trichosporium OB3b.
